(19) United States
(12) Patent Application Publication
(10) Pub. No.: US 2022/0297171 A1
(43) Pub. Date: Sep. 22, 2022

(54) Processes For Solubilizing Municipal Solid Waste With Enzyme Compositions Comprising Protease And Enzyme Compositions Thereof

(71) Applicant: Novozymes A/S, Bagsvaerd (DK)

(72) Inventors: Hongzhi Huang, Beijing (CN); Yun Wang, Beijing (CN); Wanghui Xu, Beijing (CN)

(73) Assignee: Novozymes A/S, Bagsvaerd (DK)

(21) Appl. No.: 16/500,792

(22) PCT Filed: Apr. 14, 2018
(86) PCT No.: PCT/CN2018/083129

§ 371 Date: Oct. 3, 2019

(30) Foreign Application Priority Data
Apr. 14, 2017 (CN) PCT/CN2017/080569

(51) Int. Cl. B09B 3/60; C12N 9/54; C12N 9/58; C12P 5/02; C12N 9/42

(52) U.S. Cl. CPC B09B 3/60; C12N 9/54; C12N 9/58; C12P 5/023; C12N 9/2437; C12P 2203/00

(57) ABSTRACT

A processes for solubilization or hydrolysis of a municipal solid waste with an Enzyme composition, comprising: (i) a cellulolytic enzyme composition, and (ii) a protease selected from the group consisting of: (a) a protease having at least 50%, 55%, 60%, 65%, 70%, 75%, 80%, 85%, 90%, 95% or 100% identity to amino acids 1 to 177 of SEQ ID NO: 1 or a fragment thereof having protease activity; (b) a protease having at least 50%, 55%, 60%, 65%, 70%, 75%, 80%, 85%, 90%, 95% or 100% identity to SEQ ID NO: 5; (c) a protease having at least 50%, 55%, 60%, 65%, 70%, 75%, 80%, 85%, 90%, 95% or 100% identity to SEQ ID NO: 32 or a variant thereof; (d) a protease having at least 50%, 55%, 60%, 65%, 70%, 75%, 80%, 85%, 90%, 95% or 100% identity to SEQ ID NO: 33; and (e) a protease having at least 50%, 55%, 60%, 65%, 70%, 75%, 80%, 85%, 90%, 95% or 100% identity to amino acids 199 to 564 of SEQ ID NO: 36.

### REFERENCE TO A SEQUENCE LISTING

This application contains a Sequence Listing in computer readable form. The computer readable form is incorporated herein by reference.

### FIELD OF THE INVENTION

The present invention relates to processes for solubilization or hydrolysis of municipal solid waste with an enzyme composition comprising protease.

### BACKGROUND OF THE INVENTION

Municipal solid waste (MSW) is commonly known as trash, garbage, refuse or rubbish. It consists of solid waste fractions that typically comes from municipalities and includes, for instance, waste from homes, schools, offices, hospitals, institutions etc. MSW is produced world-wide in very large quantities. In EU alone 2.44 million tons were generated in 2012. The challenges of MSW production are many and may include collection, sorting, treatment, and disposal. Furthermore, well known environmental issues such as air and groundwater pollution from landfills is related to MSW. With an increasing world population entailing an increasing waste production, proper sustainable MSW management is a global challenge.

Enzymatic treatment of MSW provides an innovative approach in MSW management (Jensen et al., 2010, Waste Management 30, p. 2497-2503; Jensen et al., 2011, Biochem. Biotechnol. 165, p. 1799-1811; Tonini and Astrup, 2012, Waste Management 32, p. 165-176). This technology is based on a liquefaction/solubilization step of organic degradable parts with hydrolytic enzymes and subsequent separation of the MSW into a bioliquid and solids. The bioliquid can be used for biogas production while the solids can be further sorted and used for recycling or combusted according to the composition of the material. The technology has proven very robust at even high dry matter concentrations (35%) and has been demonstrated in pilot/demonstration facilities treating up to 1 ton of MSW/hour. The use of cellulases for liquefaction of MSW with subsequent separation of unsorted waste into a bioliquid—used for biogas production—and into inorganic valuable products suitable for recycling has been clearly illustrated (WO 2013/185777A1).

WO 2016/030480 and WO 2016/030472 disclose blends of different enzymes mixed with cellulase for solubilizing a model-substrate of MSW. The blends provided a synergistic effect in solubilizing the MSW compared to the individual contribution of each component thereof.

There is a need in the art for more effective enzyme compositions that can solubilize municipal solid waste.

The present invention provides such enzymes compositions and their use in processes for solubilizing municipal solid waste.

### SUMMARY OF THE INVENTION

The present invention relates to processes for solubilizing a municipal solid waste, comprising treating the municipal solid waste with an enzyme composition comprising (i) a cellulolytic enzyme composition and (ii) a protease selected from the group consisting of: (a) a protease having at least 50%, 55%, 60%, 65%, 70%, 75%, 80%, 85%, 90%, 95% or 100% identity to amino acids 1 to 177 of SEQ ID NO: 1 or a fragment thereof having protease activity; (b) a protease having at least 50%, 55%, 60%, 65%, 70%, 75%, 80%, 85%, 90%, 95% or 100% identity to SEQ ID NO: 5; (c) a protease having at least 50%, 55%, 60%, 65%, 70%, 75%, 80%, 85%, 90%, 95% or 100% identity to SEQ ID NO: 32 or a variant thereof; (d) a protease having at least 50%, 55%, 60%, 65%, 70%, 75%, 80%, 85%, 90%, 95% or 100% identity to SEQ ID NO: 33; and (e) a protease having at least 50%, 55%, 60%, 65%, 70%, 75%, 80%, 85%, 90%, 95% or 100% identity to amino acids 199 to 564 of SEQ ID NO: 36, in an effective amount to produce a solubilized municipal solid waste. In one aspect, the processes further comprise recovering the solubilized municipal solid waste.

The present invention also relates to processes for producing a fermentation product, comprising: (a) solubilizing a municipal solid waste with an effective amount of enzyme composition comprising (i) a cellulolytic enzyme composition and (ii) a protease selected from the group consisting of: (a) a protease having at least 50%, 55%, 60%, 65%, 70%, 75%, 80%, 85%, 90%, 95% or 100% identity to amino acids 1 to 177 of SEQ ID NO: 1 or a fragment thereof having protease activity; (b) a protease having at least 50%, 55%, 60%, 65%, 70%, 75%, 80%, 85%, 90%, 95% or 100% identity to SEQ ID NO: 5; (c) a protease having at least 50%, 55%, 60%, 65%, 70%, 75%, 80%, 85%, 90%, 95% or 100% identity to SEQ ID NO: 32 or a variant thereof; (d) a protease having at least 50%, 55%, 60%, 65%, 70%, 75%, 80%, 85%, 90%, 95% or 100% identity to SEQ ID NO: 33; and (e) a protease having at least 50%, 55%, 60%, 65%, 70%, 75%, 80%, 85%, 90%, 95% or 100% identity to amino acids 199 to 564 of SEQ ID NO: 36; (b) fermenting the solubilized municipal solid waste with one or more (e.g., several) fermenting microorganisms to produce the fermentation product; and (c) recovering the fermentation product from the fermentation.

The present invention also relates to processes of fermenting a municipal solid waste, comprising: fermenting the municipal solid waste with one or more (e.g., several) fermenting microorganisms, wherein the municipal solid waste is solubilized with an enzyme composition comprising (i) a cellulolytic enzyme composition and (ii) a protease selected from the group consisting of: (a) a protease having at least 50%, 55%, 60%, 65%, 70%, 75%, 80%, 85%, 90%, 95% or 100% identity to amino acids 1 to 177 of SEQ ID NO: 1 or a fragment thereof having protease activity; (b) a protease having at least 50%, 55%, 60%, 65%, 70%, 75%, 80%, 85%, 90%, 95% or 100% identity to amino acids of SEQ ID NO: 5; (c) a protease having at least 50%, 55%, 60%, 65%, 70%, 75%, 80%, 85%, 90%, 95% or 100% identity to SEQ ID NO: 32 or a variant thereof; (d) a protease having at least 50%, 55%, 60%, 65%, 70%, 75%, 80%, 85%, 90%, 95% or 100% identity to SEQ ID NO: 33; and (e) a protease having at least 50%, 55%, 60%, 65%, 70%, 75%, 80%, 85%, 90%, 95% or 100% identity to amino acids 199 to 564 of SEQ ID NO: 36. In one aspect, the fermenting of the municipal solid waste produces a fermentation product. In another aspect, the processes further comprise recovering the fermentation product from the fermentation.

The present invention also relates to enzyme compositions comprising (i) a cellulolytic enzyme composition and (ii) a protease selected from the group consisting of: (a) a protease having at least 50%, 55%, 60%, 65%, 70%, 75%, 80%, 85%, 90%, 95% or 100% identity to amino acids 1 to 177 of SEQ ID NO: 1 or a fragment thereof having protease activity; (b) a protease having at least 50%, 55%, 60%, 65%, 70%, 75%, 80%, 85%, 90%, 95% or 100% identity to amino acids of SEQ ID NO: 5; (c) a protease having at least 50%, 55%, 60%, 65%, 70%, 75%, 80%, 85%, 90%, 95% or 100% identity to SEQ ID NO: 32 or a variant thereof; (d) a protease having at least 50%, 55%, 60%, 65%, 70%, 75%, 80%, 85%, 90%, 95% or 100% identity to SEQ ID NO: 33; and (e) a protease having at least 50%, 55%, 60%, 65%, 70%, 75%, 80%, 85%, 90%, 95% or 100% identity to amino acids 199 to 564 of SEQ ID NO: 36. In one embodiment, the enzyme compositions further or even further comprise a xylanase, a beta-xylosidase, or a xylanase and a beta-xylosidase. In another embodiment, the enzyme compositions further or even further comprise one or more enzymes selected from a lipase, a mannanase, a pectinase, and a beta-glucanase.

### BRIEF DESCRIPTION OF DRAWINGS

FIGS. 1A and 1B show the effect of various proteases on the solubilization of a model municipal solid waste (MSW) substrate with cellulolytic enzyme composition CEC/M at 50° C., pH 5 for 24 hours. Cellulolytic enzyme composition CEC/M without protease was run as a control. FIG. 1A shows first-round screening of the proteases added to cellulolytic enzyme composition CEC/M. Four top candidates were selected for second-round screening under the same conditions and the results are shown in FIG. 1B.

FIG. 2 shows the dose response of several proteases on the solubilization of a model MSW substrate by cellulolytic enzyme composition CEC/M. The X-axis shows the ratio of protease based on enzyme protein. The Y-axis shows the solubilization level (percentage).

FIG. 3 shows a comparison between several lipases on the solubilization of a model MSW substrate by cellulolytic enzyme composition CEC/M. Each lipase was loaded at 0.1% (low dose) and 1% (high dose) product/TS to cellulolytic enzyme composition CEC/M, respectively.

FIG. 4 shows dose optimization of an Aspergillus aculeatus pectinase on the solubilization of a model MSW substrate by cellulolytic enzyme composition CEC/M. The X-axis shows the ratio of protease based on enzyme protein. The Y-axis shows the solubilization level (percentage).

FIG. 5 shows optimization of the ratio between cellulolytic enzyme composition CEC/M and selected enzyme candidates in a multicomponent enzyme blend on the solubilization of a model MSW substrate. The cellulolytic enzyme composition was added in an amount of 2.5% product/TS and was replaced by the component enzymes as shown in the X-axis based on enzyme protein.

FIG. 6 shows the dose/response curves for cellulolytic enzyme composition CEC/M and candidate enzyme composition Sample 2 on the solubilization of a model MSW substrate. The X-axis shows the applied enzyme concentration. The Y-axis shows the solubilization level (percentage).

FIG. 7 shows the dose/response curves for cellulolytic enzyme composition CEC, Sample 1 and Sample 2. The X-axis shows the applied enzyme concentration. The Y-axis shows the solubilization level (percentage).

### DEFINITIONS

Acetylxylan esterase: The term “acetylxylan esterase” means a carboxylesterase (EC 3.1.1.72) that catalyzes the hydrolysis of acetyl groups from polymeric xylan, acetylated xylose, acetylated glucose, alpha-napthyl acetate, and p-nitrophenyl acetate. Acetylxylan esterase activity can be determined using 0.5 mM p-nitrophenylacetate as substrate in 50 mM sodium acetate pH 5.0 containing 0.01% TWEEN™ 20 (polyoxyethylene sorbitan monolaurate). One unit of acetylxylan esterase is defined as the amount of enzyme capable of releasing 1 μmole of p-nitrophenolate anion per minute at pH 5, 25° C.

Alpha-L-arabinofuranosidase: The term “alpha-L-arabinofuranosidase” means an alpha-L-arabinofuranoside arabinofuranohydrolase (EC 3.2.1.55) that catalyzes the hydrolysis of terminal non-reducing alpha-L-arabinofuranoside residues in alpha-L-arabinosides. The enzyme acts on alpha-L-arabinofuranosides, alpha-L-arabinans containing (1,3)- and/or (1,5)-linkages, arabinoxylans, and arabinogalactans. Alpha-L-arabinofuranosidase is also known as arabinosidase, alpha-arabinosidase, alpha-L-arabinosidase, alpha-arabinofuranosidase, polysaccharide alpha-L-arabinofuranosidase, alpha-L-arabinofuranoside hydrolase, L-arabinosidase, or alpha-L-arabinanase. Alpha-L-arabinofuranosidase activity can be determined using 5 mg of medium viscosity wheat arabinoxylan (Megazyme International Ireland, Ltd., Bray, Co. Wicklow, Ireland) per ml of 100 mM sodium acetate pH 5 in a total volume of 200 μl for 30 minutes at 40° C. followed by arabinose analysis by AMINEX® HPX-87H column chromatography (Bio-Rad Laboratories, Inc.).

Alpha-glucuronidase: The term “alpha-glucuronidase” means an alpha-D-glucosiduronate glucuronohydrolase (EC 3.2.1.139) that catalyzes the hydrolysis of an alpha-D-glucuronoside to D-glucuronate and an alcohol. Alpha-glucuronidase activity can be determined according to de Vries, 1998, J. Bacteriol. 180: 243-249. One unit of alpha-glucuronidase equals the amount of enzyme capable of releasing 1 μmole of glucuronic or 4-O-methylglucuronic acid per minute at pH 5, 40° C.

Auxiliary Activity 9 polypeptide: The term “Auxiliary Activity 9 polypeptide” or “AA9 polypeptide” means a polypeptide classified as a lytic polysaccharide monooxygenase (Quinlan et al., 2011, Proc. Natl. Acad. Sci. USA 208: 15079-15084; Phillips et al., 2011, ACS Chem. Biol. 6: 1399-1406; Lin et al., 2012, Structure 20: 1051-1061). AA9 polypeptides were formerly classified into the glycoside hydrolase Family 61 (GH61) according to Henrissat, 1991, Biochem. J. 280: 309-316, and Henrissat and Bairoch, 1996, Biochem. J. 316: 695-696.

AA9 polypeptides enhance the hydrolysis of a cellulosic material by an enzyme having cellulolytic activity. Cellulolytic enhancing activity can be determined by measuring the increase in reducing sugars or the increase of the total of cellobiose and glucose from the hydrolysis of a cellulosic material by cellulolytic enzyme under the following conditions: 1-50 mg of total protein/g of cellulose in pretreated corn stover (PCS), wherein total protein is comprised of 50-99.5% w/w cellulolytic enzyme protein and 0.5-50% w/w protein of an AA9 polypeptide for 1-7 days at a suitable temperature, such as 40° C.-80° C., e.g., 40° C., 45° C., 50° C., 55° C., 60° C., 65° C., 70° C., 75° C., or 80° C. and a suitable pH, such as 4-9, e.g., 4.5, 5.0, 5.5, 6.0, 6.5, 7.0, 7.5, 8.0, 8.5, or 9.0, compared to a control hydrolysis with equal total protein loading without cellulolytic enhancing activity (1-50 mg of cellulolytic protein/g of cellulose in PCS).

AA9 polypeptide enhancing activity can be determined using a mixture of CELLUCLAST™ 1.5L (Novozymes A/S, Bagsvrd, Denmark) and beta-glucosidase as the source of the cellulolytic activity, wherein the beta-glucosidase is present at a weight of at least 2-5% protein of the cellulase protein loading. In one aspect, the beta-glucosidase is an Aspergillus oryzae beta-glucosidase (e.g., recombinantly produced in Aspergillus oryzae according to WO 02/095014). In another aspect, the beta-glucosidase is an Aspergillus fumigatus beta-glucosidase (e.g., recombinantly produced in Aspergillus oryzae as described in WO 02/095014).

AA9 polypeptide enhancing activity can also be determined by incubating an AA9 polypeptide with 0.5% phosphoric acid swollen cellulose (PASO), 100 mM sodium acetate pH 5, 1 mM MnSO₄, 0.1% gallic acid, 0.025 mg/ml of Aspergillus fumigatus beta-glucosidase, and 0.01% TRITON® X-100 (4-(1,1,3,3-tetramethylbutyl)phenyl-polyethylene glycol) for 24-96 hours at 40° C. followed by determination of the glucose released from the PASO.

AA9 polypeptide enhancing activity can also be determined according to WO 2013/028928 for high temperature compositions.

AA9 polypeptides enhance the hydrolysis of a cellulosic material catalyzed by enzyme having cellulolytic activity by reducing the amount of cellulolytic enzyme required to reach the same degree of hydrolysis preferably at least 1.01-fold, e.g., at least 1.05-fold, at least 1.10-fold, at least 1.25-fold, at least 1.5-fold, at least 2-fold, at least 3-fold, at least 4-fold, at least 5-fold, at least 10-fold, or at least 20-fold.

The AA9 polypeptide can also be used in the presence of a soluble activating divalent metal cation according to WO 2008/151043 or WO 2012/122518, e.g., manganese or copper.

The AA9 polypeptide can be used in the presence of a dioxy compound, a bicylic compound, a heterocyclic compound, a nitrogen-containing compound, a quinone compound, a sulfur-containing compound, or a liquor obtained from a pretreated cellulosic or hemicellulosic material such as pretreated corn stover (WO 2012/021394, WO 2012/021395, WO 2012/021396, WO 2012/021399, WO 2012/021400, WO 2012/021401, WO 2012/021408, and WO 2012/021410).

Beta-glucanase: The term “beta-glucanase” means a 3 (or 4)-beta-D-glucan 3(4)-glucanohydrolase that catalyzes the hydrolysis of (1,3)- or (1,4)-linkages in beta-D-glucans (E.C. 3.2.1.6) or a (1->3)-(1->4)-beta-D-glucan 4-glucanohydrolase that catalyzes hydrolysis of (1->4)-beta-D-glucosidic linkages in beta-D-glucans containing (1->3)- and (1->4)-bonds (E.C. 3.2.1.73). Beta-glucanase activity can be determined using carboxymethyl cellulose (CMC) as substrate according to the procedure of Ghose, 1987, Pure and Appl. Chem. 59: 257-268, at pH 5, 40° C.

Beta-glucosidase: The term “beta-glucosidase” means a beta-D-glucoside glucohydrolase (E.C. 3.2.1.21) that catalyzes the hydrolysis of terminal non-reducing beta-D-glucose residues with the release of beta-D-glucose. Beta-glucosidase activity can be determined using p-nitrophenyl-beta-D-glucopyranoside as substrate according to the procedure of Venturi et al., 2002, J. Basic Microbiol. 42: 55-66. One unit of beta-glucosidase is defined as 1.0 μmole of p-nitrophenolate anion produced per minute at 25° C., pH 4.8 from 1 mM p-nitrophenyl-beta-D-glucopyranoside as substrate in 50 mM sodium citrate containing 0.01% TWEEN® 20.

Beta-xylosidase: The term “beta-xylosidase” means a beta-D-xyloside xylohydrolase (E.C. 3.2.1.37) that catalyzes the exo-hydrolysis of short beta (1→4)-xylooligosaccharides to remove successive D-xylose residues from non-reducing termini. Beta-xylosidase activity can be determined using 1 mM p-nitrophenyl-beta-D-xyloside as substrate in 100 mM sodium citrate containing 0.01% TWEEN® 20 at pH 5, 40° C. One unit of beta-xylosidase is defined as 1.0 μmole of p-nitrophenolate anion produced per minute at 40° C., pH 5 from 1 mM p-nitrophenyl-beta-D-xyloside in 100 mM sodium citrate containing 0.01% TWEEN® 20.

Catalase: The term “catalase” means a hydrogen-peroxide:hydrogen-peroxide oxidoreductase (E.C. 1.11.1.6 or E.C. 1.11.1.21) that catalyzes the conversion of two hydrogen peroxides to oxygen and two waters.

Catalase activity can be determined by monitoring the degradation of hydrogen peroxide at 240 nm based on the following reaction:

2H₂O₂→2H₂O+O₂

The reaction is conducted in 50 mM phosphate pH 7 at 25° C. with 10.3 mM substrate (H₂O₂). Absorbance is monitored spectrophotometrically within 16-24 seconds, which should correspond to an absorbance reduction from 0.45 to 0.4. One catalase activity unit can be expressed as one μmole of H₂O₂ degraded per minute at pH 7.0 and 25° C.

Cellobiohydrolase: The term “cellobiohydrolase” means a 1,4-beta-D-glucan cellobiohydrolase (E.C. 3.2.1.91 and E.C. 3.2.1.176) that catalyzes the hydrolysis of 1,4-beta-D-glucosidic linkages in cellulose, cellooligosaccharides, or any beta-1,4-linked glucose containing polymer, releasing cellobiose from the reducing end (cellobiohydrolase I) or non-reducing end (cellobiohydrolase II) of the chain (Teeri, 1997, Trends in Biotechnology 15: 160-167; Teeri et al., 1998, Biochem. Soc. Trans. 26: 173-178). Cellobiohydrolase activity can be determined according to the procedures described by Lever et al., 1972, Anal. Biochem. 47: 273-279; van Tilbeurgh et al., 1982, FEBS Letters 149: 152-156; van Tilbeurgh and Claeyssens, 1985, FEBS Letters 187: 283-288; and Tomme et al., 1988, Eur. J. Biochem. 170: 575-581.

Cellulolytic enzyme composition: The term “cellulolytic enzyme composition” means an enzyme composition comprising a mixture of cellulolytic enzymes and accessory enzymes. The cellulolytic enzyme composition can be any of the enzyme compositions disclosed in WO 2013/028928, WO 2015/081139 and/or WO 2015/187935. The term “cellulolytic enzyme” or “cellulase” means one or more (e.g., several) enzymes that hydrolyze a cellulosic material. Such enzymes include endoglucanase(s), cellobiohydrolase(s), beta-glucosidase(s), or combinations thereof. The two basic approaches for measuring cellulolytic enzyme activity include: (1) measuring the total cellulolytic enzyme activity, and (2) measuring the individual cellulolytic enzyme activities (endoglucanases, cellobiohydrolases, and beta-glucosidases) as reviewed in Zhang et al., 2006, Biotechnology Advances 24: 452-481. Total cellulolytic enzyme activity can be measured using insoluble substrates, including Whatman No 1 filter paper, microcrystalline cellulose, bacterial cellulose, algal cellulose, cotton, pretreated lignocellulose, etc. The most common total cellulolytic activity assay is the filter paper assay using Whatman No 1 filter paper as the substrate. The assay was established by the International Union of Pure and Applied Chemistry (IUPAC) (Ghose, 1987, Pure Appl. Chem. 59: 257-68).

Cellulolytic enzyme activity can be determined by measuring the increase in production/release of sugars during hydrolysis of a cellulosic material by cellulolytic enzyme(s) under the following conditions: 1-50 mg of cellulolytic enzyme protein/g of cellulose in pretreated corn stover (PCS) (or other pretreated cellulosic material) for 3-7 days at a suitable temperature such as 40° C.-80° C., e.g., 40° C., 45° C., 50° C., 55° C., 60° C., 65° C., 70° C., 75° C., or 80° C., and a suitable pH, such as 4-9, e.g., 4.0, 4.5, 5.0, 5.5, 6.0, 6.5, 7.0, 7.5, 8.0, 8.5, or 9.0, compared to a control hydrolysis without addition of cellulolytic enzyme protein. Typical conditions are 1 ml reactions, washed or unwashed PCS, 5% insoluble solids (dry weight), 50 mM sodium acetate pH 5, 1 mM MnSO₄, 50° C., 55° C., or 60° C., 72 hours, sugar analysis by AMINEX® HPX-87H column chromatography (Bio-Rad Laboratories, Inc.).

Cellulosic material: The term “cellulosic material” means any material containing cellulose. The predominant polysaccharide in the primary cell wall of biomass is cellulose, the second most abundant is hemicellulose, and the third is pectin. The secondary cell wall, produced after the cell has stopped growing, also contains polysaccharides and is strengthened by polymeric lignin covalently cross-linked to hemicellulose. Cellulose is a homopolymer of anhydrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

Cellulose is generally found, for example, in the stems, leaves, hulls, husks, and cobs of plants or leaves, branches, and wood of trees. The cellulosic material can be, but is not limited to, agricultural residue, herbaceous material (including energy crops), municipal solid waste, pulp and paper mill residue, waste paper, and wood (including forestry residue) (see, for example, Wiselogel et al., 1995, in Handbook on Bioethanol (Charles E. Wyman, editor), pp. 105-118, Taylor & Francis, Washington D.C.; Wyman, 1994, Bioresource Technology 50: 3-16; Lynd, 1990, Applied Biochemistry and Biotechnology 24/25: 695-719; Mosier et al., 1999, Recent Progress in Bioconversion of Lignocellulosics, in Advances in Biochemical Engineering/Biotechnology, T. Scheper, managing editor, Volume 65, pp. 23-40, Springer-Verlag, New York). It is understood herein that the cellulose may be in the form of lignocellulose, a plant cell wall material containing lignin, cellulose, and hemicellulose in a mixed matrix. In one aspect, the cellulosic material is any biomass material. In another aspect, the cellulosic material is lignocellulose, which comprises cellulose, hemicelluloses, and lignin.

In an embodiment, the cellulosic material is agricultural residue, herbaceous material (including energy crops), municipal solid waste, pulp and paper mill residue, waste paper, or wood (including forestry residue).

In another embodiment, the cellulosic material is arundo, bagasse, bamboo, corn cob, corn fiber, corn stover, miscanthus, rice straw, sugar cane straw, switchgrass, or wheat straw.

In another embodiment, the cellulosic material is aspen, eucalyptus, fir, pine, poplar, spruce, or willow.

In another embodiment, the cellulosic material is algal cellulose, bacterial cellulose, cotton linter, filter paper, microcrystalline cellulose (e.g., AVICEL®), or phosphoric-acid treated cellulose.

In another embodiment, the cellulosic material is an aquatic biomass. As used herein the term “aquatic biomass” means biomass produced in an aquatic environment by a photosynthesis process. The aquatic biomass can be algae, emergent plants, floating-leaf plants, or submerged plants.

The cellulosic material may be used as is or may be subjected to pretreatment, using conventional methods known in the art, as described herein. In a preferred aspect, the cellulosic material is pretreated.

Dissolved Oxygen Saturation Level: The saturation level of oxygen is determined at the standard partial pressure (0.21 atmosphere) of oxygen. The saturation level at the standard partial pressure of oxygen is dependent on the temperature and solute concentrations. In an embodiment where the temperature during hydrolysis is 50° C., the saturation level would typically be in the range of 5-5.5 mg oxygen per kg slurry, depending on the solute concentrations. Hence, a concentration of dissolved oxygen of 0.5 to 10% of the saturation level at 50° C. corresponds to an amount of dissolved oxygen in a range from 0.025 ppm (0.5×5/100) to 0.55 ppm (10×5.5/100), such as, e.g., 0.05 to 0.165 ppm, and a concentration of dissolved oxygen of 10-70% of the saturation level at 50° C. corresponds to an amount of dissolved oxygen in a range from 0.50 ppm (10×5/100) to 3.85 ppm (70×5.5/100), such as, e.g., 1 to 2 ppm. In an embodiment, oxygen is added in an amount in the range of 0.5 to 5 ppm, such as 0.5 to 4.5 ppm, 0.5 to 4 ppm, 0.5 to 3.5 ppm, 0.5 to 3 ppm, 0.5 to 2.5 ppm, or 0.5 to 2 ppm.

Endoglucanase: The term “endoglucanase” means a 4-(1,3;1,4)-beta-D-glucan 4-glucanohydrolase (E.C. 3.2.1.4) that catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3-1,4 glucans such as cereal beta-D-glucans or xyloglucans, and other plant material containing cellulosic components. Endoglucanase activity can be determined by measuring reduction in substrate viscosity or increase in reducing ends determined by a reducing sugar assay (Zhang et al., 2006, Biotechnology Advances 24: 452-481). Endoglucanase activity can also be determined using carboxymethyl cellulose (CMC) as substrate according to the procedure of Ghose, 1987, Pure and Appl. Chem. 59: 257-268, at pH 5, 40° C.

Fragment: The term “fragment” means a polypeptide or a catalytic or binding domain having one or more (e.g., several) amino acids absent from the amino and/or carboxyl terminus of a mature polypeptide or domain; wherein the fragment has enzymatic or substrate binding activity.

Feruloyl esterase: The term “feruloyl esterase” means a 4-hydroxy-3-methoxycinnamoyl-sugar hydrolase (EC 3.1.1.73) that catalyzes the hydrolysis of 4-hydroxy-3-methoxycinnamoyl (feruloyl) groups from esterified sugar, which is usually arabinose in natural biomass substrates, to produce ferulate (4-hydroxy-3-methoxycinnamate). Feruloyl esterase (FAE) is also known as ferulic acid esterase, hydroxycinnamoyl esterase, FAE-III, cinnamoyl ester hydrolase, FAEA, cinnAE, FAE-I, or FAE-II. Feruloyl esterase activity can be determined using 0.5 mM p-nitrophenylferulate as substrate in 50 mM sodium acetate pH 5.0. One unit of feruloyl esterase equals the amount of enzyme capable of releasing 1 μmole of p-nitrophenolate anion per minute at pH 5, 25° C.

Hemicellulolytic enzyme composition: The term “hemicellulolytic enzyme composition” means an enzyme composition comprising a mixture of hemicellulolytic enzymes and accessory enzymes. In one embodiment the hemicellulolytic enzyme composition comprise a xylanase, an acetylxylan esterase, a feruloyl esterase, an arabinofuranosidase, a xylosidase, and a glucuronidase. In a preferred embodiment, the hemicellulolytic enzyme composition is Cellic® HTec3 or Cellic® HTec3.5 obtainable from Novozymes A/S. The term “hemicellulolytic enzyme” or “hemicellulase” means one or more (e.g., several) enzymes that hydrolyze a hemicellulosic material. See, for example, Shallom and Shoham, 2003, Current Opinion In Microbiology 6 (3): 219-228). Hemicellulases are key components in the degradation of plant biomass. Examples of hemicellulases include, but are not limited to, an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. The substrates for these enzymes, hemicelluloses, are a heterogeneous group of branched and linear polysaccharides that are bound via hydrogen bonds to the cellulose microfibrils in the plant cell wall, crosslinking them into a robust network. Hemicelluloses are also covalently attached to lignin, forming together with cellulose a highly complex structure. The variable structure and organization of hemicelluloses require the concerted action of many enzymes for its complete degradation. The catalytic modules of hemicellulases are either glycoside hydrolases (GHs) that hydrolyze glycosidic bonds, or carbohydrate esterases (CEs), which hydrolyze ester linkages of acetate or ferulic acid side groups. These catalytic modules, based on homology of their primary sequence, can be assigned into GH and CE families. Some families, with an overall similar fold, can be further grouped into clans, marked alphabetically (e.g., GH-A). A most informative and updated classification of these and other carbohydrate active enzymes is available in the Carbohydrate-Active Enzymes (CAZy) database. Hemicellulolytic enzyme activities can be measured according to Ghose and Bisaria, 1987, Pure & Appl. Chem. 59: 1739-1752, at a suitable temperature such as 40° C.-80° C., e.g., 40° C., 45° C., 50° C., 55° C., 60° C., 65° C., 70° C., 75° C., or 80° C., and a suitable pH such as 4-9, e.g., 4.0, 4.5, 5.0, 5.5, 6.0, 6.5, 7.0, 7.5, 8.0, 8.5, or 9.0.

Hemicellulosic material: The term “hemicellulosic material” means any material comprising hemicelluloses. Hemicelluloses include xylan, glucuronoxylan, arabinoxylan, glucomannan, and xyloglucan. These polysaccharides contain many different sugar monomers. Sugar monomers in hemicellulose can include xylose, mannose, galactose, rhamnose, and arabinose. Hemicelluloses contain most of the D-pentose sugars. Xylose is in most cases the sugar monomer present in the largest amount, although in softwoods mannose can be the most abundant sugar. Xylan contains a backbone of beta-(1-4)-linked xylose residues. Xylans of terrestrial plants are heteropolymers possessing a beta-(1-4)-D-xylopyranose backbone, which is branched by short carbohydrate chains. They comprise D-glucuronic acid or its 4-O-methyl ether, L-arabinose, and/or various oligosaccharides, composed of D-xylose, L-arabinose, D- or L-galactose, and D-glucose. Xylan-type polysaccharides can be divided into homoxylans and heteroxylans, which include glucuronoxylans, (arabino)glucuronoxylans, (glucurono)arabinoxylans, arabinoxylans, and complex heteroxylans. See, for example, Ebringerova et al., 2005, Adv. Polym. Sci. 186: 1-67. Hemicellulosic material is also known herein as “xylan-containing material”.

Sources for hemicellulosic material are essentially the same as those for cellulosic material described herein.

In the processes of the present invention, The CBC can further comprise hemicellulosic materials. any material containing hemicellulose may be used. In a preferred aspect, the hemicellulosic material is lignocellulose.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Lipase: The term “lipase” means a triacyl glycerol lipase (E.C.3.1.1.3), phospholipase A1 (EC 3.1.1.32) and phospholipase A2 (E.C.3.1.1.4), but also other phospholipases (E.C.3.1.1.5), (E.C.3.1.4.4), (E.C.3.1.4.11), (E.C.3.1.4.50), (E.C.3.1.4.54). The lipase activity can be determined according to EP0258068.

Mannanase: The term “mannanase” means a beta-mannanase belonging to EC 3.2.1.78 or E.C.3.2.1.25. Mannanases have been identified in several Bacillus organisms. For example, Talbot et al., Appl. Environ. Microbiol. Vol. 56, No. 11, pp. 3505-3510 (1990) describes a beta-mannanase derived from Bacillus stearothermophilus having an optimum pH of 5.5-7.5. Mendoza et al., World J. Microbiol. Biotech. Vol. 10, No. 5, pp. 551-555 (1994) describes a beta-mannanase derived from Bacillus subtilis having an optimum activity at pH 5.0 and 55° C. JP-03047076 discloses a beta-mannanase derived from Bacillus sp., having an optimum pH of 8-10. JP-63056289 describes the production of an alkaline, thermostable beta-mannanase. JP-08051975 discloses alkaline beta-mannanases from alkalophilic Bacillus sp. AM-001. A purified mannanase from Bacillus amyloiquefaciens is disclosed in WO 97/11164. WO 94/25576 discloses an enzyme from Aspergillus aculeatus, CBS 101.43, exhibiting mannanase activity and WO 93/24622 discloses a mannanase isolated from Trichoderma reesei. The mannanase may be derived from a strain of the genus Bacillus, such as the amino acid sequence having the sequence deposited as GENESEQP accession number AAY54122 or an amino acid sequence which is homologous to this amino acid sequence. The mannanase may be derived from a strain of the genus Talaromyces, such as Talaromyces Leycettanus, such as a mannanase disclosed in CN103425792 or an amino acid sequence which is homologous to such an amino acid sequence.

Mature polypeptide: The term “mature polypeptide” means a polypeptide in its final form following translation and any post-translational modifications, such as N-terminal processing, C-terminal truncation, glycosylation, phosphorylation, etc.

In one aspect, the mature polypeptide of the protease of SEQ ID NO: 1 is amino acids 1 to 177 of SEQ ID NO: 1 based on the SignalP 3.0 program (Bendtsen et al., 2004, J. Mol. Biol. 340: 783-795) that predicts amino acids −177 to −159 of SEQ ID NO: 1 are a signal peptide. In another aspect, the mature polypeptide of the protease of SEQ ID NO: 2 is amino acids 18 to 291 of SEQ ID NO: 2 based on the SignalP 3.0 program that predicts amino acids 1 to 17 of SEQ ID NO: 2 are a signal peptide. In another aspect, the mature polypeptide of the mannanas of SEQ ID NO: 3 is amino acids 2 to 302 of SEQ ID NO: 3 based on the SignalP 3.0 program. In another aspect, the mature polypeptide of SEQ ID NO: 4 is amino acids 40 to 245 of SEQ ID NO: 4 based on the SignalP 3.0 program. In another aspect, the mature polypeptide of SEQ ID NO: 5 is amino acids 1 to 412 of SEQ ID NO: 5 based on the SignalP 3.0 program. In another aspect, the mature polypeptide of the AA9 polypeptide of SEQ ID NO: 6 is amino acids 20 to 326 of SEQ ID NO: 6 based on the SignalP 3.0 program that predicts amino acids 1 to 19 of SEQ ID NO: 6 are a signal peptide. In another aspect, the mature polypeptide of the AA9 polypeptide of SEQ ID NO: 7 is amino acids 18 to 240 of SEQ ID NO: 7 based on the SignalP 3.0 program that predicts amino acids 1 to 17 of SEQ ID NO: 7 are a signal peptide. In another aspect, the mature polypeptide of the AA9 polypeptide of SEQ ID NO: 8 is amino acids 20 to 258 of SEQ ID NO: 8 based on the SignalP 3.0 program that predicts amino acids 1 to 19 of SEQ ID NO: 8 are a signal peptide. In another aspect, the mature polypeptide of the AA9 polypeptide of SEQ ID NO: 9 is amino acids 19 to 226 of SEQ ID NO: 9 based on the SignalP 3.0 program that predicts amino acids 1 to 18 of SEQ ID NO: 9 are a signal peptide. In another aspect, the mature polypeptide of the AA9 polypeptide of SEQ ID NO: 10 is amino acids 20 to 304 of SEQ ID NO: 10 based on the SignalP 3.0 program that predicts amino acids 1 to 19 of SEQ ID NO: 10 are a signal peptide. In another aspect, the mature polypeptide of the AA9 polypeptide of SEQ ID NO: 11 is amino acids 23 to 250 of SEQ ID NO: 11 based on the SignalP 3.0 program that predicts amino acids 1 to 22 of SEQ ID NO: 11 are a signal peptide. In another aspect, the mature polypeptide of the AA9 polypeptide of SEQ ID NO: 12 is amino acids 20 to 249 of SEQ ID NO: 12 based on the SignalP 3.0 program that predicts amino acids 1 to 19 of SEQ ID NO: 12 are a signal peptide. In another aspect, the mature polypeptide of the beta-glucosidase of SEQ ID NO: 13 is amino acids 22 to 1097 of SEQ ID NO: 13 based on the SignalP 3.0 program that predicts amino acids 1 to 21 of SEQ ID NO: 13 are a signal peptide. In another aspect, the mature polypeptide of the beta-glucosidase of SEQ ID NO: 14 is amino acids 22 to 1097 of SEQ ID NO: 14 based on the SignalP 3.0 program that predicts amino acids 1 to 21 of SEQ ID NO: 14 are a signal peptide. In another aspect, the mature polypeptide of the cellobiohydrolase I of SEQ ID NO: 15 is amino acids 27 to 532 of SEQ ID NO: 15 based on the SignalP 3.0 program that predicts amino acids 1 to 26 of SEQ ID NO: 15 are a signal peptide. In another aspect, the mature polypeptide of the cellobiohydrolase II of SEQ ID NO: 16 is amino acids 20 to 454 of SEQ ID NO: 16 based on the SignalP 3.0 program that predicts amino acids 1 to 19 of SEQ ID NO:

16 are a signal peptide. In another aspect, the mature polypeptide of the beta-glucosidase of SEQ ID NO: 17 is amino acids 20 to 863 of SEQ ID NO: 17 based on the SignalP 3.0 program that predicts amino acids 1 to 19 of SEQ ID NO: 17 are a signal peptide. In another aspect, the mature polypeptide of the AA9 polypeptide of SEQ ID NO: 18 is amino acids 26 to 253 of SEQ ID NO: 18 based on the SignalP 3.0 program that predicts amino acids 1 to 25 of SEQ ID NO: 18 are a signal peptide. In another aspect, the mature polypeptide of the xylanase of SEQ ID NO: 19 is amino acids 18 to 364 of SEQ ID NO: 19 based on the SignalP 3.0 program that predicts amino acids 1 to 17 of SEQ ID NO: 19 are a signal peptide. In another aspect, the mature polypeptide of the xylanase of SEQ ID NO: 20 is amino acids 20 to 323 of SEQ ID NO: 20 based on the SignalP 3.0 program that predicts amino acids 1 to 19 of SEQ ID NO: 20 are a signal peptide. In another aspect, the mature polypeptide of the xylanase of SEQ ID NO: 21 is amino acids 20 to 397 of SEQ ID NO: 21 based on the SignalP 3.0 program that predicts amino acids 1 to 19 of SEQ ID NO: 21 are a signal peptide. In another aspect, the mature polypeptide of the beta-xylosidase of SEQ ID NO: 22 is amino acids 21 to 792 of SEQ ID NO: 22 based on the SignalP 3.0 program that predicts amino acids 1 to 20 of SEQ ID NO: 22 are a signal peptide. In another aspect, the mature polypeptide of the xylanase of SEQ ID NO: 23 is amino acids 20 to 398 of SEQ ID NO: 23 based on the SignalP 3.0 program that predicts amino acids 1 to 19 of SEQ ID NO: 23 are a signal peptide. In another aspect, the mature polypeptide of the beta-xylosidase of SEQ ID NO: 24 is amino acids 22 to 796 of SEQ ID NO: 24 based on the SignalP 3.0 program that predicts amino acids 1 to 21 of SEQ ID NO: 24 are a signal peptide. In another aspect, the mature polypeptide of the cellobiohydrolase I of SEQ ID NO: 25 is amino acids 26 to 532 of SEQ ID NO: 25 based on the SignalP 3.0 program (that predicts amino acids 1 to 25 of SEQ ID NO: 25 are a signal peptide. In another aspect, the mature polypeptide of a cellobiohydrolase II of SEQ ID NO: 26 is amino acids 19 to 464 of SEQ ID NO: 26 based on the SignalP 3.0 program that predicts amino acids 1 to 18 of SEQ ID NO: 26 are a signal peptide. In another aspect, the mature polypeptide of the xylanase of SEQ ID NO: 27 is amino acids 21 to 405 of SEQ ID NO: 27 based on the SignalP 3.0 program that predicts amino acids 1 to 20 of SEQ ID NO: 27 are a signal peptide. In another aspect, the mature polypeptide of the xylanase of SEQ ID NO: 28 is amino acids 20 to 398 of SEQ ID NO: 28 based on the SignalP 3.0 program that predicts amino acids 1 to 19 of SEQ ID NO: 28 are a signal peptide. In another aspect, the mature polypeptide of the endoglucanase I of SEQ ID NO: 29 is amino acids 23 to 459 of SEQ ID NO: 29 based on the SignalP 3.0 program that predicts amino acids 1 to 22 of SEQ ID NO: 29 are a signal peptide. In another aspect, the mature polypeptide of the endoglucanase II of SEQ ID NO: 30 is amino acids 22 to 418 of SEQ ID NO: 30 based on the SignalP 3.0 program that predicts amino acids 1 to 21 of SEQ ID NO: 30are a signal peptide. In another aspect, the mature polypeptide of the endoglucanase II of SEQ ID NO: 31 is amino acids 19 to 335 of SEQ ID NO: 31 based on the SignalP 3.0 program that predicts amino acids 1 to 18 of SEQ ID NO: 31 are a signal peptide. In another aspect, the mature polypeptide of the protease of SEQ ID NO: 32 is amino acids 1 to 269 of SEQ ID NO: 32 based on the SignalP 3.0 program. In another aspect, the mature polypeptide of the protease of SEQ ID NO: 33 is amino acids 1 to 274 of SEQ ID NO: 33 based on the SignalP 3.0 program. In another aspect, the mature polypeptide of the lipase of SEQ ID NO: 35 is amino acids 1 to 274 of SEQ ID NO: 35 based on the SignalP 3.0 program. In another aspect, the mature polypeptide of the protease of SEQ ID NO: 36 is amino acids 199 to 564 of SEQ ID NO: 36 based on the SignalP 3.0 program that predicts amino acids 1 to 17 of SEQ ID NO: 36 are a signal peptide.

Municipal solid waste or MSW: The term “municipal solid waste” or “MSW” means solid waste fractions that are typically available in municipalities (cities, towns, villages). MSW can be a combination of plant materials (fruit, vegetables, grains, corn etc.), animal materials (meats etc.), cellulosic material (paper, cardboard, diapers, textile etc.), glass, plastic, metal, etc., which can be combined with various fractions at any ratios. MSW includes, but is not limited to, any one or more of the following: waste collected from homes, schools, hospitals, offices, business, industries such as restaurants and food processing industries. MSW can potentially have been treated by shredding or pulping devices. Examples of model MSW substrates are provided in Example 1 herein.

Pectinase: The term pectinase or pectolytic enzyme is intended to include any pectinase enzyme defined according to the art where pectinases are a group of enzymes that catalyze the cleavage of glycosidic linkages. Basically three types of pectolytic enzymes exist: pectinesterase, which only removes methoxyl residues from pectin, a range of depolymerizing enzymes, and protopectinase, which solubilizes protopectin to form pectin (Sakai et al., (1993) Advances in Applied Microbiology vol 39 pp 213-294). Example of a pectinases or pectolytic enzyme useful in the invention is pectate lyase (EC 4.2.2.2 and EC 4.2.2.9), polygalacturonase (EC 3.2.1.15 and EC 3.2.1.67), polymethyl galacturonase, pectin lyase (EC 4.2.2.10), galactanases (EC 3.2.1.89), arabinanases (EC 3.2.1.99) and/or pectin esterases (EC 3.1.1.11).

Suitable pectinolytic enzymes include those described in WO 99/27083, WO 99/27084, WO 00/55309, WO 02/092741 and WO08039353.

Pretreated cellulosic or hemicellulosic material: The term “pretreated cellulosic or hemicellulosic material” means a cellulosic or hemicellulosic material derived from pretreatment with heat and dilute sulfuric acid, alkaline pretreatment, neutral pretreatment, or any pretreatment known in the art.

Pretreated municipal solid waste material: The term “pretreated municipal solid waste material” means a municipal solid waste material derived from pretreatment with heat and dilute sulfuric acid, alkaline pretreatment, neutral pretreatment, or any pretreatment known in the art.

### Protease

Protease: The term “protease” means an enzyme that hydrolyses peptide bonds. It includes any enzymes belonging to the EC 3.4 enzyme group (including each of the thirteen subclasses thereof) of the EC list (Enzyme Nomenclature 1992 from NC-IUBMB, Academic Press, San Diego, Calif.) as regularly supplemented and updated, see e.g. the World Wide Web (WWW) at http://www.chem.qmw.ac.uk/iubmb/enzyme/index.html.

Proteases are classified on the basis of their catalytic mechanism into the following groups: Serine proteases (S), Cysteine proteases (C), Aspartic proteases (A), Metalloproteases (M), and Unknown, or as yet unclassified, proteases (U), see Handbook of Proteolytic Enzymes, A. J. Barrett, N. D. Rawlings, J. F. Woessner (eds), Academic Press (1998), in particular the general introduction part.

WO 97/46689 discloses the protease (encoding) sequences from specific Aspergillus strains. WO 2003/048353 disclosed the protease derived from a fungus of the species Thermoascus aurantiacus. The entire disclosure of the above mentioned application is incorporated herein.

In one embodiment of the present invention, the protease is derived from a fungus of the genus Thermoascus, for example the species Thermoascus aurantiacus, such as the strain Thermoascus aurantiacus CGMCC No. 0582, e.g., a polypeptide with the amino acid sequence of amino acids −178 to 177, −159 to 177, or +1 to 177 of SEQ ID NO: 1. The process for preparing such protease is disclosed in WO 2003/048353.

In another embodiment of the present invention, the protease is derived from a strain of Pyrococcus furiosus, in particular the one shown in SEQ ID NO: 6 herein or disclosed in U.S. Pat. No. 6,358,726-B1.

In another embodiment of the present invention, the protease is derived from a strain of Bacillus clausii, in particular the one shown in SEQ ID NO:32, SEQ ID NO: 32+(Y161A+R164S+A188P); SEQ ID NO: 32+(M216S); SEQ ID NO: 32+S97AD (insertion of D and substitution of S with A at position 97 in the back bone) or SEQ ID NO: 32+(V66A+S104A), as disclosed in WO 2011/036263, WO 1998/020115 and WO 2003/006602;

In another embodiment of the present invention, the protease is derived from a strain of Bacillus licheniformis, in particular, the one shown in SEQ ID NO:33 herein or disclosed in WO2015/144936 and WO2015/144782;

In still another embodiment of the present invention, the protease is derived from a strain of Meripilus giganteus, in particular, a polypeptide with the amino acid sequence of amino acids 1-564, 18-564, or 199-564 of SEQ ID NO:36 herein or disclosed in WO2014/037438.

In an embodiment the protease has at least 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or at least 100% of the identity with the mature polypeptides of SEQ ID NO: 1, 5, 32, 33, or 36 of the present invention.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Solubilization/Hydrolysis: In the solubilization step, the municipal solid waste material, e.g., pretreated, sorted or unsorted, is hydrolyzed to break down cellulose and/or hemicellulose and other substrates. The material may be hydrolyzed to fermentable sugars, such as glucose, cellobiose, xylose, xylulose, arabinose, mannose, galactose, and/or soluble oligosaccharides (also known as saccharification). In a solubilization process the material is solubilized to a bioliquid comprising the solubilized material. The hydrolysis is performed enzymatically by one or more enzyme compositions in one or more stages. In the hydrolysis step, the municipal solid waste material, e.g., pretreated, is hydrolyzed to break down proteins and lipids (e.g. triglycerides) found in the waste. The solubilization and/or hydrolysis can be carried out as a batch process or series of batch processes. The solubilization and/or hydrolysis can be carried out as a fed batch or continuous process, or series of fed batch or continuous processes, where the solid waste is fed gradually to, for example, a hydrolysis solution containing an enzyme composition. In an embodiment, the solubilization and/or hydrolysis is a continuous process in which a solid waste and a cellulolytic enzyme composition are added at different intervals throughout the process and the hydrolysate is removed at different intervals throughout the process. In one embodiment, the municipal solid waste is sorted before solubilization, then separated. In another embodiment, the municipal solid waste unsorted before solubilization is separated after solubilization.

Variant: The term “variant” means a polypeptide comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position.

Xylanase: The term “xylanase” means a 1,4-beta-D-xylan-xylohydrolase (E.C. 3.2.1.8) that catalyzes the endohydrolysis of 1,4-beta-D-xylosidic linkages in xylans. Xylanase activity can be determined with 0.2% AZCL-arabinoxylan as substrate in 0.01% TRITON® X-100 and 200 mM sodium phosphate pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6.

Reference to “about” a value or parameter herein includes aspects that are directed to that value or parameter per se. For example, description referring to “about X” includes the aspect “X”.

As used herein and in the appended claims, the singular forms “a,” “or,” and “the” include plural referents unless the context clearly dictates otherwise. It is understood that the aspects of the invention described herein include “consisting” and/or “consisting essentially of” aspects.

Unless defined otherwise or clearly indicated by context, all technical and scientific terms used herein have the same meaning as commonly understood by one of ordinary skill in the art to which this invention belongs.

### DETAILED DESCRIPTION OF THE INVENTION

The present invention relates to processes for solubilizing a municipal solid waste, comprising treating the municipal solid waste with an enzyme composition comprising (i) a cellulolytic enzyme composition and (ii) a protease a protease selected from the group consisting of: (a) a protease having at least 50%, 55%, 60%, 65%, 70%, 75%, 80%, 85%, 90%, 95% or 100% identity to amino acids 1 to 177 of SEQ ID NO: 1 or a fragment thereof having protease activity; (b) a protease having at least 50%, 55%, 60%, 65%, 70%, 75%, 80%, 85%, 90%, 95% or 100% identity to amino acids of SEQ ID NO: 5; (c) a protease having at least 50%, 55%, 60%, 65%, 70%, 75%, 80%, 85%, 90%, 95% or 100% identity to amino acids of SEQ ID NO: 32 or a variant thereof; (d) a protease having at least 50%, 55%, 60%, 65%, 70%, 75%, 80%, 85%, 90%, 95% or 100% identity to amino acids of SEQ ID NO: 33; and (e) a protease having at least 50%, 55%, 60%, 65%, 70%, 75%, 80%, 85%, 90%, 95% or 100% identity to amino acids 199 to 564 of SEQ ID NO: 36; effective to produce a solubilized municipal solid waste. In one aspect, the processes further comprise recovering the solubilized municipal solid waste.

The present invention also relates to processes for producing a fermentation product, comprising: (a) solubilizing a municipal solid waste with an effective amount of enzyme composition comprising (i) a cellulolytic enzyme composition and (ii) a protease a protease selected from the group consisting of: (a) a protease having at least 50%, 55%, 60%, 65%, 70%, 75%, 80%, 85%, 90%, 95% or 100% identity to amino acids 1 to 177 of SEQ ID NO: 1 or a fragment thereof having protease activity; (b) a protease having at least 50%, 55%, 60%, 65%, 70%, 75%, 80%, 85%, 90%, 95% or 100% identity to amino acids of SEQ ID NO: 5; (c) a protease having at least 50%, 55%, 60%, 65%, 70%, 75%, 80%, 85%, 90%, 95% or 100% identity to amino acids of SEQ ID NO: 32 or a variant thereof; (d) a protease having at least 50%, 55%, 60%, 65%, 70%, 75%, 80%, 85%, 90%, 95% or 100% identity to amino acids of SEQ ID NO: 33; and (e) a protease having at least 50%, 55%, 60%, 65%, 70%, 75%, 80%, 85%, 90%, 95% or 100% identity to amino acids 199 to 564 of SEQ ID NO: 36; (b) fermenting the solubilized municipal solid waste with one or more (e.g., several) fermenting microorganisms to produce the fermentation product; and (c) recovering the fermentation product from the fermentation.

The present invention also relates to processes of fermenting a municipal solid waste, comprising: fermenting the municipal solid waste with one or more (e.g., several) fermenting microorganisms, wherein the municipal solid waste is solubilized with an enzyme composition comprising (i) a cellulolytic enzyme composition and (ii) a protease a protease selected from the group consisting of: (a) a protease having at least 50%, 55%, 60%, 65%, 70%, 75%, 80%, 85%, 90%, 95% or 100% identity to amino acids 1 to 177 of SEQ ID NO: 1 or a fragment thereof having protease activity; (b) a protease having at least 50%, 55%, 60%, 65%, 70%, 75%, 80%, 85%, 90%, 95% or 100% identity to amino acids of SEQ ID NO: 5; (c) a protease having at least 50%, 55%, 60%, 65%, 70%, 75%, 80%, 85%, 90%, 95% or 100% identity to amino acids of SEQ ID NO: 32 or a variant thereof; (d) a protease having at least 50%, 55%, 60%, 65%, 70%, 75%, 80%, 85%, 90%, 95% or 100% identity to amino acids of SEQ ID NO: 33; and (e) a protease having at least 50%, 55%, 60%, 65%, 70%, 75%, 80%, 85%, 90%, 95% or 100% identity to amino acids 199 to 564 of SEQ ID NO: 36. In one aspect, the fermenting of the municipal solid waste produces a fermentation product. In another aspect, the processes further comprise recovering the fermentation product from the fermentation.

The present invention also relates to enzyme compositions comprising (i) a cellulolytic enzyme composition and (ii) a protease a protease selected from the group consisting of: (a) a protease having at least 50%, 55%, 60%, 65%, 70%, 75%, 80%, 85%, 90%, 95% or 100% identity to amino acids 1 to 177 of SEQ ID NO: 1 or a fragment thereof having protease activity; (b) a protease having at least 50%, 55%, 60%, 65%, 70%, 75%, 80%, 85%, 90%, 95% or 100% identity to amino acids of SEQ ID NO: 5; (c) a protease having at least 50%, 55%, 60%, 65%, 70%, 75%, 80%, 85%, 90%, 95% or 100% identity to amino acids of SEQ ID NO: 32 or a variant thereof; (d) a protease having at least 50%, 55%, 60%, 65%, 70%, 75%, 80%, 85%, 90%, 95% or 100% identity to amino acids of SEQ ID NO: 33; and (e) a protease having at least 50%, 55%, 60%, 65%, 70%, 75%, 80%, 85%, 90%, 95% or 100% identity to amino acids 199 to 564 of SEQ ID NO: 36. In one embodiment, the enzyme compositions further or even further comprise a xylanase, a beta-xylosidase, or a xylanase and a beta-xylosidase. In another embodiment, the enzyme compositions further or even further comprise one or more enzymes selected from a lipase, a mannanase, a pectinase, and a beta-glucanase.

In one embodiment, the protease is selected from the group consisting of: (a) a protease having at least 50%, 55%, 60%, 65%, 70%, 75%, 80%, 85%, 90%, 95% or 100% identity to amino acids 1 to 177 of SEQ ID NO: 1 or a fragment thereof having protease activity; (b) a protease having at least 50%, 55%, 60%, 65%, 70%, 75%, 80%, 85%, 90%, 95% or 100% identity to amino acids of SEQ ID NO: 5; (c) a protease having at least 50%, 55%, 60%, 65%, 70%, 75%, 80%, 85%, 90%, 95% or 100% identity to amino acids of SEQ ID NO: 32 or a variant thereof; (d) a protease having at least 50%, 55%, 60%, 65%, 70%, 75%, 80%, 85%, 90%, 95% or 100% identity to amino acids of SEQ ID NO: 33; and (e) a protease having at least 50%, 55%, 60%, 65%, 70%, 75%, 80%, 85%, 90%, 95% or 100% identity to amino acids 199 to 564 of SEQ ID NO: 36.

In one embodiment, the protease is selected from the group consisting of: (a) a protease having at least 50%, 55%, 60%, 65%, 70%, 75%, 80%, 85%, 90%, 95% or 100% identity to amino acids 1 to 177 of SEQ ID NO: 1 or a fragment thereof having protease activity; (b) a protease having at least 50%, 55%, 60%, 65%, 70%, 75%, 80%, 85%, 90%, 95% or 100% identity to amino acids of SEQ ID NO: 5; (c) a protease having at least 50%, 55%, 60%, 65%, 70%, 75%, 80%, 85%, 90%, 95% or 100% identity to amino acids of SEQ ID NO: 32 or a variant thereof; (d) a protease having at least 50%, 55%, 60%, 65%, 70%, 75%, 80%, 85%, 90%, 95% or 100% identity to amino acids of SEQ ID NO: 33; and (e) a protease having at least 50%, 55%, 60%, 65%, 70%, 75%, 80%, 85%, 90%, 95% or 100% identity to SEQ ID NO: 36 or a fragment thereof having protease activity.

The present invention is based on a surprising discovery that the protease having at least 50%, 55%, 60%, 65%, 70%, 75%, 80%, 85%, 90%, 95% or 100% identity with the amino acids 1 to 177 of SEQ ID NO: 1 or a fragment thereof having protease activity, the protease having at least 50%, 55%, 60%, 65%, 70%, 75%, 80%, 85%, 90%, 95% or 100% identity to amino acids of SEQ ID NO: 5; the protease having at least 50%, 55%, 60%, 65%, 70%, 75%, 80%, 85%, 90%, 95% or 100% identity to amino acids of SEQ ID NO: 32 or a variant thereof; a protease having at least 50%, 55%, 60%, 65%, 70%, 75%, 80%, 85%, 90%, 95% or 100% identity to amino acids of SEQ ID NO: 33; or a protease having at least 50%, 55%, 60%, 65%, 70%, 75%, 80%, 85%, 90%, 95% or 100% identity to SEQ ID NO: 36 or a fragment thereof having protease activity, are effective when mixed with a cellulolytic enzyme composition in solubilizing a municipal solid waste compared to the cellulolytic enzyme composition without the protease, and the amount of enzyme used to achieve the desirable solubilization is significantly reduced.

In embodiments of the invention enzymatic solubilization of municipal solid waste is carried out together with natural occurring microorganisms found in the waste (concurrent enzymatic and microbial hydrolysis and fermentation) or found in recycled process wastes/solutions.

In some embodiments the microbial growth has a pH lowering effect especially when metabolites like carboxylic acids and fatty acids (e.g. acetate, propionate, butyrate, lactate) are produced.

In other embodiments of the invention it might be an advantage to inoculate MSW using different microbial species. These might include microorganisms that produce extra-cellular cellulase activities, microorganisms capable of degrading lignin, acetate-producing microorganisms, propionate-producing microorganisms, butyrate-producing organisms, ethanol-producing microorganisms and lactate producing microorganisms. Such embodiments are further described on pages 21-25 of WO2013/185777, which is incorporated herein by reference in its entirety.

In the processes of the present invention, it can be advantageous to adjust temperature and water and dry matter content of the MSW. Enzymes normally show an optimal temperature and dry matter range. Hydrolysis of MSW is normally performed with agitation. This can be in reactors providing agitation by free fall mixing (as also described by WO2006/056838 and WO2011/032557), stirred-tank reactors or similar systems. Suitable process time, temperature and pH conditions can readily be determined by one skilled in the art and is dependent on MSW composition, dry matter concentration and enzyme.

Soluble products from the solubilized municipal solid waste can be separated from insolubilized municipal solid waste using methods known in the art such as, for example, centrifugation, filtration, or gravity settling. The soluble products can be converted to many useful products. In one embodiment, the soluble products can be converted to biogas (consisting mainly of CH₄ and CO₂) by an aerobic digestion. In another embodiment, solubilized sugars can be converted by fermentation to for example, fuel (ethanol, n-butanol, isobutanol, biodiesel, jet fuel) and/or platform chemicals (e.g., acids, alcohols, ketones, gases, oils, and the like). The production of a desired product from the municipal solid waste may involve pretreatment, enzymatic solubilization, and fermentation

The processing of the municipal solid waste material according to the present invention can be accomplished using methods conventional in the art. Moreover, the processes of the present invention can be implemented using any conventional biomass processing apparatus configured to operate in accordance with the invention.

Solubilization and/or hydrolysis and fermentation, separate or simultaneous, include, but are not limited to, separate hydrolysis and fermentation (SHF); simultaneous saccharification and fermentation (SSF); simultaneous saccharification and co-fermentation (SSCF); hybrid hydrolysis and fermentation (HHF); separate hydrolysis and co-fermentation (SHCF); hybrid hydrolysis and co-fermentation (HHCF); and direct microbial conversion (DMC), also sometimes called consolidated bioprocessing (CBP). SHF uses separate process steps to first enzymatically hydrolyze the municipal solid waste material to fermentable sugars, e.g., glucose, cellobiose, and pentose monomers, and then ferment the fermentable sugars to ethanol. In SSF, the enzymatic hydrolysis of the municipal solid waste material and the fermentation of sugars to ethanol are combined in one step (Philippidis, G. P., 1996, Cellulose bioconversion technology, in Handbook on Bioethanol: Production and Utilization, Wyman, C. E., ed., Taylor & Francis, Washington, D.C., 179-212). SSCF involves the co-fermentation of multiple sugars (Sheehan and Himmel, 1999, Biotechnol. Prog. 15: 817-827). HHF involves a separate hydrolysis step, and in addition a simultaneous saccharification and hydrolysis step, which can be carried out in the same reactor. The steps in an HHF process can be carried out at different temperatures, i.e., high temperature enzymatic saccharification followed by SSF at a lower temperature that the fermentation strain can tolerate. DMC combines all three processes (enzyme production, hydrolysis, and fermentation) in one or more (e.g., several) steps where the same organism is used to produce the enzymes for conversion of the municipal solid waste material to fermentable sugars and to convert the fermentable sugars into a final product (Lynd et al., 2002, Microbiol. Mol. Biol. Reviews 66: 506-577). It is understood herein that any method known in the art comprising pretreatment, enzymatic hydrolysis, fermentation, or a combination thereof, can be used in the practicing the processes of the present invention.

A conventional apparatus can include a fed-batch stirred reactor, a batch stirred reactor, a continuous flow stirred reactor with ultrafiltration, and/or a continuous plug-flow column reactor (de Castilhos Corazza et al., 2003, Acta Scientiarum. Technology 25: 33-38; Gusakov and Sinitsyn, 1985, Enz. Microb. Technol. 7: 346-352), an attrition reactor (Ryu and Lee, 1983, Biotechnol. Bioeng. 25: 53-65). Additional reactor types include fluidized bed, upflow blanket, immobilized, and extruder type reactors for hydrolysis and/or fermentation.

Pretreatment. In practicing the processes of the present invention, any pretreatment process known in the art can be used to disrupt plant cell wall components of the municipal solid waste material (Chandra et al., 2007, Adv. Biochem. Engin./Biotechnol. 108: 67-93; Galbe and Zacchi, 2007, Adv. Biochem. Engin./Biotechnol. 108: 41-65; Hendriks and Zeeman, 2009, Bioresource Technology 100: 10-18; Mosier et al., 2005, Bioresource Technology 96: 673-686; Taherzadeh and Karimi, 2008, Int. J. Mol. Sci. 9: 1621-1651; Yang and Wyman, 2008, Biofuels Bioproducts and Biorefining-Biofpr. 2: 26-40).

In a preferred embodiment of the invention MSW is subject to a mild to severe temperature pretreatment in the range 10-300° C. prior to hydrolysis. Heating will normally occur together with a mixing. Heating will normally be carried out by addition of water or steam. Pretreatment might also consist of a separation (manual or automatic) of MSW in different fractions. The municipal solid waste material can also be subjected to particle size reduction, sieving, pre-soaking, wetting, washing, and/or conditioning prior to pretreatment using methods known in the art.

Conventional pretreatments include, but are not limited to, steam pretreatment (with or without explosion), dilute acid pretreatment, hot water pretreatment, alkaline pretreatment, lime pretreatment, wet oxidation, wet explosion, ammonia fiber explosion, organosolv pretreatment, and biological pretreatment. Additional pretreatments include ammonia percolation, ultrasound, electroporation, microwave, supercritical CO₂, supercritical H₂O, ozone, ionic liquid, and gamma irradiation pretreatments.

The municipal solid waste material can be pretreated before hydrolysis and/or fermentation. Pretreatment is preferably performed prior to the hydrolysis. Alternatively, the pretreatment can be carried out simultaneously with enzyme hydrolysis to release fermentable sugars, such as glucose, xylose, and/or cellobiose. In most cases the pretreatment step itself results in some conversion of biomass to fermentable sugars (even in absence of enzymes).

Steam Pretreatment. In steam pretreatment, the municipal solid waste material is heated to disrupt the plant cell wall components, including lignin, hemicellulose, and cellulose to make the cellulose and other fractions, e.g., hemicellulose, accessible to enzymes. The municipal solid waste is passed to or through a reaction vessel where steam is injected to increase the temperature to the required temperature and pressure and is retained therein for the desired reaction time. Steam pretreatment is preferably performed at 140-250° C., e.g., 160-200° C. or 170-190° C., where the optimal temperature range depends on optional addition of a chemical catalyst. Residence time for the steam pretreatment is preferably 1-60 minutes, e.g., 1-30 minutes, 1-20 minutes, 3-12 minutes, or 4-10 minutes, where the optimal residence time depends on the temperature and optional addition of a chemical catalyst. Steam pretreatment allows for relatively high solids loadings, so that the municipal solid waste is generally only moist during the pretreatment. The steam pretreatment is often combined with an explosive discharge of the material after the pretreatment, which is known as steam explosion, that is, rapid flashing to atmospheric pressure and turbulent flow of the material to increase the accessible surface area by fragmentation (Duff and Murray, 1996, Bioresource Technology 855: 1-33; Galbe and Zacchi, 2002, Appl. Microbiol. Biotechnol. 59: 618-628; U.S. Patent Application No. 2002/0164730). During steam pretreatment, hemicellulose acetyl groups are cleaved and the resulting acid autocatalyzes partial hydrolysis of the hemicellulose to monosaccharides and oligosaccharides. Lignin is removed to only a limited extent.

Chemical Pretreatment. The term “chemical treatment” refers to any chemical pretreatment that promotes the separation and/or release of cellulose, hemicellulose, and/or lignin. Such a pretreatment can convert crystalline cellulose to amorphous cellulose. Examples of suitable chemical pretreatment processes include, for example, dilute acid pretreatment, lime pretreatment, wet oxidation, ammonia fiber/freeze expansion (AFEX), ammonia percolation (APR), ionic liquid, and organosolv pretreatments.

A chemical catalyst such as H₂SO₄ or SO₂ (typically 0.3 to 5% w/w) is sometimes added prior to steam pretreatment, which decreases the time and temperature, increases the recovery, and improves enzymatic hydrolysis (Ballesteros et al., 2006, Appl. Biochem. Biotechnol. 129-132: 496-508; Varga et al., 2004, Appl. Biochem. Biotechnol. 113-116: 509-523; Sassner et al., 2006, Enzyme Microb. Technol. 39: 756-762). In dilute acid pretreatment, the cellulosic or hemicellulosic material is mixed with dilute acid, typically H₂SO₄, and water to form a slurry, heated by steam to the desired temperature, and after a residence time flashed to atmospheric pressure. The dilute acid pretreatment can be performed with a number of reactor designs, e.g., plug-flow reactors, counter-current reactors, or continuous counter-current shrinking bed reactors (Duff and Murray, 1996, Bioresource Technology 855: 1-33; Schell et al., 2004, Bioresource Technology 91: 179-188; Lee et al., 1999, Adv. Biochem. Eng. Biotechnol. 65: 93-115).

Several methods of pretreatment under alkaline conditions can also be used. These alkaline pretreatments include, but are not limited to, sodium hydroxide, lime, wet oxidation, ammonia percolation (APR), and ammonia fiber/freeze expansion (AFEX) pretreatment.

Lime pretreatment is performed with calcium oxide or calcium hydroxide at temperatures of 85-150° C. and residence times from 1 hour to several days (Wyman et al., 2005, Bioresource Technology 96: 1959-1966; Mosier et al., 2005, Bioresource Technology 96: 673-686). WO 2006/110891, WO 2006/110899, WO 2006/110900, and WO 2006/110901 disclose pretreatment methods using ammonia.

Wet oxidation is a thermal pretreatment performed typically at 180-200° C. for 5-15 minutes with addition of an oxidative agent such as hydrogen peroxide or over-pressure of oxygen (Schmidt and Thomsen, 1998, Bioresource Technology 64: 139-151; Palonen et al., 2004, Appl. Biochem. Biotechnol. 117: 1-17; Varga et al., 2004, Biotechnol. Bioeng. 88: 567-574; Martin et al., 2006, J. Chem. Technol. Biotechnol. 81: 1669-1677). The pretreatment is performed preferably at 1-40% dry matter, e.g., 2-30% dry matter or 5-20% dry matter, and often the initial pH is increased by the addition of alkali such as sodium carbonate.

A modification of the wet oxidation pretreatment method, known as wet explosion (combination of wet oxidation and steam explosion) can handle dry matter up to 30%. In wet explosion, the oxidizing agent is introduced during pretreatment after a certain residence time. The pretreatment is then ended by flashing to atmospheric pressure (WO 2006/032282).

Ammonia fiber expansion (AFEX) involves treating the municipal solid waste with liquid or gaseous ammonia at moderate temperatures such as 90-150° C. and high pressure such as 17-20 bar for 5-10 minutes, where the dry matter content can be as high as 60% (Gollapalli et al., 2002, Appl. Biochem. Biotechnol. 98: 23-35; Chundawat et al., 2007, Biotechnol. Bioeng. 96: 219-231; Alizadeh et al., 2005, Appl. Biochem. Biotechnol. 121: 1133-1141; Teymouri et al., 2005, Bioresource Technology 96: 2014-2018). During AFEX pretreatment cellulose and hemicelluloses remain relatively intact. Lignin-carbohydrate complexes are cleaved.

Organosolv pretreatment delignifies the municipal solid waste by extraction using aqueous ethanol (40-60% ethanol) at 160-200° C. for 30-60 minutes (Pan et al., 2005, Biotechnol. Bioeng. 90: 473-481; Pan et al., 2006, Biotechnol. Bioeng. 94: 851-861; Kurabi et al., 2005, Appl. Biochem. Biotechnol. 121: 219-230). Sulphuric acid is usually added as a catalyst. In organosolv pretreatment, the majority of hemicellulose and lignin is removed.

Other examples of suitable pretreatment methods are described by Schell et al., 2003, Appl. Biochem. Biotechnol. 105-108: 69-85, and Mosier et al., 2005, Bioresource Technology 96: 673-686, and U.S. Published Application 2002/0164730.

In one aspect, the chemical pretreatment is preferably carried out as a dilute acid treatment, and more preferably as a continuous dilute acid treatment. The acid is typically sulfuric acid, but other acids can also be used, such as acetic acid, citric acid, nitric acid, phosphoric acid, tartaric acid, succinic acid, hydrogen chloride, or mixtures thereof. Mild acid treatment is conducted in the pH range of preferably 1-5, e.g., 1-4 or 1-2.5. In one aspect, the acid concentration is in the range from preferably 0.01 to 10 wt. % acid, e.g., 0.05 to 5 wt. % acid or 0.1 to 2 wt. % acid. The acid is contacted with the cellulosic or hemicellulosic material and held at a temperature in the range of preferably 140-200° C., e.g., 165-190° C., for periods ranging from 1 to 60 minutes.

In another aspect, pretreatment takes place in an aqueous slurry. In preferred aspects, the cellulosic or hemicellulosic material is present during pretreatment in amounts preferably between 10-80 wt. %, e.g., 20-70 wt. % or 30-60 wt. %, such as around 40 wt. %. The pretreated the municipal solid waste can be unwashed or washed using any method known in the art, e.g., washed with water.

Mechanical Pretreatment or Physical Pretreatment: The term “mechanical pretreatment” or “physical pretreatment” refers to any pretreatment that promotes size reduction of particles. For example, such pretreatment can involve various types of grinding or milling (e.g., dry milling, wet milling, or vibratory ball milling).

The municipal solid waste material can be pretreated both physically (mechanically) and chemically. Mechanical or physical pretreatment can be coupled with steaming/steam explosion, hydrothermolysis, dilute or mild acid treatment, high temperature, high pressure treatment, irradiation (e.g., microwave irradiation), or combinations thereof. In one aspect, high pressure means pressure in the range of preferably about 100 to about 400 psi, e.g., about 150 to about 250 psi. In another aspect, high temperature means temperature in the range of about 100 to about 300° C., e.g., about 140 to about 200° C. In a preferred aspect, mechanical or physical pretreatment is performed in a batch-process using a steam gun hydrolyzer system that uses high pressure and high temperature as defined above, e.g., a Sunds Hydrolyzer available from Sunds Defibrator AB, Sweden. The physical and chemical pretreatments can be carried out sequentially or simultaneously, as desired.

Accordingly, in a preferred aspect, the municipal solid waste material is subjected to physical (mechanical) or chemical pretreatment, or any combination thereof, to promote the separation and/or release of cellulose, hemicellulose, and/or lignin.

Biological Pretreatment. The term “biological pretreatment” refers to any biological pretreatment that promotes the separation and/or release of cellulose, hemicellulose, and/or lignin from the municipal solid waste material. Biological pretreatment techniques can involve applying lignin-solubilizing microorganisms and/or enzymes (see, for example, Hsu, T.-A., 1996, Pretreatment of biomass, in Handbook on Bioethanol: Production and Utilization, Wyman, C. E., ed., Taylor & Francis, Washington, D.C., 179-212; Ghosh and Singh, 1993, Adv. Appl. Microbiol. 39: 295-333; McMillan, J. D., 1994, Pretreating lignocellulosic biomass: a review, in Enzymatic Conversion of Biomass for Fuels Production, Himmel, M. E., Baker, J. O., and Overend, R. P., eds., ACS Symposium Series 566, American Chemical Society, Washington, D.C., chapter 15; Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson and Hahn-Hagerdal, 1996, Enz. Microb. Tech. 18: 312-331; and Vallander and Eriksson, 1990, Adv. Biochem. Eng./Biotechnol. 42: 63-95).

In other embodiments MSW can be pretreated both physically (mechanically) and chemically. Mechanical or physical pretreatment can be coupled with steaming/steam explosion, hydrothermolysis, dilute or mild acid treatment, high temperature, high pressure treatment, irradiation (e.g., microwave irradiation), or combinations thereof. In one aspect, high pressure means pressure in the range of preferably about 100 to about 400 psi, e.g., about 150 to about 250 psi. In another aspect, high temperature means temperature in the range of about 100 to about 300° C., e.g., about 140 to about 200° C. In a preferred aspect, mechanical or physical pretreatment is performed in a batch-process using a steam gun hydrolyzer system that uses high pressure and high temperature as defined above, e.g., a Sunds Hydrolyzer available from unds Defibrator AB, Sweden. The physical and chemical pretreatments can be carried out sequentially or simultaneously, as desired.

Solubilization. In the solubilization (hydrolysis) step, the municipal solid waste material, e.g., pretreated, is hydrolyzed to break down cellulose and/or hemicellulose and other substrates to fermentable sugars, such as glucose, cellobiose, xylose, xylulose, arabinose, mannose, galactose, and/or soluble oligosaccharides (also known as saccharification). The hydrolysis is performed enzymatically by one or more enzyme compositions in one or more stages. In the hydrolysis step, the municipal solid waste material, e.g., pretreated, is hydrolyzed to break down proteins and lipids (e.g. triglycerides) found in the waste.

The hydrolysis can be carried out as a batch process or series of batch processes. The hydrolysis can be carried out as a fed batch or continuous process, or series of fed batch or continuous processes, where the municipal solid waste material is fed gradually to, for example, a hydrolysis solution containing an enzyme composition. In an embodiment the hydrolysis a continuous hydrolysis in which a MSW material and a enzymes composition are added at different intervals throughout the hydrolysis and the hydrolysate is removed at different intervals throughout the hydrolysis.

Enzymatic hydrolysis is preferably carried out in a suitable aqueous environment under conditions that can be readily determined by one skilled in the art. In one aspect, hydrolysis is performed under conditions suitable for the activity of the enzymes(s), i.e., optimal for the enzyme(s).

In one aspect, the hydrolysis is performed in the presence of dissolved oxygen at a concentration of at least 0.5% of the saturation level.

In an embodiment of the invention the dissolved oxygen concentration during hydrolysis is in the range of at least 0.5% up to 30% of the saturation level, such as at least 1% up to 25%, at least 1% up to 20%, at least 1% up to 15%, at least 1% up to 10%, at least 1% up to 5%, and at least 1% up to 3%. In a preferred embodiment, the dissolved oxygen concentration is maintained at a concentration of at least 0.5% up to 30% of the saturation level, such as at least 1% up to 25%, at least 1% up to 20%, at least 1% up to 15%, at least 1% up to 10%, at least 1% up to 5%, and at least 1% up to 3% during at least 25%, such as at least 50% or at least 75% of the hydrolysis period. When the enzyme composition comprises an oxidoreductase the dissolved oxygen concentration may be higher up to 70% of the saturation level.

Oxygen is added to the vessel in order to achieve the desired concentration of dissolved oxygen during saccharification. Maintaining the dissolved oxygen level within a desired range can be accomplished by aeration of the vessel, tank or the like by adding compressed air through a diffuser or sparger, or by other known methods of aeration. The aeration rate can be controlled on the basis of feedback from a dissolved oxygen sensor placed in the vessel/tank, or the system can run at a constant rate without feedback control. In the case of a hydrolysis train consisting of a plurality of vessels/tanks connected in series, aeration can be implemented in one or more or all of the vessels/tanks. Oxygen aeration systems are well known in the art. According to the invention any suitable aeration system may be used. Commercial aeration systems are designed by, e.g., Chemineer, Derby, England, and build by, e.g., Paul Mueller Company, MO, USA.

The enzyme compositions can comprise any protein useful in degrading the municipal solid waste material.

In one embodiment, the cellulolytic enzyme compositions comprise (a) an endoglucanase I or variant thereof, (b) an endoglucanase II or variant thereof, (c) a cellobiohydrolase I or variant thereof; (d) a cellobiohydrolase II or variant thereof; (d) a beta-glucosidase or variant thereof; and optionally (e) an AA9 polypeptide having cellulolytic enhancing activity or variant thereof. In another embodiment of the present invention, the cellulolytic enzyme composition comprises hemicelluloytic enzyme composition which is one or more enzymes selected from the group consisting of a xylanase, an acetylxylan esterase, a feruloyl esterase, an arabinofuranosidase, a xylosidase, and a glucuronidase.

In one aspect, the enzyme composition comprises or further comprises one or more (e.g., several) proteins selected from the group consisting of a cellulase, an AA9 polypeptide, a beta-glucanase, a cellulose inducing protein, a hemicellulase, an esterase, an expansin, a ligninolytic enzyme, a lipase, a mannanase, an oxidoreductase, a pectinase, and a swollenin. In another aspect, the cellulase is preferably one or more (e.g., several) enzymes selected from the group consisting of an endoglucanase, a cellobiohydrolase, and a beta-glucosidase. In another aspect, the hemicellulase is preferably one or more (e.g., several) enzymes selected from the group consisting of an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. In another aspect, the oxidoreductase is preferably one or more (e.g., several) enzymes selected from the group consisting of a catalase, a laccase, and a peroxidase.

In another aspect, the enzyme composition comprises one or more (e.g., several) cellulolytic enzymes. In another aspect, the enzyme composition comprises or further comprises one or more (e.g., several) hemicellulolytic enzymes. In another aspect, the enzyme composition comprises one or more (e.g., several) cellulolytic enzymes and one or more (e.g., several) hemicellulolytic enzymes. In another aspect, the enzyme composition comprises one or more (e.g., several) enzymes selected from the group of cellulolytic enzymes and hemicellulolytic enzymes. In another aspect, the enzyme composition comprises an endoglucanase. In another aspect, the enzyme composition comprises a cellobiohydrolase. In another aspect, the enzyme composition comprises a beta-glucosidase. In another aspect, the enzyme composition comprises an AA9 polypeptide. In another aspect, the enzyme composition comprises an endoglucanase and an AA9 polypeptide. In another aspect, the enzyme composition comprises a cellobiohydrolase and an AA9 polypeptide. In another aspect, the enzyme composition comprises a beta-glucosidase and an AA9 polypeptide. In another aspect, the enzyme composition comprises an endoglucanase and a cellobiohydrolase. In another aspect, the enzyme composition comprises an endoglucanase I, an endoglucanase II, or a combination of an endoglucanase I and an endoglucanase II, and a cellobiohydrolase I, a cellobiohydrolase II, or a combination of a cellobiohydrolase I and a cellobiohydrolase II. In another aspect, the enzyme composition comprises an endoglucanase and a beta-glucosidase. In another aspect, the enzyme composition comprises an endoglucanase I, an endoglucanase II, or a combination of an endoglucanase I and an endoglucanase II, and a beta-glucosidase. In another aspect, the enzyme composition comprises a beta-glucosidase and a cellobiohydrolase. In another aspect, the enzyme composition comprises a beta-glucosidase and a cellobiohydrolase I, a cellobiohydrolase II, or a combination of a cellobiohydrolase I and a cellobiohydrolase II. In another aspect, the enzyme composition comprises an endoglucanase, an AA9 polypeptide, and a cellobiohydrolase. In another aspect, the enzyme composition comprises an endoglucanase I, an endoglucanase II, or a combination of an endoglucanase I and an endoglucanase II, an AA9 polypeptide, and a cellobiohydrolase I, a cellobiohydrolase II, or a combination of a cellobiohydrolase I and a cellobiohydrolase II. In another aspect, the enzyme composition comprises an endoglucanase, a beta-glucosidase, and an AA9 polypeptide. In another aspect, the enzyme composition comprises a beta-glucosidase, an AA9 polypeptide, and a cellobiohydrolase. In another aspect, the enzyme composition comprises a beta-glucosidase, an AA9 polypeptide, and a cellobiohydrolase I, a cellobiohydrolase II, or a combination of a cellobiohydrolase I and a cellobiohydrolase II. In another aspect, the enzyme composition comprises an endoglucanase, a beta-glucosidase, and a cellobiohydrolase. In another aspect, the enzyme composition comprises an endoglucanase I, an endoglucanase II, or a combination of an endoglucanase I and an endoglucanase II, a beta-glucosidase, and a cellobiohydrolase I, a cellobiohydrolase II, or a combination of a cellobiohydrolase I and a cellobiohydrolase II. In another aspect, the enzyme composition comprises an endoglucanase, a cellobiohydrolase, a beta-glucosidase, and an AA9 polypeptide. In another aspect, the enzyme composition comprises an endoglucanase I, an endoglucanase II, or a combination of an endoglucanase I and an endoglucanase II, a beta-glucosidase, an AA9 polypeptide, and a cellobiohydrolase I, a cellobiohydrolase II, or a combination of a cellobiohydrolase I and a cellobiohydrolase II.

In another aspect, the enzyme composition comprises an acetylmannan esterase. In another aspect, the enzyme composition comprises an acetylxylan esterase. In another aspect, the enzyme composition comprises an arabinanase (e.g., alpha-L-arabinanase). In another aspect, the enzyme composition comprises an arabinofuranosidase (e.g., alpha-L-arabinofuranosidase). In another aspect, the enzyme composition comprises a coumaric acid esterase. In another aspect, the enzyme composition comprises a feruloyl esterase. In another aspect, the enzyme composition comprises a galactosidase (e.g., alpha-galactosidase and/or beta-galactosidase). In another aspect, the enzyme composition comprises a glucuronidase (e.g., alpha-D-glucuronidase). In another aspect, the enzyme composition comprises a glucuronoyl esterase. In another aspect, the enzyme composition comprises a mannanase. In another aspect, the enzyme composition comprises a mannosidase (e.g., beta-mannosidase). In another aspect, the enzyme composition comprises a xylanase. In an embodiment, the xylanase is a Family 10 xylanase. In another embodiment, the xylanase is a Family 11 xylanase. In another aspect, the enzyme composition comprises a xylosidase (e.g., beta-xylosidase).

In another aspect, the enzyme composition comprises a beta-glucanase. In another aspect, the enzyme composition comprises a cellulose inducing protein. In another aspect, the enzyme composition comprises an esterase. In another aspect, the enzyme composition comprises an expansin. In another aspect, the enzyme composition comprises a ligninolytic enzyme. In an embodiment, the ligninolytic enzyme is a manganese peroxidase. In another embodiment, the ligninolytic enzyme is a lignin peroxidase. In another embodiment, the ligninolytic enzyme is a H₂O₂-producing enzyme. In another aspect, the enzyme composition comprises a lipase. In another aspect, the enzyme composition comprises a pectinase. In another aspect, the enzyme composition comprises a mannanase, In another aspect, the enzyme composition comprises an oxidoreductase. In an embodiment, the oxidoreductase is a catalase. In another embodiment, the oxidoreductase is a laccase. In another embodiment, the oxidoreductase is a peroxidase. In another aspect, the enzyme composition comprises a protease. In another aspect, the enzyme composition comprises a swollenin.

In the processes of the present invention, the enzyme(s) can be added prior to or during hydrolysis, hydrolysis and fermentation, or fermentation.

One or more (e.g., several) components of the enzyme composition may be native proteins, recombinant proteins, or a combination of native proteins and recombinant proteins. For example, one or more (e.g., several) components may be native proteins of a cell, which is used as a host cell to express recombinantly one or more (e.g., several) other components of the enzyme composition. It is understood herein that the recombinant proteins may be heterologous (e.g., foreign) and/or native to the host cell. One or more (e.g., several) components of the enzyme composition may be produced as monocomponents, which are then combined to form the enzyme composition. The enzyme composition may be a combination of multicomponent and monocomponent protein preparations.

The enzymes used in the processes of the present invention may be in any form suitable for use, such as, for example, a fermentation broth formulation or a cell composition, a cell lysate with or without cellular debris, a semi-purified or purified enzyme preparation, or a host cell as a source of the enzymes. The enzyme composition may be a dry powder or granulate, a non-dusting granulate, a liquid, a stabilized liquid, or a stabilized protected enzyme. Liquid enzyme preparations may, for instance, be stabilized by adding stabilizers such as a sugar, a sugar alcohol or another polyol, and/or lactic acid or another organic acid according to established processes.

A fermentation broth formulation or a cell composition comprising enzymes used in the processes of the present invention further comprises additional ingredients used in the fermentation process, such as, for example, cells (including, the host cells containing the gene encoding the polypeptide of the present invention which are used to produce the polypeptide), cell debris, biomass, fermentation media and/or fermentation products. In some embodiments, the composition is a cell-killed whole broth containing organic acid(s), killed cells and/or cell debris, and culture medium.

The term “fermentation broth” refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis (e.g., expression of cellulase and/or glucosidase enzyme(s)). In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

The optimum amounts of the enzymes and polypeptides having enzymatic activity depend on several factors including, but not limited to, the mixture of cellulolytic enzymes and/or hemicellulolytic enzymes, the municipal solid waste material, the concentration of municipal solid waste material, the pretreatment(s) of the municipal solid waste material, temperature, time, pH, and inclusion of a fermenting organism (e.g., for Simultaneous Saccharification and Fermentation).

In one aspect, an effective amount of the enzyme composition to the municipal solid waste material is about 0.5 to about 50 mg, e.g., about 0.5 to about 40 mg, about 0.5 to about 25 mg, about 0.75 to about 20 mg, about 0.75 to about 15 mg, about 0.5 to about 10 mg, or about 2.5 to about 10 mg per g of the municipal solid waste material.

In another embodiment, the enzyme composition comprises a protease which has at least 50%, 55%, 60%, 65%, 70%, 75%, 80%, 85%, 90%, 95% or 100% sequence identity to amino acids 1 to 177 of SEQ ID NO: 1 or a fragment thereof having protease activity, derived from a fungus of the genus Thermoascus, for example the species Thermoascus aurantiacus, such as the strain Thermoascus aurantiacus CGMCC No. 0582.

In another embodiment, the protease has at least 50%, 55%, 60%, 65%, 70%, 75%, 80%, 85%, 90%, 95% or 100% sequence identity to SEQ ID NO: 5, derived from a strain of the bacterium Pyrococcus, such as a strain of Pyrococcus furiosus.

In another embodiment of the present invention, the protease has at least 50%, 55%, 60%, 65%, 70%, 75%, 80%, 85%, 90%, 95% or 100% sequence identity to SEQ ID NO:32 or a fragment thereof having protease activity, derived from a strain of Bacillus clausii.

In another embodiment of the present invention, the protease has at least 50%, 55%, 60%, 65%, 70%, 75%, 80%, 85%, 90%, 95% or 100% sequence identity to SEQ ID NO:33 or a fragment thereof having protease activity, derived from a strain of Bacillus licheniformis.

In another embodiment of the present invention, the protease has at least 50%, 55%, 60%, 65%, 70%, 75%, 80%, 85%, 90%, 95% or 100% sequence identity to SEQ ID NO:36 or a fragment thereof having protease activity, derived from a fungus of the genus Meripilus, for example the species Meripilus giganteus.

In another embodiment of the present invention, the protease has at least 50%, 55%, 60%, 65%, 70%, 75%, 80%, 85%, 90%, 95% or 100% sequence identity to amino acids 199 to 564 of SEQ ID NO:36 or a fragment thereof having protease activity, derived from a fungus of the genus Meripilus, for example the species Meripilus giganteus.

In another embodiment, the protease is selected from a serine protease of family S53, such as from a strain of the genus Meripilus, more particularly Meripilus giganteus

In another embodiment, the protease is present at a ratio between 0.1-2% w/w, preferably, 0.2%, 0.4% or 1% w/w of the total enzyme protein. In a related aspect, the protease is present at a ratio between 0.1-2% w/w, such as0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2% w/w of the total enzyme protein.

In another embodiment, the enzyme composition comprises lipase which has at least 50%, 55%, 60%, 65%, 70%, 75%, 80%, 85%, 90%, 95% or 100% identity with SEQ ID NO: 2, and/or is derived from the genus Thermomyces sp. such as Thermomyces lanuginosus; or, said the lipase is derived from the genus Humicola sp. such as Humicola insolens.

In another embodiment, the lipase is present at a ratio between 0-10% w/w, such as 1, 2, 3, 4, 5, 6, 7, 8, 9, 10% w/w of the total enzyme protein, preferably, 0.1% or 1% w/w of the total enzyme protein.

In another embodiment, the pectinase forms part of a multicomponent enzyme composition comprising pectate lyase, xylanase and cellulase activities such as Pectinex UF or Novozym 81243™. For example, a pectinase derived from Aspergillus aculeatus in wild type.

In another embodiment, the pectinase is present at a ratio between 0-30% w/w, such as 5-20% w/w of the total enzyme protein. In a related aspect, the pectinase is present at a ratio between 0-30% w/w, such as 1, 2, 3, 4, 5, 6, 7, 8, 9, 10, 11, 12, 13, 14, 15, 16, 17, 18, 19, 20, 21, 22, 32, 24, 25, 26, 27, 28, 29 or 30% w/w of the total enzyme protein.

In another embodiment, the mannanase has at least 50%, 55%, 60%, 65%, 70%, 75%, 80%, 85%, 90%, 95% or 100% identity with SEQ ID NO: 3, or homologs thereof, and/or is derived from the genus Bacillus such as Bacillus bogoriensis, or from the genus Talaromyces such as Talaromyces Leycettanus.

In yet another embodiment, the mannanase is present at a ratio between 0-10% w/w, such as 4-5% w/w of the total enzyme protein. In a related aspect, the mannanase is present at a ratio between 0-10% w/w, such as 1, 2, 3, 4, 5, 6, 7, 8, 9, 10% w/w of the total enzyme protein.

In another embodiment, the beta-glucanase has at least 50%, 55%, 60%, 65%, 70%, 75%, 80%, 85%, 90%, 95% or 100% identity with SEQ ID NO: 4 or homologs thereof, and/or is derived from a member of the genus Aspergillus such as Aspergillus aculeatus.

In another embodiment, the beta-glucanase is present at a ratio between 0-30% w/w, such as 10-15% w/w of the total enzyme protein. In one aspect, the beta-glucanase is present at a ratio between 0-30% w/w, such as 1, 2, 3, 4, 5, 6, 7, 8, 9, 10, 11, 12, 13, 14, 15, 16, 17, 18, 19, 20, 21, 22, 32, 24, 25, 26, 27, 28, 29 or 30% w/w of the total enzyme protein.

In another embodiment, the cellulolytic enzyme composition is present at a ratio between 40%-99% w/w, such as between 50%-90% w/w, such as 60%-80% w/w, such as 65-80% of the total enzyme protein.

In yet another related aspect, a cellulolytic enzyme composition is present at a ratio between 40%-99% w/w, such as between 50%-90% w/w, such as 60%-80% w/w, such as 65-80% of the total enzyme protein.

The polypeptides having cellulolytic enzyme activity or hemicellulolytic enzyme activity as well as other proteins/polypeptides useful in the degradation of the municipal solid waste material, e.g., AA9 polypeptides can be derived or obtained from any suitable origin, including, archaeal, bacterial, fungal, yeast, plant, or animal origin. The term “obtained” also means herein that the enzyme may have been produced recombinantly in a host organism employing methods described herein, wherein the recombinantly produced enzyme is either native or foreign to the host organism or has a modified amino acid sequence, e.g., having one or more (e.g., several) amino acids that are deleted, inserted and/or substituted, i.e., a recombinantly produced enzyme that is a mutant and/or a fragment of a native amino acid sequence or an enzyme produced by nucleic acid shuffling processes known in the art. Encompassed within the meaning of a native enzyme are natural variants and within the meaning of a foreign enzyme are variants obtained by, e.g., site-directed mutagenesis or shuffling.

Each polypeptide may be a bacterial polypeptide. For example, each polypeptide may be a Gram-positive bacterial polypeptide having enzyme activity, or a Gram-negative bacterial polypeptide having enzyme activity.

Each polypeptide may also be a fungal polypeptide, e.g., a yeast polypeptide or a filamentous fungal polypeptide.

Chemically modified or protein engineered mutants of polypeptides may also be used.

One or more (e.g., several) components of the enzyme composition may be a recombinant component, i.e., produced by cloning of a DNA sequence encoding the single component and subsequent cell transformed with the DNA sequence and expressed in a host (see, for example, WO 91/17243 and WO 91/17244). The host can be a heterologous host (enzyme is foreign to host), but the host may under certain conditions also be a homologous host (enzyme is native to host). Monocomponent cellulolytic proteins may also be prepared by purifying such a protein from a fermentation broth.

In one aspect, the cellulolytic enzyme composition is a commercial cellulolytic enzyme composition. Examples of commercial cellulolytic enzyme compositions suitable for use in the present invention include, for example, CELLUCLAST®, CELLIC® CTec (Novozymes A/S), CELLIC® CTec2 (Novozymes A/S), CELLIC® CTec3 (Novozymes A/S), CELLUCLAST™ (Novozymes A/S), NOVOZYM™ 188 (Novozymes A/S), SPEZYME™ CP (Genencor Int.), ACCELLERASE™ TRIO (DuPont), FILTRASE® NL (DSM); METHAPLUS® S/L 100 (DSM), ROHAMENT™ 7069 W (Röhm GmbH), or ALTERNAFUEL® CMAX3™ (Dyadic International, Inc.). The cellulolytic enzyme composition is added in an amount effective from about 0.001 to about 5.0 wt. % of solids, e.g., about 0.025 to about 4.0 wt. % of solids or about 0.005 to about 2.0 wt. % of solids.

In another aspect, the cellulolytic enzyme composition comprises a whole Trichoderma reesei cellulase composition.

In another aspect, the cellulolytic enzyme composition is derived from Trichoderma reesei further comprising (a) an AA9 polypeptide having cellulolytic enhancing activity; and (b) a beta-glucosidase;

wherein the AA9 polypeptide having cellulolytic enhancing activity is selected from the group consisting of:

(a) an AA9 polypeptide comprising an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 6, SEQ ID NO: 7, SEQ ID NO: 8, SEQ ID NO: 9, SEQ ID NO: 10, SEQ ID NO: 11, or SEQ ID NO: 12; and/or

(b) an AA9 polypeptide comprising the mature polypeptide of SEQ ID NO: 6, SEQ ID NO: 7, SEQ ID NO: 8, SEQ ID NO: 9, SEQ ID NO: 10, SEQ ID NO: 11, or SEQ ID NO: 12; and

wherein the beta-glucosidase is selected from the group consisting of:

(a) a beta-glucosidase comprising an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 13 or SEQ ID NO: 14; and/or

(b) a beta-glucosidase comprising the mature polypeptide of SEQ ID NO: 13 or SEQ ID NO: 14.

In a preferred embodiment, the cellulolytic enzyme composition is derived from Trichoderma reesei further comprising a beta-glucosidase of SEQ ID NO: 13 or 14 and an AA9 polypeptide of SEQ ID NO: 6, SEQ ID NO: 7, SEQ ID NO: 8, SEQ ID NO: 9, SEQ ID NO: 10, SEQ ID NO: 11, or SEQ ID NO: 12.

In another preferred embodiment, the cellulolytic enzyme composition is derived from Trichoderma reesei further comprising a beta-glucosidase of SEQ ID NO: 13 or 14 and an AA9 polypeptide of SEQ ID NO: 6.

In another aspect, the cellulolytic enzyme composition is derived from Trichoderma reesei further comprising (i) a cellobiohydrolase I; (ii) a cellobiohydrolase II; (iii) a beta-glucosidase or variant thereof; and (iv) an AA9 polypeptide having cellulolytic enhancing activity; or homologs thereof, wherein the cellobiohydrolase I or homolog thereof is selected from the group consisting of:

(i) a cellobiohydrolase I comprising or consisting of the mature polypeptide of SEQ ID NO: 15; and/or

(ii) a cellobiohydrolase I comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 15;

wherein the cellobiohydrolase II or homolog thereof is selected from the group consisting of:

(i) a cellobiohydrolase II comprising or consisting of the mature polypeptide of SEQ ID NO: 16; and/or

(ii) a cellobiohydrolase II comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 16;

wherein the beta-glucosidase or homolog thereof is selected from the group consisting of:

(i) a beta-glucosidase comprising or consisting of the mature polypeptide of SEQ ID NO: 17;

(ii) a beta-glucosidase comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 17; and/or

(v) a beta-glucosidase variant comprising a substitution at one or more positions corresponding to positions 100, 283, 456, and 512 of the mature polypeptide of SEQ ID NO: 17, wherein the variant has beta-glucosidase activity; and

wherein the AA9 polypeptide having cellulolytic enhancing activity or homolog thereof is selected from the group consisting of:

(i) an AA9 polypeptide having cellulolytic enhancing activity comprising or consisting of the mature polypeptide of SEQ ID NO: 18; and/or

(ii) an AA9 polypeptide having cellulolytic enhancing activity comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 18.

In an embodiment, the beta-glucosidase variant comprises one or more (several) substitutions selected from the group consisting of G142S, Q183R, H266Q, and D703G of SEQ ID NO: 17.

In another embodiment, the cellulolytic enzyme composition further comprises one or more enzymes selected from the group consisting of: (i) a xylanase or homolog thereof, (ii) a beta-xylosidase or homolog thereof; or (iii) a combination of (i) and (ii);

wherein the xylanase or homolog thereof is selected from the group consisting of:

(i) a xylanase comprising or consisting of the mature polypeptide of SEQ ID NO: 19, SEQ ID NO: 20, or SEQ ID NO: 21; and/or

(ii) a xylanase comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 19, SEQ ID NO: 20, or SEQ ID NO: 21; and

wherein the beta-xylosidase or homolog thereof is selected from the group consisting of:

(i) a beta-xylosidase comprising or consisting of the mature polypeptide of SEQ ID NO: 22; and/or

(ii) a beta-xylosidase comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 22.

In a preferred embodiment, the cellulolytic enzyme composition is derived from Trichoderma reesei further comprising the cellobiohyolase I of the mature polypeptide of SEQ ID NO: 15, the cellobiohydrolase II of the mature polypeptide of SEQ ID NO: 16, the beta-glucosidase of the mature polypeptide of SEQ ID NO: 17, the AA9 polypeptide of the mature polypeptide of SEQ ID NO: 18, and optionally the xylanase of the mature polypeptide of SEQ ID NO: 21 and/or the beta-xylosidase of the mature polypeptide of SEQ ID NO: 22.

In a preferred embodiment, the cellulolytic enzyme composition is derived from Trichoderma reesei further comprising the cellobiohyolase I of the mature polypeptide of SEQ ID NO: 15, the cellobiohydrolase II of the mature polypeptide of SEQ ID NO: 16, the beta-glucosidase of the mature polypeptide of SEQ ID NO: 17, the AA9 polypeptide of the mature polypeptide of SEQ ID NO: 18, and optionally the xylanase of the mature polypeptide of SEQ ID NO: 21 and/or the beta-xylosidase of the mature polypeptide of SEQ ID NO: 22 and the mannanase of the mature polypeptide of SEQ ID NO: 3.

In another embodiment, the cellulolytic enzyme composition further comprises one or more enzymes selected from the group consisting of: (i) a GH10 xylanase or homolog thereof; and (ii) a beta-xylosidase or homolog thereof. or (iii) a combination of (i) and (ii); wherein the GH10 xylanase is selected from the group consisting of: (i) a xylanase comprising or consisting of the mature polypeptide of SEQ ID NO: 23; and/or (ii) a xylanase comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 23.

wherein the beta-xylosidase is selected from the group consisting of: (i) a beta-xylosidase comprising or consisting of the mature polypeptide of SEQ ID NO: 24; and/or (ii) a beta-xylosidase comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 24.

In a preferred embodiment, the cellulolytic enzyme composition is derived from Trichoderma reesei further comprising the cellobiohydrolase I of the mature polypeptide of SEQ ID NO: 16, the cellobiohydrolase II of the mature polypeptide of SEQ ID NO: 16, the beta-glucosidase of the mature polypeptide of SEQ ID NO: 17, the AA9 polypeptide of the mature polypeptide of SEQ ID NO: 18, and optionally the xylanase of the mature polypeptide of SEQ ID NO: 23 and/or the beta-xylosidase of the mature polypeptide of SEQ ID NO: 24.

In a preferred embodiment, the cellulolytic enzyme composition is derived from Trichoderma reesei further comprising the cellobiohydolase I of the mature polypeptide of SEQ ID NO: 15, the cellobiohydrolase II of the mature polypeptide of SEQ ID NO: 16, the beta-glucosidase of the mature polypeptide of SEQ ID NO: 17, the AA9 polypeptide of the mature polypeptide of SEQ ID NO: 18, and optionally the xylanase of the mature polypeptide of SEQ ID NO: 23 and/or the beta-xylosidase of the mature polypeptide of SEQ ID NO: 24 and the mannanase of the mature polypeptide of SEQ ID NO: 3.

In another aspect, the cellulolytic enzyme composition comprises (A) (i) a cellobiohydrolase I, (ii) a cellobiohydrolase II, and (iii) at least one enzyme selected from the group consisting of a beta-glucosidase or a variant thereof, an AA9 polypeptide having cellulolytic enhancing activity, a GH10 xylanase, and a beta-xylosidase; (B) (i) a GH10 xylanase and (ii) a beta-xylosidase; or (C) (i) a cellobiohydrolase I, (ii) a cellobiohydrolase II, (iii) a GH10 xylanase, and (iv) a beta-xylosidase;

wherein the cellobiohydrolase I is selected from the group consisting of: (i) a cellobiohydrolase I comprising or consisting of the mature polypeptide of SEQ ID NO: 25; and/or (ii) a cellobiohydrolase I comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 25;

wherein the cellobiohydrolase II is selected from the group consisting of: (i) a cellobiohydrolase II comprising or consisting of the mature polypeptide of SEQ ID NO: 26; and/or (ii) a cellobiohydrolase II comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 26;

wherein the beta-glucosidase is selected from the group consisting of: (i) a beta-glucosidase comprising or consisting of the mature polypeptide of SEQ ID NO: 17; and/or (ii) a beta-glucosidase comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 17;

wherein the AA9 polypeptide is selected from the group consisting of: an AA9 polypeptide comprising or consisting of the mature polypeptide of SEQ ID NO: 18; and/or (ii) an AA9 polypeptide comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 18;

wherein the xylanase is selected from the group consisting of: (i) a xylanase comprising or consisting of the mature polypeptide of SEQ ID NO: 27 or the mature polypeptide of SEQ ID NO: 27; and/or (ii) a xylanase comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 28 or the mature polypeptide of SEQ ID NO: 28; and

wherein the beta-xylosidase is selected from the group consisting of: (i) a beta-xylosidase comprising or consisting of the mature polypeptide of SEQ ID NO: 24; and/or (ii) a beta-xylosidase comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 24.

In another embodiment, the enzyme compositions described above further comprise an endoglucanase I, an endoglucanase II, or an endoglucanase I and an endoglucanase II, wherein the endoglucanase I is selected from the group consisting of: (i) an endoglucanase I comprising or consisting of the mature polypeptide of SEQ ID NO: 29; and/or (ii) an endoglucanase I comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 29; and

wherein the endoglucanase II is selected from the group consisting of: (i) an endoglucanase II comprising or consisting of the mature polypeptide of SEQ ID NO: 30; and/or (ii) an endoglucanase II comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 30; or wherein the endoglucanase II is selected from the group consisting of: (i) an endoglucanase II comprising or consisting of the mature polypeptide of SEQ ID NO: 31; and/or (ii) an endoglucanase II comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 31.

In a preferred embodiment, the cellulolytic enzyme composition is derived from Trichoderma reesei further comprising the cellobiohydrolase I of the mature polypeptide of SEQ ID NO: 25, the cellobiohydrolase II of the mature polypeptide of SEQ ID NO: 26, the beta-glucosidase of the mature polypeptide of SEQ ID NO: 17, the AA9 polypeptide of the mature polypeptide of SEQ ID NO: 18, and optionally the xylanase of the mature polypeptide of SEQ ID NO: 27 or SEQ ID NO: 28 and/or the beta-xylosidase of the mature polypeptide of SEQ ID NO: 24.

The present invention is further described by the following numbered paragraphs.

Paragraph [1]. A process for solubilizing a municipal solid waste, comprising treating the municipal solid waste with an effective amount of (i) a cellulolytic enzyme composition, and (ii) a protease selected from the group consisting of: (a) a protease having at least 50%, 55%, 60%, 65%, 70%, 75%, 80%, 85%, 90%, 95% or 100% identity to amino acids 1 to 177 of SEQ ID NO: 1 or a fragment thereof having protease activity; (b) a protease having at least 50%, 55%, 60%, 65%, 70%, 75%, 80%, 85%, 90%, 95% or 100% identity to SEQ ID NO: 5; (c) a protease having at least 50%, 55%, 60%, 65%, 70%, 75%, 80%, 85%, 90%, 95% or 100% identity to SEQ ID NO: 32 or a variant thereof; (d) a protease having at least 50%, 55%, 60%, 65%, 70%, 75%, 80%, 85%, 90%, 95% or 100% identity to amino acids of SEQ ID NO: 33; and (e) a protease having at least 50%, 55%, 60%, 65%, 70%, 75%, 80%, 85%, 90%, 95% or 100% identity to amino acids 199 to 564 of SEQ ID NO: 36 to produce a solubilized municipal solid waste and optionally recovering the solubilized municipal solid waste.

Paragraph [2]. The process of paragraph 1, further comprising treating the municipal solid waste with one or more enzymes selected from a cellulase, an AA9 polypeptide, a beta-glucanase, a cellulose inducing protein, a hemicellulase, an esterase, an expansin, a ligninolytic enzyme, a lipase, a mannanase, an oxidoreductase, a pectinase, and a swollenin.

Paragraph [3]. The process of paragraph 2, wherein the cellulase is one or more enzymes selected from the group consisting of an endoglucanase, a cellobiohydrolase, and a beta-glucosidase.

Paragraph [4]. The process of paragraph 2, wherein the hemicellulase is one or more enzymes selected from the group consisting of a xylanase, an acetylxylan esterase, a feruloyl esterase, an arabinofuranosidase, a xylosidase, and a glucuronidase.

Paragraph [5]. The process of any one of paragraphs 1-4, further comprising treating the municipal solid waste with one or more enzymes selected from a lipase, a mannanase, a pectinase, and a beta-glucanase.

Paragraph [6]. The process of paragraph 1, wherein the cellulolytic enzyme composition comprises (a) an endoglucanase I, (b) an endoglucanase II, (c) a cellobiohydrolase I or variant thereof; (d) cellobiohydrolase II or variant thereof; (e) beta-glucosidase or variant thereof; (f) an AA9 polypeptide having cellulolytic enhancing activity; and optionally (g) a xylanase and/or (h) a beta-xylosidase.

Paragraph [7]. The process of paragraph 1, wherein the cellulolytic enzyme composition derived from Trichoderma reesei further comprising the cellobiohydrolase I of SEQ ID NO: 15, the cellobiohydrolase II of SEQ ID NO: 16, the beta-glucosidase of SEQ ID NO: 17, the AA9 (GH61) polypeptide having cellulolytic enhancing activity of SEQ ID NO: 18, the xylanase of SEQ ID NO: 21, and the beta-xylosidase of SEQ ID NO: 22.

Paragraph [8]. The process of any one of paragraphs 1-7, wherein, said protease is derived from a fungus of the genus Thermoascus, for example the species Thermoascus aurantiacus, such as the strain Thermoascus aurantiacus CGMCC No. 0582, or said protease is derived from a strain of Pyrococcus furiosus, Bacillus clausii, Bacillus licheniformis, or Meripilus giganteus.

Paragraph [9]. The process of paragraph 5, wherein the lipase has at least 50%, 55%, 60%, 65%, 70%, 75%, 80%, 85%, 90%, 95% or 100% identity with SEQ ID NO: 2, and/or is derived from the genus Thermomyces sp. such as Thermomyces lanuginosus; or, said the lipase is derived from the genus Humicola sp. such as Humicola insolens.

Paragraph [10]. The process of paragraph 5, wherein the mannanase is an endo-mannosidase with at least 50%, 55%, 60%, 65%, 70%, 75%, 80%, 85%, 90%, 95% or 100% identity with SEQ ID NO: 3 or homologs thereof, and/or is derived from the genus Bacillus such as Bacillus bogoriensis, or from the genus Talaromyces such as Talaromyces Leycettanus.

Paragraph [11]. The process of paragraph 5, wherein the beta-glucanase has at least 50%, 55%, 60%, 65%, 70%, 75%, 80%, 85%, 90%, 95% or 100% identity with SEQ ID NO: 4 or homologs thereof, and/or is derived from a member of the genus Aspergillus such as Aspergillus aculeatus.

Paragraph [12]. The process of any one of paragraphs 1-11, wherein the protease is present at a ratio between 0.1 -2% w/w, preferably, 0.2%, 0.4% or 1% w/w of the total enzyme protein.

Paragraph [13]. The process of paragraph 5 or 9, wherein, the lipase is present at a ratio between 0-10% w/w, preferably, 0.1% or 1% w/w of the total enzyme protein.

Paragraph [14]. The process of paragraph 5 or 10, wherein the mannanase is present at a ratio between 0-10% w/w, such as 4-5% w/w of the total enzyme protein.

Paragraph [15]. The process of paragraph 5, wherein the pectinase is present at a ratio between 0-30% w/w, such as 5-20% w/w of the total enzyme protein.

Paragraph [16]. The process of paragraph 5 or 11, wherein the beta-glucanase is present at a ratio between 0-30% w/w, such as 10-15% w/w of the total enzyme protein.

Paragraph [17]. The process of any one of paragraphs 1-16, wherein the cellulolytic enzyme composition is present at a ratio between 40%-99% w/w, such as between 50%-90% w/w, such as 60%-80% w/w, such as 65-80% of the total enzyme protein.

Paragraph [18]. A process for solubilizing a municipal solid waste, comprising treating the municipal solid waste with an effective amount of (i) a cellulolytic enzyme composition, (ii) a protease selected from the group consisting of: (a) a protease having at least 50%, 55%, 60%, 65%, 70%, 75%, 80%, 85%, 90%, 95% or 100% identity to amino acids 1 to 177 of SEQ ID NO: 1 or a fragment thereof having protease activity; (b) a protease having at least 50%, 55%, 60%, 65%, 70%, 75%, 80%, 85%, 90%, 95% or 100% identity to SEQ ID NO: 5; (c) a protease having at least 50%, 55%, 60%, 65%, 70%, 75%, 80%, 85%, 90%, 95% or 100% identity to SEQ ID NO: 32 or a variant thereof; (d) a protease having at least 50%, 55%, 60%, 65%, 70%, 75%, 80%, 85%, 90%, 95% or 100% identity to amino acids of SEQ ID NO: 33; and (e) a protease having at least 50%, 55%, 60%, 65%, 70%, 75%, 80%, 85%, 90%, 95% or 100% identity to amino acids 199 to 564 of SEQ ID NO: 36; and (iii) a mannanase to produce a solubilized municipal solid waste and optionally recovering the solubilized municipal solid waste.

Paragraph [19]. A process for solubilizing a municipal solid waste, comprising treating the municipal solid waste with an effective amount of (i) a cellulolytic enzyme composition, and (ii) a protease selected from the group consisting of: (a) a protease having at least 50%, 55%, 60%, 65%, 70%, 75%, 80%, 85%, 90%, 95% or 100% identity to amino acids 1 to 177 of SEQ ID NO: 1 or a fragment thereof having protease activity; (b) a protease having at least 50%, 55%, 60%, 65%, 70%, 75%, 80%, 85%, 90%, 95% or 100% identity to SEQ ID NO: 5; (c) a protease having at least 50%, 55%, 60%, 65%, 70%, 75%, 80%, 85%, 90%, 95% or 100% identity to SEQ ID NO: 32 or a variant thereof; (d) a protease having at least 50%, 55%, 60%, 65%, 70%, 75%, 80%, 85%, 90%, 95% or 100% identity to amino acids of SEQ ID NO: 33; and (e) a protease having at least 50%, 55%, 60%, 65%, 70%, 75%, 80%, 85%, 90%, 95% or 100% identity to SEQ ID NO: 36 or a fragment thereof having protease activity to produce a solubilized municipal solid waste and optionally recovering the solubilized municipal solid waste.

Paragraph [20]. A process for solubilizing a municipal solid waste, comprising treating the municipal solid waste with an effective amount of (i) a cellulolytic enzyme composition, (ii) a protease selected from the group consisting of: (a) a protease having at least 50%, 55%, 60%, 65%, 70%, 75%, 80%, 85%, 90%, 95% or 100% identity to amino acids 1 to 177 of SEQ ID NO: 1 or a fragment thereof having protease activity; (b) a protease having at least 50%, 55%, 60%, 65%, 70%, 75%, 80%, 85%, 90%, 95% or 100% identity to SEQ ID NO: 5; (c) a protease having at least 50%, 55%, 60%, 65%, 70%, 75%, 80%, 85%, 90%, 95% or 100% identity to SEQ ID NO: 32 or a variant thereof; (d) a protease having at least 50%, 55%, 60%, 65%, 70%, 75%, 80%, 85%, 90%, 95% or 100% identity to amino acids of SEQ ID NO: 33; and (e) a protease having at least 50%, 55%, 60%, 65%, 70%, 75%, 80%, 85%, 90%, 95% or 100% identity to SEQ ID NO: 36 or a fragment thereof having protease activity; and (iii) a mannanase to produce a solubilized municipal solid waste and optionally recovering the solubilized municipal solid waste.

Paragraph [21]. An enzyme composition for solubilizing a municipal solid waste, said composition comprising: (i) a cellulolytic enzyme composition, and (ii) a protease selected from the group consisting of: (a) a protease having at least 50%, 55%, 60%, 65%, 70%, 75%, 80%, 85%, 90%, 95% or 100% identity to amino acids 1 to 177 of SEQ ID NO: 1 or a fragment thereof having protease activity; (b) a protease having at least 50%, 55%, 60%, 65%, 70%, 75%, 80%, 85%, 90%, 95% or 100% identity to SEQ ID NO: 5; (c) a protease having at least 50%, 55%, 60%, 65%, 70%, 75%, 80%, 85%, 90%, 95% or 100% identity to SEQ ID NO: 32 or a variant thereof; (d) a protease having at least 50%, 55%, 60%, 65%, 70%, 75%, 80%, 85%, 90%, 95% or 100% identity to SEQ ID NO: 33; and (e) a protease having at least 50%, 55%, 60%, 65%, 70%, 75%, 80%, 85%, 90%, 95% or 100% identity to amino acids 199 to 564 of SEQ ID NO: 36.

Paragraph [22]. An enzyme composition for solubilizing a municipal solid waste, said composition comprising: (i) a cellulolytic enzyme composition, and (ii) a protease selected from the group consisting of: (a) a protease having at least 50%, 55%, 60%, 65%, 70%, 75%, 80%, 85%, 90%, 95% or 100% identity to amino acids 1 to 177 of SEQ ID NO: 1 or a fragment thereof having protease activity; (b) a protease having at least 50%, 55%, 60%, 65%, 70%, 75%, 80%, 85%, 90%, 95% or 100% identity to SEQ ID NO: 5; (c) a protease having at least 50%, 55%, 60%, 65%, 70%, 75%, 80%, 85%, 90%, 95% or 100% identity to SEQ ID NO: 32 or a variant thereof; (d) a protease having at least 50%, 55%, 60%, 65%, 70%, 75%, 80%, 85%, 90%, 95% or 100% identity to SEQ ID NO: 33; and (e) a protease having at least 50%, 55%, 60%, 65%, 70%, 75%, 80%, 85%, 90%, 95% or 100% identity to SEQ ID NO: 36 or a fragment thereof having protease activity.

Paragraph [23]. An enzyme composition for solubilizing a municipal solid waste, said composition comprising: (i) a cellulolytic enzyme composition, (ii) a protease selected from the group consisting of: (a) a protease having at least 50%, 55%, 60%, 65%, 70%, 75%, 80%, 85%, 90%, 95% or 100% identity to amino acids 1 to 177 of SEQ ID NO: 1 or a fragment thereof having protease activity; (b) a protease having at least 50%, 55%, 60%, 65%, 70%, 75%, 80%, 85%, 90%, 95% or 100% identity to SEQ ID NO: 5; (c) a protease having at least 50%, 55%, 60%, 65%, 70%, 75%, 80%, 85%, 90%, 95% or 100% identity to SEQ ID NO: 32 or a variant thereof; (d) a protease having at least 50%, 55%, 60%, 65%, 70%, 75%, 80%, 85%, 90%, 95% or 100% identity to SEQ ID NO: 33; and (e) a protease having at least 50%, 55%, 60%, 65%, 70%, 75%, 80%, 85%, 90%, 95% or 100% identity to amino acids 199 to 564 of SEQ ID NO: 36; and (iii) a mannanase.

Paragraph [24]. An enzyme composition for solubilizing a municipal solid waste, said composition comprising: (i) a cellulolytic enzyme composition, (ii) a protease selected from the group consisting of: (a) a protease having at least 50%, 55%, 60%, 65%, 70%, 75%, 80%, 85%, 90%, 95% or 100% identity to amino acids 1 to 177 of SEQ ID NO: 1 or a fragment thereof having protease activity; (b) a protease having at least 50%, 55%, 60%, 65%, 70%, 75%, 80%, 85%, 90%, 95% or 100% identity to SEQ ID NO: 5; (c) a protease having at least 50%, 55%, 60%, 65%, 70%, 75%, 80%, 85%, 90%, 95% or 100% identity to SEQ ID NO: 32 or a variant thereof; (d) a protease having at least 50%, 55%, 60%, 65%, 70%, 75%, 80%, 85%, 90%, 95% or 100% identity to SEQ ID NO: 33; and (e) a protease having at least 50%, 55%, 60%, 65%, 70%, 75%, 80%, 85%, 90%, 95% or 100% identity to SEQ ID NO: 36; and (iii) a mannanase.

Paragraph [25]. The process of any one of paragraphs 1-20 wherein the municipal solid waste is unsorted prior to solubilization.

Examples of bacterial endoglucanases that can be used in the processes of the present invention, include, but are not limited to, Acidothermus cellulolyticus endoglucanase (WO 91/05039; WO 93/15186; U.S. Pat. No. 5,275,944; WO 96/02551; U.S. Pat. No. 5,536,655; WO 00/70031; WO 05/093050), Erwinia carotovara endoglucanase (Saarilahti et al., 1990, Gene 90: 9-14), Thermobifida fusca endoglucanase III (WO 05/093050), and Thermobifida fusca endoglucanase V (WO 05/093050).

Examples of fungal endoglucanases that can be used in the present invention, include, but are not limited to, Trichoderma reesei endoglucanase I (Penttila et al., 1986, Gene 45: 253-263, Trichoderma reesei Cel7B endoglucanase I (GenBank:M15665), Trichoderma reesei endoglucanase II (Saloheimo et al., 1988, Gene 63:11-22), Trichoderma reesei Cel5A endoglucanase II (GenBank:M19373), Trichoderma reesei endoglucanase III (Okada et al., 1988, Appl. Environ. Microbiol. 64: 555-563, GenBank:AB003694), Trichoderma reesei endoglucanase V (Saloheimo et al., 1994, Molecular Microbiology 13: 219-228, GenBank:Z33381), Aspergillus aculeatus endoglucanase (Ooi et al., 1990, Nucleic Acids Research 18: 5884), Aspergillus kawachii endoglucanase (Sakamoto et al., 1995, Current Genetics 27: 435-439), Fusarium oxysporum endoglucanase (GenBank:L29381), Humicola grisea var. thermoidea endoglucanase (GenBank:AB003107), Melanocarpus albomyces endoglucanase (GenBank:MAL515703), Neurospora crassa endoglucanase (Gen Bank:XM_324477), Humicola insolens endoglucanase V, Myceliophthora thermophila CBS 117.65 endoglucanase, Thermoascus aurantiacus endoglucanase I (Gen Bank:AF487830), Trichoderma reesei strain No. VTT-D-80133 endoglucanase (GenBank:M15665), and Penicillium pinophilum endoglucanase (WO 2012/062220).

Examples of cellobiohydrolases useful in the present invention include, but are not limited to, Aspergillus aculeatus cellobiohydrolase II (WO 2011/059740), Aspergillus fumigatus cellobiohydrolase I (WO 2013/028928), Aspergillus fumigatus cellobiohydrolase II (WO 2013/028928), Chaetomium thermophilum cellobiohydrolase I, Chaetomium thermophilum cellobiohydrolase II, Humicola insolens cellobiohydrolase I, Myceliophthora thermophila cellobiohydrolase II (WO 2009/042871), Penicillium occitanis cellobiohydrolase I (Gen Bank:AY690482), Talaromyces emersonii cellobiohydrolase I (Gen Bank:AF439936), Thielavia hyrcanie cellobiohydrolase II (WO 2010/141325), Thielavia terrestris cellobiohydrolase II (CEL6A, WO 2006/074435), Trichoderma reesei cellobiohydrolase I, Trichoderma reesei cellobiohydrolase II, and Trichophaea saccata cellobiohydrolase II (WO 2010/057086).

Examples of beta-glucosidases useful in the present invention include, but are not limited to, beta-glucosidases from Aspergillus aculeatus (Kawaguchi et al., 1996, Gene 173: 287-288), Aspergillus fumigatus (WO 2005/047499), Aspergillus niger (Dan et al., 2000, J. Biol. Chem. 275: 4973-4980), Aspergillus oryzae (WO 02/095014), Penicillium brasilianum IBT 20888 (WO 2007/019442 and WO 2010/088387), Thielavia terrestris (WO 2011/035029), and Trichophaea saccata (WO 2007/019442).

Other useful endoglucanases, cellobiohydrolases, and beta-glucosidases are disclosed in numerous Glycosyl Hydrolase families using the classification according to Henrissat, 1991, Biochem. J. 280: 309-316, and Henrissat and Bairoch, 1996, Biochem. J. 316: 695-696.

In the processes of the present invention, any AA9 polypeptide can be used as a component of the enzyme composition.

Examples of AA9 polypeptides useful in the processes of the present invention include, but are not limited to, AA9 polypeptides from Thielavia terrestris (WO 2005/074647, WO 2008/148131, and WO 2011/035027), Thermoascus aurantiacus (WO 2005/074656 and WO 2010/065830), Trichoderma reesei (WO 2007/089290 and WO 2012/149344), Myceliophthora thermophila (WO 2009/085935, WO 2009/085859, WO 2009/085864, WO 2009/085868, and WO 2009/033071), Aspergillus fumigatus (WO 2010/138754), Penicillium pinophilum (WO 2011/005867), Thermoascus sp. (WO 2011/039319), Penicillium sp. (emersoni0 (WO 2011/041397 and WO 2012/000892), Thermoascus crustaceous (WO 2011/041504), Aspergillus aculeatus (WO 2012/125925), Thermomyces lanuginosus (WO 2012/113340, WO 2012/129699, WO 2012/130964, and WO 2012/129699), Aurantiporus alborubescens (WO 2012/122477), Trichophaea saccata (WO 2012/122477), Penicillium thomii (WO 2012/122477), Talaromyces stipitatus (WO 2012/135659), Humicola insolens (WO 2012/146171), Malbranchea cinnamomea (WO 2012/101206), Talaromyces leycettanus (WO 2012/101206), and Chaetomium thermophilum (WO 2012/101206), and Talaromyces thermophilus (WO 2012/129697 and WO 2012/130950).

In one aspect, the AA9 polypeptide is used in the presence of a soluble activating divalent metal cation according to WO 2008/151043, e.g., manganese or copper.

In another aspect, the AA9 polypeptide is used in the presence of a dioxy compound, a bicylic compound, a heterocyclic compound, a nitrogen-containing compound, a quinone compound, a sulfur-containing compound, or a liquor obtained from a pretreated municipal solid waste material such as pretreated corn stover (WO 2012/021394, WO 2012/021395, WO 2012/021396, WO 2012/021399, WO 2012/021400, WO 2012/021401, WO 2012/021408, and WO 2012/021410).

The term “liquor” means the solution phase, either aqueous, organic, or a combination thereof, arising from treatment of a lignocellulose and/or hemicellulose material in a slurry, or monosaccharides thereof, e.g., xylose, arabinose, mannose, etc., under conditions as described in WO 2012/021401, and the soluble contents thereof. A liquor for cellulolytic enhancement of an AA9 polypeptide can be produced by treating a lignocellulose or hemicellulose material (or feedstock) by applying heat and/or pressure, optionally in the presence of a catalyst, e.g., acid, optionally in the presence of an organic solvent, and optionally in combination with physical disruption of the material, and then separating the solution from the residual solids. Such conditions determine the degree of cellulolytic enhancement obtainable through the combination of liquor and an AA9 polypeptide during hydrolysis of a cellulosic substrate by a cellulolytic enzyme preparation. The liquor can be separated from the treated material using a method standard in the art, such as filtration, sedimentation, or centrifugation.

In one aspect, an effective amount of the liquor to cellulose is about 10⁻⁶ to about 10 g per g of cellulose, e.g., about 10⁻⁶ to about 7.5 g, about 10⁻⁶ to about 5 g, about 10⁻⁶ to about 2.5 g, about 10⁻⁶ to about 1 g, about 10⁻⁵ to about 1 g, about 10⁻⁵ to about 10⁻¹ g, about 10⁻⁴ to about 10⁻¹ g, about 10⁻³ to about 10⁻¹ g, or about 10⁻³ to about 10⁻² g per g of cellulose.

In one aspect, the one or more (e.g., several) hemicellulolytic enzymes comprise a commercial hemicellulolytic enzyme preparation. Examples of commercial hemicellulolytic enzyme preparations suitable for use in the present invention include, for example, SHEARZYME™ (Novozymes A/S), CELLIC® HTec (Novozymes A/S), CELLIC® HTec2 (Novozymes A/S), CELLIC® HTec3 (Novozymes A/S), VISCOZYME® (Novozymes A/S), ULTRAFLO® (Novozymes A/S), PULPZYME® HC (Novozymes A/S), MULTIFECT® Xylanase (Genencor), ACCELLERASE® XY (Genencor), ACCELLERASE® XC (Genencor), ECOPULP® TX-200A (AB Enzymes), HSP 6000 Xylanase (DSM), DEPOL™ 333P (Biocatalysts Limit, Wales, UK), DEPOL™ 740L. (Biocatalysts Limit, Wales, UK), and DEPOL™ 762P (Biocatalysts Limit, Wales, UK), ALTERNA FUEL 100P (Dyadic), and ALTERNA FUEL 200P (Dyadic).

Examples of xylanases useful in the processes of the present invention include, but are not limited to, xylanases from Aspergillus aculeatus (GeneSeqP:AAR63790; WO 94/21785), Aspergillus fumigatus (WO 2006/078256), Penicillium pinophilum (WO 2011/041405), Penicillium sp. (WO 2010/126772), Thermomyces lanuginosus (GeneSeqP:BAA22485), Talaromyces thermophilus (GeneSeqP:BAA22834), Thielavia terrestris NRRL 8126 (WO 2009/079210), and Trichophaea saccata (WO 2011/057083).

Examples of beta-xylosidases useful in the processes of the present invention include, but are not limited to, beta-xylosidases from Neurospora crassa (SwissProt:Q7SOW4), Trichoderma reesei (UniProtKB/TrEMBL:Q92458), Talaromyces emersonii (SwissProt:Q8X212), and Talaromyces thermophilus (GeneSeqP:BAA22816).

Examples of acetylxylan esterases useful in the processes of the present invention include, but are not limited to, acetylxylan esterases from Aspergillus aculeatus (WO 2010/108918), Chaetomium globosum (UniProt:Q2GWX4), Chaetomium gracile (GeneSeqP:AAB82124), Humicola insolens DSM 1800 (WO 2009/073709), Hypocrea jecorina (WO 2005/001036), Myceliophtera thermophila (WO 2010/014880), Neurospora crassa (UniProt:q7s259), Phaeosphaeria nodorum (UniProt:Q0UHJ1), and Thielavia terrestris NRRL 8126 (WO 2009/042846).

Examples of feruloyl esterases (ferulic acid esterases) useful in the processes of the present invention include, but are not limited to, feruloyl esterases form Humicola insolens DSM 1800 (WO 2009/076122), Neosartorya fischeri (UniProt:A1D9T4), Neurospora crassa (UniProt:Q9HGR3), Penicillium aurantiogriseum (WO 2009/127729), and Thielavia terrestris (WO 2010/053838 and WO 2010/065448).

Examples of arabinofuranosidases useful in the processes of the present invention include, but are not limited to, arabinofuranosidases from Aspergillus niger (GeneSeqP:AAR94170), Humicola insolens DSM 1800 (WO 2006/114094 and WO 2009/073383), and M. giganteus (WO 2006/114094).

Examples of alpha-glucuronidases useful in the processes of the present invention include, but are not limited to, alpha-glucuronidases from Aspergillus clavatus (UniProt:alcc12), Aspergillus fumigatus (Swiss Prot:Q4WW45), Aspergillus niger (UniProt:Q96WX9), Aspergillus terreus (SwissProt:Q0CJP9), Humicola insolens (WO 2010/014706), Penicillium aurantiogriseum (WO 2009/068565), Talaromyces emersonii (UniProt:Q8X211), and Trichoderma reesei (UniProt:Q99024).

Examples of oxidoreductases useful in the processes of the present invention include, but are not limited to, Aspergillus lentilus catalase, Aspergillus fumigatus catalase, Aspergillus niger catalase, Aspergillus oryzae catalase, Humicola insolens catalase, Neurospora crassa catalase, Penicillium emersonii catalase, Scytalidium thermophilum catalase, Talaromyces stipitatus catalase, Thermoascus aurantiacus catalase, Coprinus cinereus laccase, Myceliophthora thermophila laccase, Polyporus pinsitus laccase, Pycnoporus cinnabarinus laccase, Rhizoctonia solani laccase, Streptomyces coelicolor laccase, Coprinus cinereus peroxidase, Soy peroxidase, Royal palm peroxidase.

The polypeptides having enzyme activity used in the processes of the present invention may be produced by fermentation of the above-noted microbial strains on a nutrient medium containing suitable carbon and nitrogen sources and inorganic salts, using procedures known in the art (see, e.g., Bennett, J. W. and LaSure, L. (eds.), More Gene Manipulations in Fungi, Academic Press, CA, 1991). Suitable media are available from commercial suppliers or may be prepared according to published compositions (e.g., in catalogues of the American Type Culture Collection). Temperature ranges and other conditions suitable for growth and enzyme production are known in the art (see, e.g., Bailey, J. E., and Ollis, D. F., Biochemical Engineering Fundamentals, McGraw-Hill Book Company, NY, 1986).

The fermentation can be any method of cultivation of a cell resulting in the expression or isolation of an enzyme or protein. Fermentation may, therefore, be understood as comprising shake flask cultivation, or small- or large-scale fermentation (including continuous, batch, fed-batch, or solid state fermentations) in laboratory or industrial fermentors performed in a suitable medium and under conditions allowing the enzyme to be expressed or isolated. The resulting enzymes produced by the methods described above may be recovered from the fermentation medium and purified by conventional procedures.

Fermentation. The fermentable sugars obtained from the solubilized municipal solid waste can be fermented by one or more (e.g., several) fermenting microorganisms capable of fermenting the sugars directly or indirectly into a desired fermentation product. “Fermentation” or “fermentation process” refers to any fermentation process or any process comprising a fermentation step. Fermentation processes also include fermentation processes used in the consumable alcohol industry (e.g., beer and wine), dairy industry (e.g., fermented dairy products), leather industry, and tobacco industry. The fermentation conditions depend on the desired fermentation product and fermenting organism and can easily be determined by one skilled in the art.

In preferred embodiments, some fermentation will occur concurrent with the hydrolysis of the MSW. Fermentable sugars obtained from the hydrolyzed municipal solid waste material can be fermented by one or more (e.g., several) fermenting microorganisms capable of fermenting the sugars directly or indirectly into fermentation products such as volatile fatty acids (e.g. acetate, propionate, butyrate), lactate and alcohols.

In the fermentation step, sugars, released from the municipal solid waste material as a result of the pretreatment and enzymatic hydrolysis steps, are fermented to a product, e.g., ethanol, by a fermenting organism, such as yeast. Hydrolysis and fermentation can be separate or simultaneous.

Any suitable hydrolyzed municipal solid waste material can be used in the fermentation step in practicing the present invention. The material is generally selected based on economics, i.e., costs per equivalent sugar potential, and recalcitrance to enzymatic conversion.

The term “fermentation medium” is understood herein to refer to a medium before the fermenting microorganism(s) is (are) added, such as, a medium resulting from a saccharification process, as well as a medium used in a simultaneous saccharification and fermentation process (SSF).

“Fermenting microorganism” refers to any microorganism, including bacterial and fungal organisms, suitable for use in a desired fermentation process to produce a fermentation product. The fermenting organism can be hexose and/or pentose fermenting organisms, or a combination thereof. Both hexose and pentose fermenting organisms are well known in the art. Suitable fermenting microorganisms are able to ferment, i.e., convert, sugars, such as glucose, xylose, xylulose, arabinose, maltose, mannose, galactose, and/or oligosaccharides, directly or indirectly into the desired fermentation product. Examples of bacterial and fungal fermenting organisms producing ethanol are described by Lin et al., 2006, Appl. Microbiol. Biotechnol. 69: 627-642.

Examples of fermenting microorganisms that can ferment hexose sugars include bacterial and fungal organisms, such as yeast. Yeast include strains of Candida, Kluyveromyces, and Saccharomyces, e.g., Candida sonorensis, Kluyveromyces marxianus, and Saccharomyces cerevisiae.

Examples of fermenting organisms that can ferment pentose sugars in their native state include bacterial and fungal organisms, such as some yeast. Xylose fermenting yeast include strains of Candida, preferably C. sheatae or C. sonorensis; and strains of Pichia, e.g., P. stipitis, such as P. stipitis CBS 5773. Pentose fermenting yeast include strains of Pachysolen, preferably P. tannophilus. Organisms not capable of fermenting pentose sugars, such as xylose and arabinose, may be genetically modified to do so by methods known in the art.

Examples of bacteria that can efficiently ferment hexose and pentose to ethanol include, for example, Bacillus coagulans, Clostridium acetobutylicum, Clostridium thermocellum, Clostridium phytofermentans, Geobacillus sp., Thermoanaerobacter saccharolyticum, and Zymomonas mobilis (Philippidis, G. P., 1996, Cellulose bioconversion technology, in Handbook on Bioethanol: Production and Utilization, Wyman, C. E., ed., Taylor & Francis, Washington, D.C., 179-212).

Other fermenting organisms include strains of Bacillus, such as Bacillus coagulans; Candida, such as C. sonorensis, C. methanosorbosa, C. diddensiae, C. parapsilosis, C. naedodendra, C. blankii, C. entomophilia, C. brassicae, C. pseudotropicalis, C. boidinii, C. utilis, and C. scehatae; Clostridium, such as C. acetobutylicum, C. thermocellum, and C. phytofermentans; E. coli, especially E. coli strains that have been genetically modified to improve the yield of ethanol; Geobacillus sp.; Hansenula, such as Hansenula anomala; Klebsiella, such as K. oxytoca; Kluyveromyces, such as K. marxianus, K. lactis, K. thermotolerans, and K. fragilis; Schizosaccharomyces, such as S. pombe; Thermoanaerobacter, such as Thermoanaerobacter saccharolyticum; and Zymomonas, such as Zymomonas mobilis.

In an aspect, the fermenting microorganism has been genetically modified to provide the ability to ferment pentose sugars, such as xylose utilizing, arabinose utilizing, and xylose and arabinose co-utilizing microorganisms.

In another aspect, the fermenting organism comprises one or more polynucleotides encoding one or more cellulolytic enzymes, hemicellulolytic enzymes, and accessory enzymes described herein.

It is well known in the art that the organisms described above can also be used to produce other substances, as described herein.

The fermenting microorganism is typically added to the degraded municipal solid waste material or hydrolysate and the fermentation is performed for about 8 to about 96 hours, e.g., about 24 to about 60 hours. The temperature is typically between about 26° C. to about 60° C., e.g., about 32° C. or 50° C., and about pH 3 to about pH 8, e.g., pH 4-5, 6, or 7.

In one aspect, the yeast and/or another microorganism are applied to the degraded municipal solid waste material and the fermentation is performed for about 12 to about 96 hours, such as typically 24-60 hours. In another aspect, the temperature is preferably between about 20° C. to about 60° C., e.g., about 25° C. to about 50° C., about 32° C. to about 50° C., or about 32° C. to about 50° C., and the pH is generally from about pH 3 to about pH 7, e.g., about pH 4 to about pH 7. However, some fermenting organisms, e.g., bacteria, have higher fermentation temperature optima. Yeast or another microorganism is preferably applied in amounts of approximately 10⁵ to 10¹², preferably from approximately 10⁷ to 10¹⁰, especially approximately 2×10⁸ viable cell count per ml of fermentation broth. Further guidance in respect of using yeast for fermentation can be found in, e.g., “The Alcohol Textbook” (Editors K. Jacques, T. P. Lyons and D. R. Kelsall, Nottingham University Press, United Kingdom 1999), which is hereby incorporated by reference.

A fermentation stimulator can be used in combination with any of the processes described herein to further improve the fermentation process, and in particular, the performance of the fermenting microorganism, such as, rate enhancement and ethanol yield. A “fermentation stimulator” refers to stimulators for growth of the fermenting microorganisms, in particular, yeast. Preferred fermentation stimulators for growth include vitamins and minerals. Examples of vitamins include multivitamins, biotin, pantothenate, nicotinic acid, meso-inositol, thiamine, pyridoxine, para-aminobenzoic acid, folic acid, riboflavin, and Vitamins A, B, C, D, and E. See, for example, Alfenore et al., Improving ethanol production and viability of Saccharomyces cerevisiae by a vitamin feeding strategy during fed-batch process, Springer-Verlag (2002), which is hereby incorporated by reference. Examples of minerals include minerals and mineral salts that can supply nutrients comprising P, K, Mg, S, Ca, Fe, Zn, Mn, and Cu.

Biogas. The present invention also relates to processes for producing biogas, comprising the steps of: (a) solubilizing a municipal solid waste (MSW) with the enzyme composition of the present invention (comprising a cellulolytic enzyme composition, a protease, and optionally one or more enzymes from the group cellulase (including endoglucanase, cellobiohydrolase, and/or a beta-glucosidase), an AA9 polypeptide, a beta-glucanase, a cellulose inducing protein, a hemicellulase (including xylanase, acetylxylan esterase, feruloyl esterase, arabinofuranosidase, xylosidase, glucuronidase), an esterase, an expansin, a ligninolytic enzyme, a lipase, a mannanase, an oxidoreductase, a pectinase, and/or a swollenin) in a biogas digester tank to a bioliquid comprising the solubilized material; (b) inoculating the bioliquid comprising the solubilized material of step (a) with one or more microorganisms; and (c) incubating the mixture of step (b) under suitable conditions for production of biogas. The term “biogas” means a gas obtained from a conventional anaerobic fermentor, the primary digester. The main component of biogas is methane and the terms “biogas” and “methane” are in used herein interchangeably.

The process of the present invention increases the degradability of the MSW making it more accessible for a following microbial or biological process such as, for example, a biogas production process leading to a higher yield than would have been possible without the process of the invention.

Fermentation products: A fermentation product can be any substance derived from the fermentation. The fermentation product can be, without limitation, an alcohol (e.g., arabinitol, n-butanol, isobutanol, ethanol, glycerol, methanol, ethylene glycol, 1,3-propanediol [propylene glycol], butanediol, glycerin, sorbitol, and xylitol); an alkane (e.g., pentane, hexane, heptane, octane, nonane, decane, undecane, and dodecane), a cycloalkane (e.g., cyclopentane, cyclohexane, cycloheptane, and cyclooctane), an alkene (e.g., pentene, hexene, heptene, and octene); an amino acid (e.g., aspartic acid, glutamic acid, glycine, lysine, serine, and threonine); a gas (e.g., methane, hydrogen (H₂), carbon dioxide (CO₂), and carbon monoxide (CO)); isoprene; a ketone (e.g., acetone); an organic acid (e.g., acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, oxaloacetic acid, propionic acid, succinic acid, and xylonic acid); and polyketide.

In one aspect, the fermentation product is an alcohol. The term “alcohol” encompasses a substance that contains one or more hydroxyl moieties. The alcohol can be, but is not limited to, n-butanol, isobutanol, ethanol, methanol, arabinitol, butanediol, ethylene glycol, glycerin, glycerol, 1,3-propanediol, sorbitol, xylitol. See, for example, Gong et al., 1999, Ethanol production from renewable resources, in Advances in Biochemical Engineering/Biotechnology, Scheper, T., ed., Springer-Verlag Berlin Heidelberg, Germany, 65: 207-241; Silveira and Jonas, 2002, Appl. Microbiol. Biotechnol. 59: 400-408; Nigam and Singh, 1995, Process Biochemistry 30 (2): 117-124; Ezeji et al., 2003, World Journal of Microbiology and Biotechnology 19 (6): 595-603.

In another aspect, the fermentation product is an alkane. The alkane may be an unbranched or a branched alkane. The alkane can be, but is not limited to, pentane, hexane, heptane, octane, nonane, decane, undecane, or dodecane.

In another aspect, the fermentation product is a cycloalkane. The cycloalkane can be, but is not limited to, cyclopentane, cyclohexane, cycloheptane, or cyclooctane.

In another aspect, the fermentation product is an alkene. The alkene may be an unbranched or a branched alkene. The alkene can be, but is not limited to, pentene, hexene, heptene, or octene.

In another aspect, the fermentation product is an amino acid. The organic acid can be, but is not limited to, aspartic acid, glutamic acid, glycine, lysine, serine, or threonine. See, for example, Richard and Margaritis, 2004, Biotechnology and Bioengineering 87 (4): 501-515.

In another aspect, the fermentation product is a gas. The gas can be, but is not limited to, methane, H₂, CO₂, or CO. See, for example, Kataoka et al., 1997, Water Science and Technology 36 (6-7): 41-47; and Gunaseelan, 1997, Biomass and Bioenergy 13 (1-2): 83-114.

In another aspect, the fermentation product is isoprene.

In another aspect, the fermentation product is a ketone. The term “ketone” encompasses a substance that contains one or more ketone moieties. The ketone can be, but is not limited to, acetone.

In another aspect, the fermentation product is an organic acid. The organic acid can be, but is not limited to, acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, propionic acid, succinic acid, or xylonic acid. See, for example, Chen and Lee, 1997, Appl. Biochem. Biotechnol. 63-65: 435-448.

In another aspect, the fermentation product is polyketide.

Recovery. The fermentation product(s) can be optionally recovered from the fermentation medium using any method known in the art including, but not limited to, chromatography, electrophoretic procedures, differential solubility, distillation, or extraction. For example, alcohol is separated from the fermented MSW and purified by conventional methods of distillation. Ethanol with a purity of up to about 96 vol. % can be obtained, which can be used as, for example, fuel ethanol, drinking ethanol, i.e., potable neutral spirits, or industrial ethanol.

### EXAMPLES

### Example 1: Preparation of a Model Municipal Solid Waste (MSW) Substrate

A model municipal solid waste (MSW) substrate was prepared consisting of the following fractions: 1) a fruit and vegetable fraction, 2) an animal-based and fat fraction, 2) a starch-based fraction and 4) a paper fraction, which were mixed in a 45:10:10:35 ratio (by weight). Each fraction was prepared by thoroughly chopping and mixing (using a food processor) the components listed in the following tables. For some experiments only 1 or 2 of the fractions were used/mixed to constitute the model MSW substrate.

| | Amount | Amount |
|---|---|---|
| I. Fruit and vegetable fraction | [wt %] | at 10 kg |
| Cucumber | 2 | 0.090 kg |
| Orange peel | 4 | 0.180 kg |
| Avocado peel | 2 | 0.090 kg |
| Banana peel | 4 | 0.180 kg |
| Lemons | 1 | 0.045 kg |
| Seedless grapes (red, organic) | 2 | 0.090 kg |
| Peel from honeydew melon | 4 | 0.180 kg |
| Kiwi | 2 | 0.090 kg |
| Strawberry | 3 | 0.135 kg |
| Pear | 3 | 0.135 kg |
| Tomato | 4 | 0.180 kg |
| Apple | 5 | 0.225 kg |
| Pepper/capsicum stalk | 4 | 0.180 kg |
| Broccoli stalk | 6 | 0.270 kg |
| Field mushroom | 5 | 0.225 kg |
| Carrot peel | 10 | 0.450 kg |
| Iceberg lettuce (organic) | 5 | 0.225 kg |
| Potato peel | 1 | 0.450 kg |
| onion peel | 3 | 0.135 kg |
| Leek | 4 | 0.180 kg |
| Haricots verts (boiled) | 3 | 0.135 kg |
| Broccoli florets (boiled) | 5 | 0.225 kg |
| White cabbage | 5 | 0.225 kg |
| Peas (boiled) | 4. | 0.180 kg |
| Sum | 100% | 4.500 kg |

| II: Animal-based and fats fraction | At 10 kg | |
|---|---|---|
| A38 minimilk (fermented dairy product sold in | 8% | 0.081 kg |
| Denmark) | | |
| Sliced cheese (mild) 45+, (fat content of 45% in the | 8% | 0.081 kg |
| dried cheese) | | |
| Meatballs (Danish frikadeller) | 8% | 0.081 kg |
| Smoked salmon | 3% | 0.032 kg |
| Boiled egss with eggshell | 3% | 0.032 kg |
| Bolognese sauce | 8% | 0.081 kg |
| Liver paste (The Danish National spread | 8% | 0.081 kg |
| ‘leverpostej’) | | |
| Tenderloin pot, ready meal (Danish dish | 8% | 0.081 kg |
| ‘mørbradgryde’) | | |
| Beef salami | 5% | 0.048 kg |
| Danish salami, 3-star | 5% | 0.048 kg |
| Chicken breast, roasted | 5% | 0.048 kg |
| Tuna in oil (canned food) | 5% | 0.048 kg |
| Roasted beef (not minced meat) | 8% | 0.081 kg |
| Fish fillet | 8% | 0.081 kg |
| Oil | 5% | 0.048 kg |
| Butter | 5% | 0.048 kg |
| Sum | 100% | 1.00 kg |

| | |
|---|---|
| III: Starch-based fraction | At 10 kg |

| | | |
|---|---|---|
| Basmati rice (boiled) | 15% | 0.154 kg |
| Cake | 8% | 0.077 kg |
| Toast bread | 15% | 0.154 kg |
| Pasta (boiled) | 15% | 0.154 kg |
| Oat (organic) | 15% | 0.154 kg |
| Rye bread | 15% | 0.154 kg |
| Potatoes (boiled) | 15% | 0.154 kg |
| Sum | 100% | 1.00 kg |

| IV: Paper fraction | At 10 kg | |
|---|---|---|
| Advertisements | 7.6% | 0.265 kg |
| Books & booklets | 1.0% | 0.036 kg |
| Magazines & journals | 1.6% | 0.057 kg |
| Newspapers | 2.7% | 0.093 kg |
| Office paper | 2.3% | 0.079 kg |
| Tissue paper | 16.2% | 0.567 kg |
| Envelopes | 0.6% | 0.022 kg |
| Kraft paper | 0.2% | 0.007 Kg |
| Wrapping paper | 0.2% | 0.007 Kg |
| Other paper | 0.6% | 0.022 Kg |
| Corrugated boxes | 2.3% | 0.079 Kg |
| Folding boxes | 7.2% | 0.251 Kg |
| Beverage cartons | 16.2% | 0.567 Kg |
| Egg boxes | 0.4% | 0.014 Kg |
| Cards & labels | 0.4% | 0.014 Kg |
| Board tubes | 1.2% | 0.043 Kg |
| Other board | 0.6% | 0.022 Kg |
| Human hygiene waste (diapers, cotton . . .) | 26.2% | 0.918 Kg |
| Wood untreated | 1.8% | 0.065 Kg |
| Textiles, leather and rubber (tea towels) | 10.7% | 0.373 Kg |
| Sum | 100% | 3.500 Kg |

The recipe for the model MSW substrate is based on Luca Alibardi and Raffaello Cossu, Composition variability of the organic fraction of municipal solid waste and effects on hydrogen and methane production potentials, Waste Management 36: 147-155 (2015); S. Lebersorger and F. Schneider, Discussion on the methodology for determining food waste in household waste composition studies, Waste Management 31: 1924-1933 (2011); and Edjabou et al., Municipal solid waste composition: Sampling methodology, statistical analyses and case study evaluation, Waste Management 36: 12-23 (2015).

### Example 2: Assay for Enzymatic Solubilization of a Model Municipal Solid Waste (MSW) Substrate

Experiments were performed on a 10 g scale in 50 mL centrifuge tubes with 50 mM acetic acid buffer at a TS (total solid) concentration of 7.5%. Enzymes were added to a cellulolytic enzyme composition and incubated at 50° C., pH 5 for 24 hours at 4 rpm in Stuart Rotator. Enzyme performance was measured as the ability of the enzymes to solubilize a model MSW substrate. The cellulolytic enzyme composition without any of the added enzymes was used as control and dosed at 2.5% product/total solid(TS). More specifically, the assay was conducted as follows:
1. Model MSW was mixed into a homogeneous mass and weighed into 50 mL Falcon tubes.
2. Buffer (sodium acetate buffer (0.050M, pH 5)) was added and the samples were shaken and incubated overnight at 50° C.
3. The enzyme composition was diluted and added to the tubes.
4. MilliQ water was added to reach a total reaction mass of 10 g and the samples were mixed vigorously.
5. The tubes were then placed on a Stuart Rotator (4 rpm) in a heating oven at 50 ° C.
6. The tubes were incubated for 24 hours.
7. The weight of filter tubes was determined before use.
8. Following incubation, the total reaction mass was transferred to a filter tube and centrifuged for 10 minutes at 4000 rpm
9. The supernatant was poured off.
10. The filter tubes with remaining pellets were dried at 60° C. in a heating oven for two days.
11. Following drying, the weight of all tubes was noted.
12. The transfer of solids from the substrate to the liquid phase was calculated to express the hydrolysis efficiency as conversion of total solids (percentage).

### Example 3: The Solubilization Effect of Various Proteases on a Model MSW Substrate

Screening of several proteases added to a cellulolytic enzyme composition supplemented with a Bacillus bogoriensis mannanase of SEQ ID NO: 3 in a ratio of 95:5 enzyme protein (designated herein as “cellulolytic enzyme composition CEC/M”) was performed to select further candidates using the MSW assay described In Example 2. The cellulolytic enzyme composition (designated herein as “cellulolytic enzyme composition CEC”) was derived from Trichoderma reesei further comprising the cellobiohydrolase I of the mature polypeptide of SEQ ID NO: 15, the cellobiohydrolase II of the mature polypeptide of SEQ ID NO: 16, the beta-glucosidase of the mature polypeptide of SEQ ID NO: 17, the AA9 (GH61) polypeptide having cellulolytic enhancing activity of the mature polypeptide of SEQ ID NO: 18, the xylanase of the mature polypeptide of SEQ ID NO: 21, and the beta-xylosidase of the mature polypeptide of SEQ ID NO: 22.

The screening was performed on a 10 g scale in 50 ml Corning tubes where a model MSW substrate composed of the animal-based and fats fraction (Example 1) was hydrolyzed by cellulolytic enzyme composition CEC/M with and without each of the different proteases. The substrate was mixed with 50 mM citric acid buffer to a final TS of 1.5%. The cellulolytic enzyme composition was added in each trial at an amount of 0.5% EP/TS. Each protease was mixed on top of the cellulolytic enzyme composition at 0.02% EP/TS. The same amount of cellulolytic enzyme composition CEC/M without protease was also tested as control. The tubes were incubated for 24 hours at 50° C. on a Stuart Rotator (4 rpm). Subsequently, liquid and solid were separated by centrifugation filtration. Then the weight of the remaining solid before and after drying at 60° C. for 2 days was determined. The concentrations of the proteases used in the assay are listed below:

| Proteases | Source | SEQ ID NO | (g/l) |
|---|---|---|---|
| 1 | Bacillus clausii | SEQ ID NO: 32 | 59.1 |
| 2 | Bacillus clausii | SEQ ID NO: 32 + (Y161A + | 55.2 |
| | | R164S + A188P) | |
| 3 | Bacillus clausii | SEQ ID NO: 32 + (M216S) | 62.9 |
| 4 | Bacillus clausii | SEQ ID NO: 32 + S97AD (insertion | 144.0 |
| | | of D and substitution of S with A | |
| | | at position 97 in the back bone) | |
| 5 | Bacillus clausii | SEQ ID NO: 32 + (V66A + S104A) | 20.5 |
| 6 | Bacillus | SEQ ID NO: 33 | 62.9 |
| | licheniformis | | |
| 7 | Thermoascus | SEQ ID NO: 1 | 20 |
| | aurantiacus | | |
| 8 | Pyrococcus | SEQ ID NO: 5 | 45.1 |
| | furiosus | | |

The Thermoascus aurantiacus protease of SEQ ID NO: 1 was prepared according to WO 2003/048353. The Pyrococcus furiosus protease of SEQ ID NO: 5 was prepared according to U.S. Pat. No. 6,358,726-B1. The Bacillus clausii protease of SEQ ID NO: 32 and its variants were prepared according to WO 2011/036263, WO 1998/020115 and WO 2003/006602. Bacillus licheniformis protease of SEQ ID NO: 32 and its variants were prepared according to WO2015/144936 and WO2015/144782

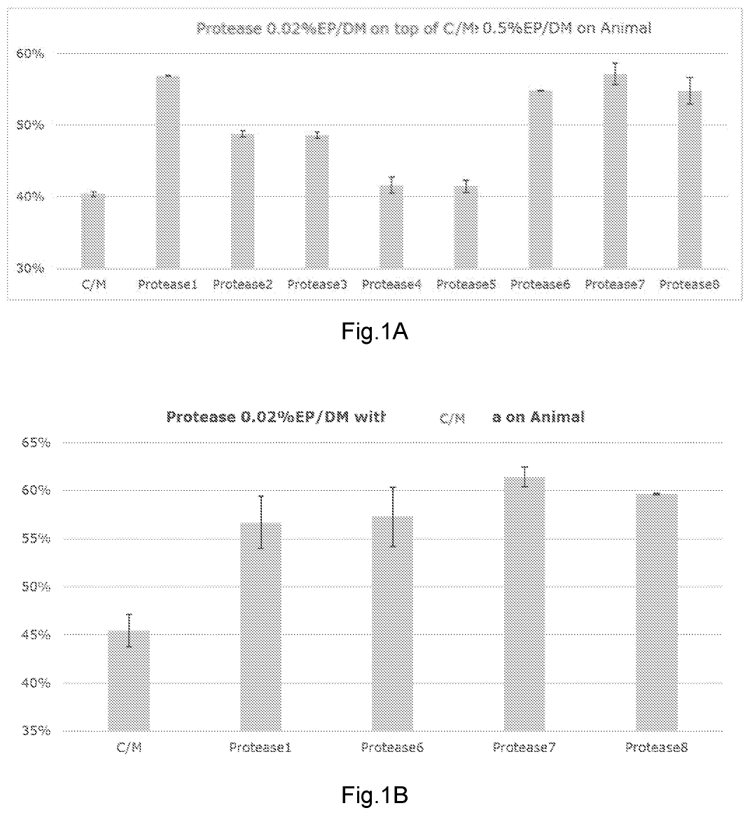

The results are shown in FIG. 1A and FIG. 1B. FIG. 1A demonstrates that proteases 1, 6, 7, and 8 provided a better solubilization effect of the model MSW substrate than the other proteases. As top candidates, the proteases were submitted to a second-round of screening, which was conducted under the same conditions as described above. The results of the second-round screening are shown in FIG. 1B. At a dosing of 0.02 EP/TS of the base enzyme, protease 7 resulted in 61.4% solubilization, which was higher than what was obtained using cellulolytic enzyme composition CEC/M (44.5%), protease 1 (56.7%), protease 6 (57.3%) and protease 8 (59.6%).

The Meripilus giganteus protease of SEQ ID NO: 36 (protease 9) was prepared according to WO 2014/037438 and screened with protease 7, under the same conditions described above, except the CEC/M was added at an amount of 0.45% EP/TS. The results of this third screening are as follows. At a dosing of 0.02% EP/TS of the base enzyme, protease 7 resulted in 40.4% solubilization, and protease 9 resulted in 33.8% solubilization. At a dosing of 0.2% EP/TS of the base enzyme, protease 7 resulted in 44.0% solubilization, and protease 9 resulted in 39.5% solubilization. Both protease 7 and protease 9 had higher solubilization than what was obtained using cellulolytic enzyme composition CEC/M (30.3%).

### Example 4. Dose Response of Proteases on Solubilization of a Model MSW Substrate

This study was designed to select the preferable dose of protease for solubilizing a model MSW substrate composed of the Fruit and Vegetable fraction, Paper fraction, Animal fraction, and Starch fraction in a 45:35:10:10 weight ratio (Example 1). The experiments were performed in 50 ml tubes on a 10 g scale with a dry matter content of 7.5% TS. Cellulolytic enzyme composition CEC/M (Example 3) was added in an amount of 2.5% product/TS and was replaced by a B.a protease (Bacillus amyloliquefaciens protease of SEQ ID NO: 34) and Protease 7, respectively, at different ratios based on enzyme protein. Cellulolytic enzyme composition CEC/M without the proteases was run as a control.

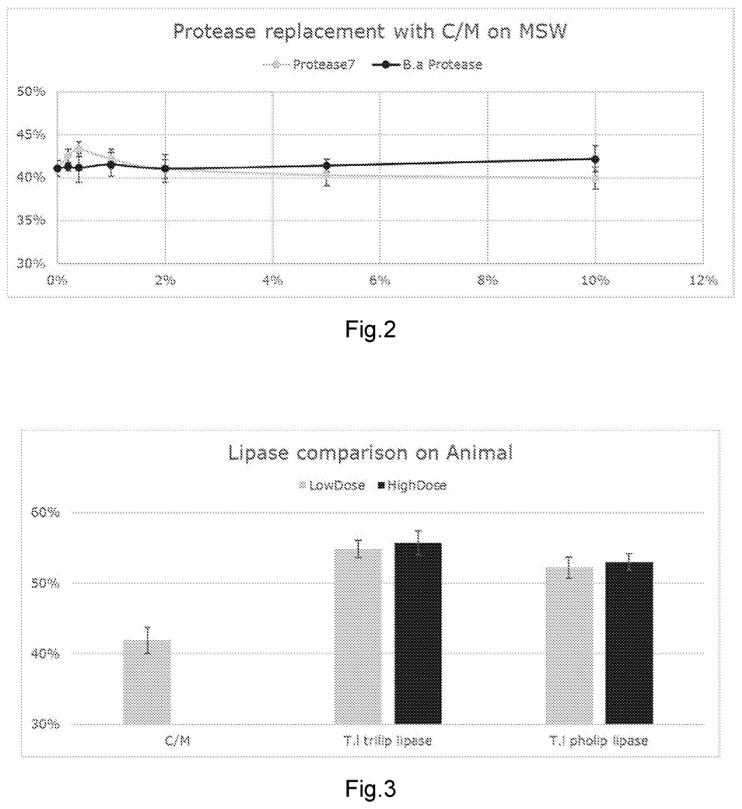

The results shown in FIG. 2 demonstrate that protease 7 provided a boosting effect compared to the B.a protease when the ratio was <2%. For example, when the protease ratio was 0.2%, protease 7 resulted in 42.6% conversion, higher than that of B.a protease (41.3%). The best performance was achieved when the ratio of protease 7 was 0.4%. Here the solubilization level was 43.4%, which was higher than the solubilization level of B.a protease at the same ratio (41.4%).

For B.a protease the solubilization level positively correlated to the dose, i.e., the more B.a protease used, the higher solubilization achieved. On the contrary, protease 7 did not show such a correlation and the peak of solubilization was achieved when the ratio was 0.4%.

### Example 5. Optimization of an Enzyme Composition for Solubilization of a Model MSW Substrate

Statistical experiments were set up to determine the optimal ratio between cellulolytic enzyme composition CEC/M and the selected enzyme candidates in a multicomponent enzymes blend.

Selection for suitable lipase. The experiments were performed to select the most suitable lipase. Two lipases were tested: (1) T.l trilip lipase (SEQ ID NO: 2): A triacylglycerol lipase derived from Thermomyces lanuginosus, obtained according to WO 2000/006003; and 2) T.l pholip lipase (SEQ ID NO: 35): A triacylglycerol lipase with phospholipase activity derived from Thermomyces lanuginosus. Specifically, experiments were performed in 50 ml tubes on a 10 g scale with a dry matter content of 1.5% TS using a model MSW substrate based solely on the animal fraction (Example 1). Cellulolytic enzyme composition CEC/M (Example 3) was added in an amount of 2.5% product/TS and each lipase was loaded at 0.1% (low dose) and 1% (high dose) product/TS on top of base enzyme, respectively. Cellulolytic enzyme composition CEC/M without the lipases was run as a control.

The results as shown in FIG. 3 demonstrate that the best solubilization improvement over cellulolytic enzyme composition CEC/M was achieved using the T.l trilip lipase at the high dose (Solubilization level of 55.7%). Even at a low dose, the T.l trilip lipase results in a solubilization level of 54.9%, which was better than the other lipases regardless of dosing conditions.

Dose optimization of pectinase. The pectinase used in the present invention was derived from Aspergillus aculeatus in wild type (A. aculeatus pectinase). Experiments were performed in 50 ml tube on a 10 g scale with a dry matter content of 6% TS using the Fruit and Vegetable fraction an Paper fraction (Example 1) in a 45:35 weight ratio. Cellulolytic enzyme composition CEC/M was added in an amount of 2.5% product/TS and was replaced by A. aculeatus pectinase at different ratio based on enzyme protein and the standard assay for enzymatic solubilization was performed.

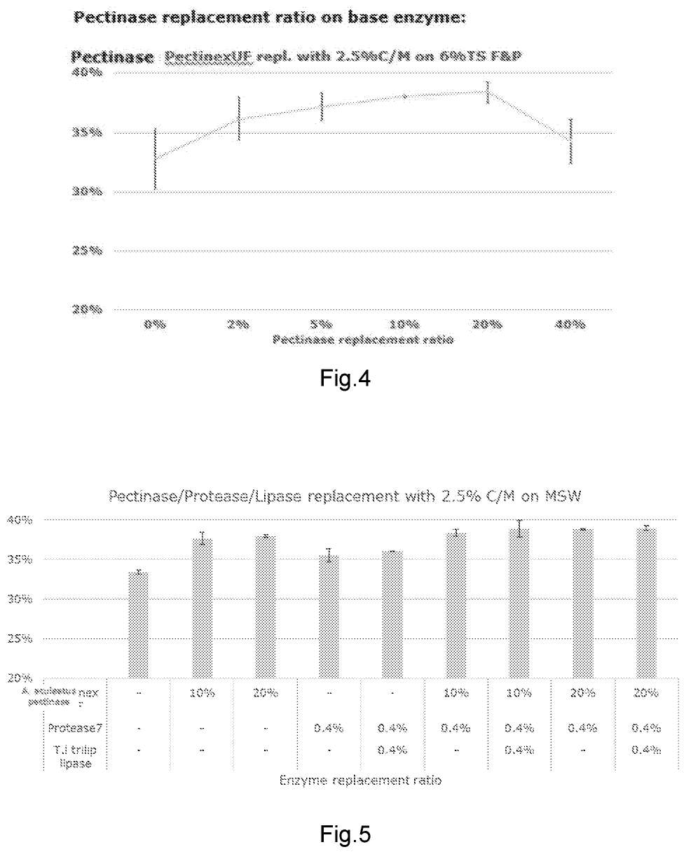

The results (FIG. 4) show that the optimum ratio for A. aculeatus pectinase was around 10-20% replacement of the base enzyme, reaching a solubilization level of 38%.

Optimization of the enzyme composition. Statistical experiments were performed to determine the optimal ratio between cellulolytic enzyme composition CEC/M and the selected enzyme candidates in a multicomponent enzyme blend. Experiments were performed in 50 ml tubes on a 10 g scale with cellulolytic enzyme composition CEC/M at a concentration of 2.5% product/TS using a model MSW substrate composed of the Fruit and Vegetable fraction, Paper fraction, Animal fraction, and Starch fraction in a 45:35:10:10 weight ratio (Example 1). The cellulolytic enzyme composition CEC/M was added in an amount of 2.5% product/TS and was replaced by other enzymes based on enzyme protein. A comparison assay was conducted for the solubilization effect of the enzyme composition and the results are shown in FIG. 5. Overall, the best performance was observed with A. aculeatus pectinase, protease 7, T.l trilip lipase and cellulolytic enzyme composition CEC/M in a EP weight ratio of 20:0.4:0.4:79.2. Solubilization of the model MSW substrate was improved from 33% with cellulolytic enzyme composition CEC/M to 39% at the default enzyme dose (2.5% product/TS).

Synergy effect of the enzyme composition. Experiments were performed to test the efficiency of the selected enzymes composition (see table below). The experiments were performed in 50 ml tubes on a 10 g scale at a dry matter content of 7.5% TS using a model MSW substrate composed of the Fruit and Vegetable fraction, Paper fraction, Animal fraction, and Starch fraction in a 45:35:10:10 weight ratio. Cellulolytic enzyme composition CEC/M, Sample 1, and Sample 2 were added in amount of 2.5%, 5%, 7.5% and 15% product/TS.

| cellulolytic | the cellulolytic | | |
|---|---|---|---|
| enzyme | enzyme | | |
| composition | composition | | |
| CEC | CEC/M | Sample 1 | Sample 2 |
| (ratio) | (ratio) | (ratio) | (ratio) |
| CEC (100) | CEC (95) | CEC/M (70) | CEC/M (75.2) |
| | mannanase (5) | A. aculeatus | mannanase (4) |
| | | beta- | |
| | | glucanase (15) | |
| | | B.a protease | A. aculeatus |
| | | (10) | pectinase (20) |
| | | T.l pholip | Protease 7 (0.4) |
| | | lipase (5) | |
| | | | T.l trilip |
| | | | lipase (0.4) |

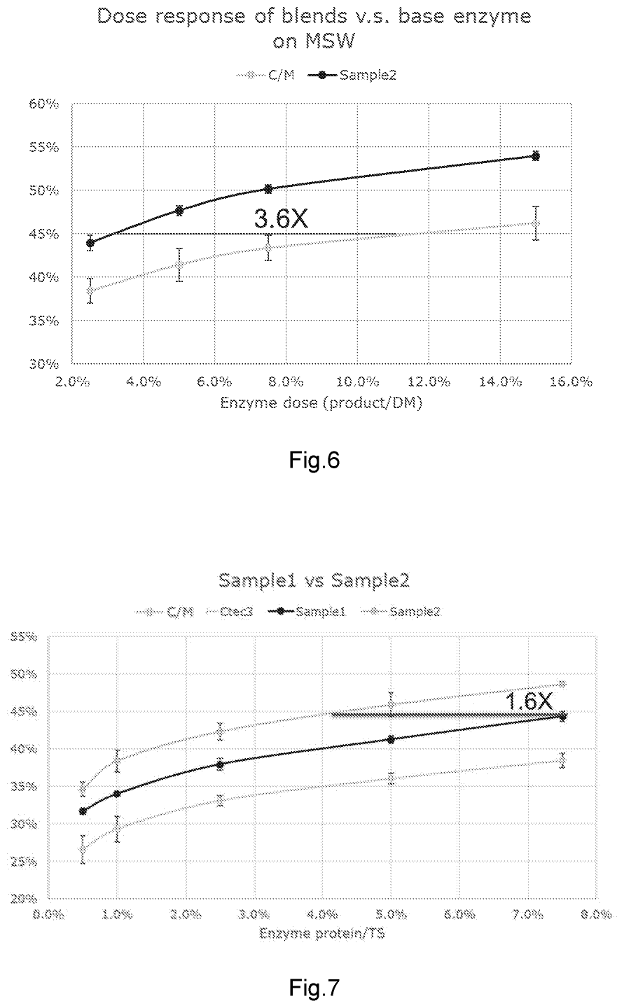

FIG. 6 shows the dose/response curves for cellulolytic enzyme composition CEC/M and Sample 2. A significant improvement in TS solubilization was observed at all applied enzyme concentration for Sample 2 compared to cellulolytic enzyme composition CEC/M. The solubilization achieved with Sample 2 was 44% at 2.5% product/TS and the dose of cellulolytic enzyme composition CEC/M required to achieve the same degree of solubilization is 9% product/TS; hence a factor of 3.6 higher.

FIG. 7 shows the dose/response curves for cellulolytic enzyme composition CEC, Sample 1, and Sample 2. A significant improvement in TS solubilization was observed at all applied enzyme concentrations for Sample 2 compared to Sample 1. The solubilization for Sample 2 was 45.3% at 5% product/TSM and the dose of Sample 1 required to achieve a similar solubilization (44.6%). was 7.5% product/TS; hence 1.6 times higher.

1. An enzyme composition for solubilizing a municipal solid waste, said composition comprising: (i) a cellulolytic enzyme composition, and (ii) a protease selected from the group consisting of: (a) a protease having at least 50%, identity to amino acids 1 to 177 of SEQ ID NO: 1 or a fragment thereof having protease activity; (b) a protease having at least 50% identity identity to SEQ ID NO: 5; (c) a protease having at least 50% identity to SEQ ID NO: 32 or a variant thereof; (d) a protease having at least 50% identity to SEQ ID NO: 33; and (e) a protease having at least 50% identity to amino acids 199 to 564 of SEQ ID NO: 36.

2. The enzyme composition of claim 1, which further comprises one or more enzymes selected from the group consisting of a cellulase, an AA9 polypeptide, a beta-glucanase, a cellulose inducing protein, a hemicellulase, an esterase, an expansin, a ligninolytic enzyme, a lipase, a mannanase, an oxidoreductase, a pectinase, and a swollenin.

3. (canceled)

4. (canceled)

5. (canceled)

6. The enzyme composition of claim 1, wherein the cellulolytic enzyme composition comprises (a) an endoglucanase I, (b) an endoglucanase II, (c) a cellobiohydrolase I or variant thereof; (d) a cellobiohydrolase II or variant thereof; (e) a beta-glucosidase or variant thereof; (f) an AA9 polypeptide having cellulolytic enhancing activity; and optionally (g) a xylanase and/or (h) a beta-xylosidase.

7. The enzyme composition of claim 1, wherein the cellulolytic enzyme composition derived from Trichoderma reesei further comprises the cellobiohydrolase I of SEQ ID NO: 15, the cellobiohydrolase II of SEQ ID NO: 16, the beta-glucosidase of SEQ ID NO: 17, the AA9 (GH61) polypeptide having cellulolytic enhancing activity of SEQ ID NO: 18, the xylanase of SEQ ID NO: 21, and the beta-xylosidase of SEQ ID NO: 22.

8. (canceled)

9. The enzyme composition of claim 2, wherein the lipase has at least 50% identity with SEQ ID NO: 2, wherein the mannanase is an endo-mannosidase with at least 50% identity with SEQ ID NO: 3, and/or wherein the beta-glucanase has at least 50% identity with SEQ ID NO: 4.

10. (canceled)

11. (canceled)

12. The enzyme composition of claim 1, wherein the protease is present at a ratio between 0.1 -2% w/w of the total enzyme protein, wherein the lipase is present at a ratio between 0-10% w/w of the total enzyme, wherein the mannanase is present at a ratio between 0-10% w/w of the total enzyme protein, wherein the pectinase is present at a ratio between 0-30% w/w of the total enzyme protein, and/or wherein the beta-glucanase is present at a ratio between 0-30% w/w of the total enzyme protein.

13-16. (canceled)

17. The enzyme composition of claim 1, wherein the cellulolytic enzyme composition is present at a ratio between 40%-99% w/w of the total enzyme protein.

18. A process for solubilizing a municipal solid waste, comprising treating the municipal solid waste with the enzyme composition of claim 1 to produce a solubilized municipal solid waste and optionally recovering the solubilized municipal solid waste.

19. The process of claim 18, wherein the solubilized municipal solid waste is a sugar.

20. A process for producing a fermentation product, comprising: (a) solubilizing a municipal solid waste with the enzyme composition of claim 1; (b) fermenting the solubilized municipal solid waste with one or more fermenting microorganisms to produce the fermentation product; and (c) recovering the fermentation product from the fermentation.

21-25. (canceled)

26. A process for producing biogas, comprising the steps of:
(a) solubilizing a municipal solid waste (MSW) with the enzyme composition of claim 1 in a biogas digester tank;
(b) inoculating the solubilized MSW of step (a) with one or more microorganisms; and
(c) incubating the mixture of step (b) under suitable conditions for production of biogas.

27. A process for solubilizing a municipal solid waste, comprising treating the municipal solid waste with the components of the enzyme composition of claim 1, wherein the cellulolytic enzyme composition and the protease are added separately, to produce a solubilized municipal solid waste and optionally recovering the solubilized municipal solid waste.

28. A process for solubilizing a municipal solid waste, comprising treating the municipal solid waste with an effective amount of (i) a cellulolytic enzyme composition, and (ii) a protease selected from the group consisting of: (a) a protease having at least 50% identity to amino acids 1 to 177 of SEQ ID NO: 1 or a fragment thereof having protease activity; (b) a protease having at least 50% identity to SEQ ID NO: 5; (c) a protease having at least 50% identity to SEQ ID NO: 32 or a variant thereof; (d) a protease having at least 50% identity to SEQ ID NO: 33; and (e) a protease having at least 50% identity to amino acids 199 to 564 of SEQ ID NO: 36, to produce a solubilized municipal solid waste, and optionally recovering the solubilized municipal solid waste.

29. The process of claim 28, further comprising treating the municipal solid waste with one or more enzymes selected from a cellulase, an AA9 polypeptide, a beta-glucanase, a cellulose inducing protein, a hemicellulase, an esterase, an expansin, a ligninolytic enzyme, a lipase, a mannanase, an oxidoreductase, a pectinase, and a swollenin.

30. (canceled)

31. (canceled)

32. (canceled)

33. The process of claim 28, wherein the cellulolytic enzyme composition comprises (a) an endoglucanase I, (b) an endoglucanase II, (c) a cellobiohydrolase I or variant thereof; (d) a cellobiohydrolase II or variant thereof; (e) a beta-glucosidase or variant thereof; (f) an AA9 polypeptide having cellulolytic enhancing activity; and optionally (g) a xylanase and/or (h) a beta-xylosidase.

34. The process of claim 28, wherein the cellulolytic enzyme composition derived from Trichoderma reesei further comprises the cellobiohydrolase I of SEQ ID NO: 15, the cellobiohydrolase II of SEQ ID NO: 16, the beta-glucosidase of SEQ ID NO: 17, the AA9 (GH61) polypeptide having cellulolytic enhancing activity of SEQ ID NO: 18, the xylanase of SEQ ID NO: 21, and the beta-xylosidase of SEQ ID NO: 22.

35. (canceled)

36. The process of claim 32, wherein the lipase has at least 50% identity with SEQ ID NO: 2, wherein the mannanase is an endo-mannosidase with at least 50% identity with SEQ ID NO: 3, and/or wherein the beta-glucanase has at least 50% identity with SEQ ID NO: 4.

37. (canceled)

38. (canceled)

39. The process of claim 28, wherein the protease is present at a ratio between 0.1-2% w/w of the total enzyme protein, wherein the lipase is present at a ratio between 0-10% w/w of the total enzyme, wherein the mannanase is present at a ratio between 0-10% w/w of the total enzyme protein, wherein the pectinase is present at a ratio between 0-30% w/w of the total enzyme protein, and/or wherein the beta-glucanase is present at a ratio between 0-30% w/w of the total enzyme protein.

40-43. (canceled)

44. The process of claim 28, wherein the cellulolytic enzyme composition is present at a ratio between 40%-99% w/w of the total enzyme protein.

45. An enzyme composition for solubilizing a municipal solid waste, said composition comprising: (i) a cellulolytic enzyme composition; (ii) a protease selected from the group consisting of: (a) a protease having at least 50% identity to amino acids 1 to 177 of SEQ ID NO: 1 or a fragment thereof having protease activity; (b) a protease having at least 50% identity to SEQ ID NO: 5; (c) a protease having at least 50% identity to SEQ ID NO: 32 or a variant thereof; (d) a protease having at least 50% identity to SEQ ID NO: 33; and (e) a protease having at least 50% identity to amino acids 199 to 564 of SEQ ID NO: 36; and (iii) a mannanase.

46. A process for solubilizing a municipal solid waste, comprising treating the municipal solid waste with (i) a cellulolytic enzyme composition; (ii) a protease selected from the group consisting of: (a) a protease having at least 50% identity to amino acids 1 to 177 of SEQ ID NO: 1 or a fragment thereof having protease activity; (b) a protease having at least 50% identity to SEQ ID NO: 5; (c) a protease having at least 50% identity to SEQ ID NO: 32 or a variant thereof; (d) a protease having at least 50% identity to SEQ ID NO: 33; and (e) a protease having at least 50% identity to amino acids 199 to 564 of SEQ ID NO: 36; and (iii) a mannanase, to produce a solubilized municipal solid waste, and optionally recovering the solubilized municipal solid waste.


SEQUENCE LISTING (36 sequences)

SEQ ID NO: 1 - protein, 355 aa, Thermoascus aurantiacus
  SIGNAL (1)..(19)
  PROPEP (20)..(178)
  mat_peptide (179)..()
MRLVASLTALVALSVPVFPAAVNVKRASSYLEITLSQVSNTLIKAVVQNTGSDELSFVHLNFFKDPAPVKKVSVYRDGSEVQFEGILSRYKSTGLSRDAFTYLAPGESVEDVFDIASTYDLTSGGPVTIRTEGVVPYATANSTDIAGYISYSSNVLTIDVDGAAAATVSKAITPLDRRTRISSCSGSRQSALTTALRNAASLANAAADAAQSGSASKFSEYFKTTSSSTRQTVAARLRAVAREASSSSSGATTYYCDDPYGYCSSNVLAYTLPSYNIIANCDIFYTYLPALTSTCHAQDQATTALHEFTHAPGVYSPGTDDLAYGYQAAMGLSSSQAVMNADTYALYANAIYLGC

SEQ ID NO: 2 - protein, 291 aa, Thermomyces lanuginosus
MRSSLVLFFVSAWTALASPIRREVSQDLFNQFNLFAQYSAAAYCGKNNDAPAGTNITCTGNACPEVEKADATFLYSFEDSGVGDVTGFLALDNTNKLIVLSFRGSRSIENWIGNLNFDLKEINDICSGCRGHDGFTSSWRSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHSSPEYWIKSGTLVPVRRRDIVKIEGIDATGGNNQPNIPDIPAHLWYFGLIGTCL

SEQ ID NO: 3 - protein, 309 aa, Bacillus bogoriensis
ANSGFYVSGTTLYDANGNPFVMRGINHGHAWYKDQATTAIEGIANTGANTVRIVLSDGGQWTKDDIHTVRNLISLAEDNHLVAVLEVHDATGYDSIASLNRAVDYWIEMRSALIGKEDTVIINIANEWFGSWEGDAWADGYKQAIPRLRNAGLNHTLMVDAAGWGQFPQSIHDYGREVFNADPQRNTMFSIHMYEYAGGNASQVRTNIDRVLNQDLALVIGEFGHRHTNGDVDEATIMSYSEQRGVGWLAWSWKGNGPEWEYLDLSNDWAGNNLTAWGNTIVNGPYGLRETSRLSTVFTGGGSDGGTSP

SEQ ID NO: 4 - protein, 245 aa, Aspergillus aculeatus
VPMGSRTKNLATRATNAVVSVSSLAATTLKDNDGSGAGQDVYTFHTGDGSVADGWPAQSSWVSFDDMWKANKPTIMESCTQFGVPNNSANETQNLYDAIQQVAKESHLDHRFILAIIMQESKGCVRVHTTNYGVRNPGLMQDHDGAGTCNDNGVVQNPCPKNEILQMVRDGAIGTAAGDGLASLIDQQGKTDVSGFYRAARLYNSGSISDASNLNVGVGTACYATDVANRLTGWVNAASKCTLSA

SEQ ID NO: 5 - protein, 412 aa, Pyrococcus furiosus
  mat_peptide (1)..(412): Pyrococcus furiosus protease (Pfu)
AELEGLDESAAQVMATYVWNLGYDGSGITIGIIDTGIDASHPDLQGKVIGWVDFVNGRSYPYDDHGHGTHVASIAAGTGAASNGKYKGMAPGAKLAGIKVLGADGSGSISTIIKGVEWAVDNKDKYGIKVINLSLGSSQSSDGTDALSQAVNAAWDAGLVVVVAAGNSGPNKYTIGSPAAASKVITVGAVDKYDVITSFSSRGPTADGRLKPEVVAPGNWIIAARASGTSMGQPINDYYTAAPGTSMATPHVAGIAALLLQAHPSWTPDKVKTALIETADIVKPDEIADIAYGAGRVNAYKAINYDNYAKLVFTGYVANKGSQTHQFVISGASFVTATLYWDNANSDLDLYLYDPNGNQVDYSYTAYYGFEKVGYYNPTDGTWTIKVVSYSGSANYQVDVVSDGSLSQPGSS

SEQ ID NO: 6 - protein, 326 aa, Thielavia terrestris
MKSFTIAALAALWAQEAAAHATFQDLWIDGVDYGSQCVRLPASNSPVTNVASDDIRCNVGTSRPTVKCPVKAGSTVTIEMHQQPGDRSCANEAIGGDHYGPVMVYMSKVDDAVTADGSSGWFKVFQDSWAKNPSGSTGDDDYWGTKDLNSCCGKMNVKIPEDIEPGDYLLRAEVIALHVAASSGGAQFYMSCYQLTVTGSGSATPSTVNFPGAYSASDPGILINIHAPMSTYVVPGPTVYAGGSTKSAGSSCSGCEATCTVGSGPSATLTQPTSTATATSAPGGGGSGCTAAKYQQCGGTGYTGCTTCASGSTCSAVSPPYYSQCL

SEQ ID NO: 7 - protein, 478 aa, Thielavia terrestris
MRFDALSALALAPLVAGHGAVTSYIIGGKTYPGYEGFSPASSPPTIQYQWPDYNPTLSVTDPKMRCNGGTSAELSAPVQAGENVTAVWKQWTHQQGPVMVWMFKCPGDFSSSHGDGKGWFKIDQLGLWGNNLNSNNWGTAIVYKTLQWSNPIPKNLAPGNYLIRHELLALHQANTPQFYAECAQLVVSGSGSALPPSDYLYSIPVYAPQNDPGITVDIYNGGLTSYTPPGGPVWSGFEFMRFDALSALALAPLVAGHGAVTSYIIGGKTYPGYEGFSPASSPPTIQYQWPDYNPTLSVTDPKMRCNGGTSAELSAPVQAGENVTAVWKQWTHQQGPVMVWMFKCPGDFSSSHGDGKGWFKIDQLGLWGNNLNSNNWGTAIVYKTLQWSNPIPKNLAPGNYLIRHELLALHQANTPQFYAECAQLVVSGSGSALPPSDYLYSIPVYAPQNDPGITVDIYNGGLTSYTPPGGPVWSGFEF

SEQ ID NO: 8 - protein, 516 aa, Thielavia terrestris
MLLTSVLGSAALLASGAAAHGAVTSYIIAGKNYPGYQGFSPANSPNVIQWQWHDYNPVLSCSDSKLRCNGGTSATLNATAAPGDTITAIWAQWTHSQGPILVWMYKCPGSFSSCDGSGAGWFKIDEAGFHGDGVKVFLDTENPSGWDIAKLVGGNKQWSSKVPEGLAPGNYLVRHELIALHQANNPQFYPECAQVVITGSGTAQPDASYKAAIPGYCNQNDPNIKVPINDHSIPQTYKIPGPPVFKGTASKKARDFTAMLLTSVLGSAALLASGAAAHGAVTSYIIAGKNYPGYQGFSPANSPNVIQWQWHDYNPVLSCSDSKLRCNGGTSATLNATAAPGDTITAIWAQWTHSQGPILVWMYKCPGSFSSCDGSGAGWFKIDEAGFHGDGVKVFLDTENPSGWDIAKLVGGNKQWSSKVPEGLAPGNYLVRHELIALHQANNPQFYPECAQVVITGSGTAQPDASYKAAIPGYCNQNDPNIKVPINDHSIPQTYKIPGPPVFKGTASKKARDFTA

SEQ ID NO: 9 - protein, 452 aa, Thielavia terrestris
MLANGAIVFLAAALGVSGHYTWPRVNDGADWQQVRKADNWQDNGYVGDVTSPQIRCFQATPSPAPSVLNTTAGSTVTYWANPDVYHPGPVQFYMARVPDGEDINSWNGDGAVWFKVYEDHPTFGAQLTWPSTGKSSFAVPIPPCIKSGYYLLRAEQIGLHVAQSVGGAQFYISCAQLSVTGGGSTEPPNKVAFPGAYSATDPGILINIYYPVPTSYQNPGPAVFSCMLANGAIVFLAAALGVSGHYTWPRVNDGADWQQVRKADNWQDNGYVGDVTSPQIRCFQATPSPAPSVLNTTAGSTVTYWANPDVYHPGPVQFYMARVPDGEDINSWNGDGAVWFKVYEDHPTFGAQLTWPSTGKSSFAVPIPPCIKSGYYLLRAEQIGLHVAQSVGGAQFYISCAQLSVTGGGSTEPPNKVAFPGAYSATDPGILINIYYPVPTSYQNPGPAVFSC

SEQ ID NO: 10 - protein, 608 aa, Thielavia terrestris
MKGLFSAAALSLAVGQASAHYIFQQLSINGNQFPVYQYIRKNTNYNSPVTDLTSDDLRCNVGAQGAGTDTVTVKAGDQFTFTLDTPVYHQGPISIYMSKAPGAASDYDGSGGWFKIKDWGPTFNADGTATWDMAGSYTYNIPTCIPDGDYLLRIQSLAIHNPWPAGIPQFYISCAQITVTGGGNGNPGPTALIPGAFKDTDPGYTVNIYTNFHNYTVPGPEVFSCNGGGSNPPPPVSSSTPATTTLVTSTRTTSSTSSASTPASTGGCTVAKWGQCGGNGYTGCTTCAAGSTCSKQNDYYSQCLMKGLFSAAALSLAVGQASAHYIFQQLSINGNQFPVYQYIRKNTNYNSPVTDLTSDDLRCNVGAQGAGTDTVTVKAGDQFTFTLDTPVYHQGPISIYMSKAPGAASDYDGSGGWFKIKDWGPTFNADGTATWDMAGSYTYNIPTCIPDGDYLLRIQSLAIHNPWPAGIPQFYISCAQITVTGGGNGNPGPTALIPGAFKDTDPGYTVNIYTNFHNYTVPGPEVFSCNGGGSNPPPPVSSSTPATTTLVTSTRTTSSTSSASTPASTGGCTVAKWGQCGGNGYTGCTTCAAGSTCSKQNDYYSQCL

SEQ ID NO: 11 - protein, 250 aa, Thermoascus aurantiacus
MSFSKIIATAGVLASASLVAGHGFVQNIVIDGKKYYGGYLVNQYPYMSNPPEVIAWSTTATDLGFVDGTGYQTPDIICHRGAKPGALTAPVSPGGTVELQWTPWPDSHHGPVINYLAPCNGDCSTVDKTQLEFFKIAESGLINDDNPPGIWASDNLIAANNSWTVTIPTTIAPGNYVLRHEIIALHSAQNQDGAQNYPQCINLQVTGGGSDNPAGTLGTALYHDTDPGILINIYQKLSSYIIPGPPLYTG

SEQ ID NO: 12 - protein, 249 aa, Trichoderma reesei
MKSCAILAALGCLAGSVLGHGQVQNFTINGQYNQGFILDYYYQKQNTGHFPNVAGWYAEDLDLGFISPDQYTTPDIVCHKNAAPGAISATAAAGSNIVFQWGPGVWPHPYGPIVTYVVECSGSCTTVNKNNLRWVKIQEAGINYNTQVWAQQDLINQGNKWTVKIPSSLRPGNYVFRHELLAAHGASSANGMQNYPQCVNIAVTGSGTKALPAGTPATQLYKPTDPGILFNPYTTITSYTIPGPALWQG

SEQ ID NO: 13 - protein, 1097 aa, Aspergillus oryzae
MRSSPLLRSAVVAALPVLALAADGRSTRYWDCCKPSCGWAKKAPVNQPVFSCNANFQRITDFDAKSGCEPGGVAYSCADQTPWAVNDDFALGFAATSIAGSNEAGWCCACYELTFTSGPVAGKKMVVQSTSTGGDLGSNHFDLNIPGGGVGIFDGCTPQFGGLPGQRYGGISSRNECDRFPDALKPGCYWRFDWFKNADNPSFSFRQVQCPAELVARTGCRRNDDGNFPAVQIPMRSSPLLRSAVVAALPVLALAKDDLAYSPPFYPSPWADGQGEWAEVYKRAVDIVSQMTLTEKVNLTTGTGWQLERCVGQTGSVPRLNIPSLCLQDSPLGIRFSDYNSAFPAGVNVAATWDKTLAYLRGQAMGEEFSDKGIDVQLGPAAGPLGAHPDGGRNWESFSPDPALTGVLFAETIKGIQDAGVIATAKHYIMNEQEHFRQQPEAAGYGFNVSDSLSSNVDDKTMHELYLWPFADAVRAGVGAVMCSYNQINNSYGCENSETLNKLLKAELGFQGFVMSDWTAQHSGVGAALAGLDMSMPGDVTFDSGTSFWGANLTVGVLNGTIPQWRVDDMAVRIMAAYYKVGRDTKYTPPNFSSWTRDEYGFAHNHVSEGAYERVNEFVDVQRDHADLIRRIGAQSTVLLKNKGALPLSRKEKLVALLGEDAGSNSWGANGCDDRGCDNGTLAMAWGSGTANFPYLVTPEQAIQNEVLQGRGNVFAVTDSWALDKIAAAARQASVSLVFVNSDSGEGYLSVDGNEGDRNNITLWKNGDNVVKTAANNCNNTVVIIHSVGPVLIDEWYDHPNVTGILWAGLPGQESGNSIADVLYGRVNPGAKSPFTWGKTRESYGSPLVKDANNGNGAPQSDFTQGVFIDYRHFDKFNETPIYEFGYGLSYTTFELSDLHVQPLNASRYTPTSGMTEAAKNFGEIGDASEYVYPEGLERIHEFIYPWINSTDLKASSDDSNYGWEDSKYIPEGATDGSAQPRLPASGGAGGNPGLYEDLFRVSVKVKNTGNVAGDEVPQLYVSLGGPNEPKVVLRKFERIHLAPSQEAVWTTTLTRRDLANWDVSAQDWTVTPYPKTIYVGNSSRKLPLQASLPKAQ

SEQ ID NO: 14 - protein, 1097 aa, Aspergillus oryzae
MRSSPLLRSAVVAALPVLALAADGRSTRYWDCCKPSCGWAKKAPVNQPVFSCNANFQRITDFDAKSGCEPGGVAYSCADQTPWAVNDDFALGFAATSIAGSNEAGWCCACYELTFTSGPVAGKKMVVQSTSTGGDLGSNHFDLNIPGGGVGIFDGCTPQFGGLPGQRYGGISSRNECDRFPDALKPGCYWRFDWFKNADNPSFSFRQVQCPAELVARTGCRRNDDGNFPAVQIPMRSSPLLRSAVVAALPVLALAKDDLAYSPPFYPSPWADGQGEWAEVYKRAVDIVSQMTLTEKVNLTTGTGWQLERCVGQTGSVPRLNIPSLCLQDSPLGIRFSDYNSAFPAGVNVAATWDKTLAYLRGQAMGEEFSDKGIDVQLGPAAGPLGAHPDGGRNWEGFSPDPALTGVLFAETIKGIQDAGVIATAKHYIMNEQEHFRQQPEAAGYGFNVSDSLSSNVDDKTMHELYLWPFADAVRAGVGAVMCSYNQINNSYGCENSETLNKLLKAELGFQGFVMSDWTAHHSGVGAALAGLDMSMPGDVTFDSGTSFWGANLTVGVLNGTIPQWRVDDMAVRIMAAYYKVGRDTKYTPPNFSSWTRDEYGFAHNHVSEGAYERVNEFVDVQRDHADLIRRIGAQSTVLLKNKGALPLSRKEKLVALLGEDAGSNSWGANGCDDRGCDNGTLAMAWGSGTANFPYLVTPEQAIQNEVLQGRGNVFAVTDSWALDKIAAAARQASVSLVFVNSDSGEGYLSVDGNEGDRNNITLWKNGDNVVKTAANNCNNTVVIIHSVGPVLIDEWYDHPNVTGILWAGLPGQESGNSIADVLYGRVNPGAKSPFTWGKTRESYGSPLVKDANNGNGAPQSDFTQGVFIDYRHFDKFNETPIYEFGYGLSYTTFELSDLHVQPLNASRYTPTSGMTEAAKNFGEIGDASEYVYPEGLERIHEFIYPWINSTDLKASSDDSNYGWEDSKYIPEGATDGSAQPRLPASGGAGGNPGLYEDLFRVSVKVKNTGNVAGDEVPQLYVSLGGPNEPKVVLRKFERIHLAPSQEAVWTTTLTRRDLANWDVSAQDWTVTPYPKTIYVGNSSRKLPLQASLPKAQ

SEQ ID NO: 15 - protein, 532 aa, Aspergillus fumigatus
MLASTFSYRMYKTALILAALLGSGQAQQVGTSQAEVHPSMTWQSCTAGGSCTTNNGKVVIDANWRWVHKVGDYTNCYTGNTWDTTICPDDATCASNCALEGANYESTYGVTASGNSLRLNFVTTSQQKNIGSRLYMMKDDSTYEMFKLLNQEFTFDVDVSNLPCGLNGALYFVAMDADGGMSKYPTNKAGAKYGTGYCDSQCPRDLKFINGQANVEGWQPSSNDANAGTGNHGSCCAEMDIWEANSISTAFTPHPCDTPGQVMCTGDACGGTYSSDRYGGTCDPDGCDFNSFRQGNKTFYGPGMTVDTKSKFTVVTQFITDDGTSSGTLKEIKRFYVQNGKVIPNSESTWTGVSGNSITTEYCTAQKSLFQDQNVFEKHGGLEGMGAALAQGMVLVMSLWDDHSANMLWLDSNYPTTASSTTPGVARGTCDISSGVPADVEANHPDAYVVYSNIKVGPIGSTFNSGGSNPGGGTTTTTTTQPTTTTTTAGNPGGTGVAQHYGQCGGIGWTGPTTCASPYTCQKLNDYYSQCL

SEQ ID NO: 16 - protein, 454 aa, Aspergillus fumigatus
MKHLASSIALTLLLPAVQAQQTVWGQCGGQGWSGPTSCVAGAACSTLNPYYAQCIPGATATSTTLTTTTAATTTSQTTTKPTTTGPTTSAPTVTASGNPFSGYQLYANPYYSSEVHTLAMPSLPSSLQPKASAVAEVPSFVWLDVAAKVPTMGTYLADIQAKNKAGANPPIAGIFVVYDLPDRDCAALASNGEYSIANNGVANYKAYIDAIRAQLVKYSDVHTILVIEPDSLANLVTNLNVAKCANAQSAYLECVDYALKQLNLPNVAMYLDAGHAGWLGWPANLGPAATLFAKVYTDAGSPAAVRGLATNVANYNAWSLSTCPSYTQGDPNCDEKKYINAMAPLLKEAGFDAHFIMDTSRNGVQPTKQNAWGDWCNVIGTGFGVRPSTNTGDPLQDAFVWIKPGGESDGTSNSTSPRYDAHCGYSDALQPAPEAGTWFQAYFEQLLTNANPSF

SEQ ID NO: 17 - protein, 863 aa, Aspergillus fumigatus
MRFGWLEVAALTAASVANAQELAFSPPFYPSPWADGQGEWADAHRRAVEIVSQMTLAEKVNLTTGTGWEMDRCVGQTGSVPRLGINWGLCGQDSPLGIRFSDLNSAFPAGTNVAATWDKTLAYLRGKAMGEEFNDKGVDILLGPAAGPLGKYPDGGRIWEGFSPDPVLTGVLFAETIKGIQDAGVIATAKHYILNEQEHFRQVGEAQGYGYNITETISSNVDDKTMHELYLWPFADAVRAGVGAVMCSYNQINNSYGCQNSQTLNKLLKAELGFQGFVMSDWSAHHSGVGAALAGLDMSMPGDISFDDGLSFWGTNLTVSVLNGTVPAWRVDDMAVRIMTAYYKVGRDRLRIPPNFSSWTRDEYGWEHSAVSEGAWTKVNDFVNVQRSHSQIIREIGAASTVLLKNTGALPLTGKEVKVGVLGEDAGSNPWGANGCPDRGCDNGTLAMAWGSGTANFPYLVTPEQAIQREVISNGGNVFAVTDNGALSQMADVASQSSVSLVFVNADSGEGFISVDGNEGDRKNLTLWKNGEAVIDTVVSHCNNTIVVIHSVGPVLIDRWYDNPNVTAIIWAGLPGQESGNSLVDVLYGRVNPSAKTPFTWGKTRESYGAPLLTEPNNGNGAPQDDFNEGVFIDYRHFDKRNETPIYEFGHGLSYTTFGYSHLRVQALNSSSSAYVPTSGETKPAPTYGEIGSAADYLYPEGLKRITKFIYPWLNSTDLEDSSDDPNYGWEDSEYIPEGARDGSPQPLLKAGGAPGGNPTLYQDLVRVSATITNTGNVAGYEVPQLYVSLGGPNEPRVVLRKFDRIFLAPGEQKVWTTTLNRRDLANWDVEAQDWVITKYPKKVHVGSSSRKLPLRAPLPRVY

SEQ ID NO: 18 - protein, 253 aa, Penicillium sp.
MLSSTTRTLAFTGLAGLLSAPLVKAHGFVQGIVIGDQFYSGYIVNSFPYESNPPPVIGWATTATDLGFVDGTGYQGPDIICHRNATPAPLTAPVAAGGTVELQWTPWPDSHHGPVITYLAPCNGNCSTVDKTTLEFFKIDQQGLIDDTSPPGTWASDNLIANNNSWTVTIPNSVAPGNYVLRHEIIALHSANNKDGAQNYPQCINIEVTGGGSDAPEGTLGEDLYHDTDPGILVDIYEPIATYTIPGPPEPTF

SEQ ID NO: 19 - protein, 364 aa, Aspergillus fumigatus
MRFSLAATALLAGLATAAPSSNKNNVNLDKLARRNGMLWFGTAADIPGTSETTDKPYLSILRKQFGEMTPANALKVSQSDFMYTEPEQNVFNFTQGDYFMDLADHYGHAVRCHNLVWASQVSDWVTSRNWTATELKEVMKNHIFKTVQHFGKRCYAWDVVNEAINGDGTFSSSVWYDTIGEEYFYLAFQYAQEALAQIHANQVKLYYNDYGIENPGPKADAVLKLVAELRKRGIRIDGVGLESHFIVGETPSLADQLATKKAYIEAGLEVAITELDVRFSQAPFYTAEAQKQQAADYYASVASCKHAGPRCVGVVVWDFDDAYSWIPGTFEGQGGACLYNETLEVKPAFYAAAEALENKPCTVC

SEQ ID NO: 20 - protein, 323 aa, Aspergillus fumigatus
MVVLSKLVSSILFVSLVSAGVIDERQAAGINQAFTSHGKKYFGTASDQALLQKSQNEAIVRKDFGQLTPENSMKWDATEASQGRFNFAGADFLVNYAKQNGKKVRGHTLWHSQLPSWVSAISDKNTLTSVLKNHITTVMTRYKGQIYAWDVVNEIFNEDGSLRDSVFSRVLGEDFVRIAFETARSVDPSAKLYINDYKLDSASYGKTQGMVRYVKKWLAAGIPIDGIGQTHLGAGASSSVKGALTALASSGVSEVAITELDIAGASSQDYVNVVKACLDVPKCVGITVWGVSDRDSWRSGSSPLLFDSNYQPKAAYNAIIAAL

SEQ ID NO: 21 - protein, 397 aa, Aspergillus fumigatus
MVHLSSLAAALAALPLVYGAGLNTAAKAKGLKYFGSATDNPELTDSAYVAQLSNTDDFGQITPGNSMKWDATEPSQNSFSFANGDAVVNLANKNGQLMRCHTLVWHSQLPNWVSSGSWTNATLLAAMKNHITNVVTHYKGKCYAWDVVNEALNEDGTFRNSVFYQIIGPAYIPIAFATAAAADPDVKLYYNDYNIEYSGAKATAAQNIVKMIKAYGAKIDGVGLQAHFIVGSTPSQSDLTTVLKGYTALGVEVAYTELDIRMQLPSTAAKLAQQSTDFQGVAAACVSTTGCVGVTIWDWTDKYSWVPSVFQGYGAPLPWDENYVKKPAYDGLMAGLGASGSGTTTTTTTTSTTTGGTDPTGVAQKWGQCGGIGWTGPTTCVSGTTCQKLNDWYSQCL

SEQ ID NO: 22 - protein, 792 aa, Aspergillus fumigatus
MAVAKSIAAVLVALLPGALAQANTSYVDYNVEANPDLTPQSVATIDLSFPDCENGPLSKTLVCDTSARPHDRAAALVSMFTFEELVNNTGNTSPGVPRLGLPPYQVWSEALHGLDRANFTNEGEYSWATSFPMPILTMSALNRTLINQIATIIATQGRAFNNVGRYGLDVYAPNINAFRSAMWGRGQETPGEDAYCLASAYAYEYITGIQGGVDPEHLKLVATAKHYAGYDLENWDGHSRLGNDMNITQQELSEYYTPQFLVAARDAKVHSVMCSYNAVNGVPSCANSFFLQTLLRDTFGFVEDGYVSSDCDSAYNVWNPHEFAANITGAAADSIRAGTDIDCGTTYQYYFGEAFDEQEVTRAEIERGVIRLYSNLVRLGYFDGNGSVYRDLTWNDVVTTDAWNISYEAAVEGIVLLKNDGTLPLAKSVRSVALIGPWMNVTTQLQGNYFGPAPYLISPLNAFQNSDFDVNYAFGTNISSHSTDGFSEALSAAKKSDVIIFAGGIDNTLEAEAMDRMNITWPGNQLQLIDQLSQLGKPLIVLQMGGGQVDSSSLKSNKNVNSLIWGGYPGQSGGQALLDIITGKRAPAGRLVVTQYPAEYATQFPATDMSLRPHGNNPGQTYMWYTGTPVYEFGHGLFYTTFHASLPGTGKDKTSFNIQDLLTQPHPGFANVEQMPLLNFTVTITNTGKVASDYTAMLFANTTAGPAPYPNKWLVGFDRLASLEPHRSQTMTIPVTIDSVARTDEAGNRVLYPGKYELALNNERSVVLQFVLTGREAVIFKWPVEQQQISSA

SEQ ID NO: 23 - protein, 398 aa, Trichophaea saccata
MRTFSSLLGVALLLGAANAQVAVWGQCGGIGYSGSTTCAAGTTCVKLNDYYSQCQPGGTTLTTTTKPATTTTTTTATSPSSSPGLNALAQKSGRYFGSATDNPELSDAAYIAILSNKNEFGIITPGNSMKWDATEPSRGSFSFTGGQQIVDFAQGNGQAIRGHTLVWYSQLPSWVTSGNFDKATLTSIMQNHITTLVSHWKGQLAYWDVVNEAFNDDGTFRQNVFYTTIGEDYIQLAFEAARAADPTAKLCINDYNIEGTGAKSTAMYNLVSKLKSAGVPIDCIGVQGHLIVGEVPTTIQANLAQFASLGVDVAITELDIRMTLPSTTALLQQQAKDYVSVVTACMNVPRCIGITIWDYTDKYSWVPQTFSGQGDACPWDANLQKKPAYSAIASALAA

SEQ ID NO: 24 - protein, 796 aa, Talaromyces emersonii
MMTRTAILTALAALLPTATWAQDNQTYANYSSQSQPDLFPRTVATIDLSFPDCENGPLSTNLVCNTSADPWARAEALVSLFTLEELINNTQNTAPGVPRLGLPQYQVWNEALHGLDRANFSDSGEYSWATSFPMPILSMASFNRTLINQIASIIATQARAFNNAGRYGLDSYAPNINGFRSPLWGRGQETPGEDAFFLSSAYAYEYITGLQGGVDPEHVKIVATAKHFAGYDLENWGNVSRLGFNAIITQQDLSEYYTPQFLASARYAKTRSLMCSYNAVNGVPSCSNSFFLQTLLRERFNFVDDGYVSSDCDAVYNVFNPHGYALNQSGAAADSLLAGTDIDCGQTMPWHLNESFYEGYVSRGDIEKSLTRLYANLVRLGYFDGNNSVYRNLNWNDVVTTDAWNISYEAAVEGITLLKNDRTLPLSKKVRSIALIGPWANATVQMQGNYYGTPPYLISPLEAAKASGFTVNYAFGTNISTDSTQWFAEAISAAKKSDVIIYAGGIDNTIEAEGQDRTDLKWPGNQLDLIEQLSKVGKPLVVLQMGGGQVDSSSLKANKNVNALVWGGYPGQSGGAALFDILTGKRAPAGRLVSTQYPAEYATQFPANDMNLRPNGSNPGQTYIWYTGTPVYEFGHGLFYTEFQESAAAGTNKTSTFDILDLVSTPHPGYEYIELVPFLNVTVDVKNVGHIPSPYTGLLFANTTAGPKPYPNKWLVGFDRLPTIQPGETAQVTFPVPLGAIARADENGNKVIFPGDYELALNNERSVVVSFSLTGNAATLENWPVWEQAVPGVLQQ

SEQ ID NO: 25 - protein, 532 aa, Talaromyces leycettanus
MASLFSFKMYKAALVLSSLLAATQAQQAGTLTTETHPSLTWQQCSAGGSCTTQNGKVVIDANWRWVHSTSGSNNCYTGNTWDATLCPDDVTCAANCALDGADYSGTYGVTTSGNSLRLNFVTQASQKNVGSRLYLMENDTTYQIFKLLNQEFTFDVDVSNLPCGLNGALYLVAMDADGGMAKYPTNKAGAKYGTGYCDSQCPRDLKFINGEANVEGWQPSSNDPNSGIGNHGSCCAEMDIWEANSISNAVTPHPCDTPGQVMCTGNNCGGTYSTTRYAGTCDPDGCDFNPYRMGNHSFYGPKQIVDTSSKFTVVTQFLTDDGTSTGTLSEIRRFYVQNGQVIPNSVSTISGVSGNSITTEFCTAQKQAFGDTDDFSKHGGLSGMSAALSQGMVLVMSLWDDHAANMLWLDSTYPTNATSSTPGAARGTCDISSGVPADVESNDPNAYVVYSNIKVGPIGSTFSSSGSGSSSSSSTTTTTTASPTTTTSSASSTGTGVAQHWGQCGGQGWTGPTTCVSPYTCQELNPYYYQCL

SEQ ID NO: 26 - protein, 464 aa, Talaromyces leycettanus
MRSLLALAPTLLAPVVQAQQTMWGQCGGQGWTGPTICVAGATCSTQNPWYAQCTPAPTAPTTLQTTTTTSSKSSTTTSSKSSTTTGGSGGGTTTSTSATITAAPSGNPYSGYQLYVNQEYSSEVYASAIPSLTGTLVAKASAAAEVPSFLWLDTASKVPLMGTYLQDIQAKNAAGANPPYAGQFVVYDLPDRDCAALASNGEYSIANNGVANYKAYIDSIRALLVQYSNVHVILVIEPDSLANLVTNLNVQKCANAQSAYLECINYALTQLNLKNVAMYIDAGHAGWLGWPANLSPAAQLFASVYQNASSPAAVRGLATNVANYNAWSIATCPSYTQGDPNCDEQKYINALAPLLQQQGWSSVHFITDTGRNGVQPTKQNAWGDWCNVIGTGFGVRPTTNTGDPLEDAFVWVKPGGESDGTSNSTSPRYDAHCGYSDALQPAPEAGTWFEAYFEQLLTNANPSF

SEQ ID NO: 27 - protein, 405 aa, Talaromyces leycettanus
MVHLSSLALALAAGSQLAQAAGLNTAAKAIGKLYFGTATDNPELSDSTYMQETDNTDDFGQLTPANSMKWDATEPSQNTFTFTNGDQIANLAKSNGQMLRCHNLVWYNQLPSWVTSGSWTNATLLAAMKNHITNVVTHYKGQCYAWDVVNEALNDDGTYRSNVFYQYIGEAYIPIAFATAAAADPNAKLYYNDYNIEYPGAKATAAQNIVKMVKAYGAKIDGVGLQSHFIVGSTPSQSSQQSNMAAFTALGVEVAITELDIRMTLPSTSALLAQQSTDYQSTVSACVNTPKCIGITLWDWTDKYSWVPNTFSGQGDACPWDSNYQKKPAYYGILTALGGSASTSTTTTLVTSTRTSTTTSTSATSTSTGVAQHWGQCGGIGWTGPTTCASPYTCQELNPYYYQCL

SEQ ID NO: 28 - protein, 398 aa, Trichophaea saccata; identical to SEQ ID NO: 23

SEQ ID NO: 29 - protein, 459 aa, Trichoderma reesei
MAPSVTLPLTTAILAIARLVAAQQPGTSTPEVHPKLTTYKCTKSGGCVAQDTSVVLDWNYRWMHDANYNSCTVNGGVNTTLCPDEATCGKNCFIEGVDYAASGVTTSGSSLTMNQYMPSSSGGYSSVSPRLYLLDSDGEYVMLKLNGQELSFDVDLSALPCGENGSLYLSQMDENGGANQYNTAGANYGSGYCDAQCPVQTWRNGTLNTSHQGFCCNEMDILEGNSRANALTPHSCTATACDSAGCGFNPYGSGYKSYYGPGDTVDTSKTFTIITQFNTDNGSPSGNLVSITRKYQQNGVDIPSAQPGGDTISSCPSASAYGGLATMGKALSSGMVLVFSIWNDNSQYMNWLDSGNAGPCSSTEGNPSNILANNPNTHVVFSNIRWGDIGSTTNSTAPPPPPASSTTFSTTRRSSTTSSSPSCTQTHWGQCGGIGYSGCKTCTSGTTCQYSNDYYSQCL

SEQ ID NO: 30 - protein, 418 aa, Trichoderma reesei
MNKSVAPLLLAASILYGGAAAQQTVWGQCGGIGWSGPTNCAPGSACSTLNPYYAQCIPGATTITTSTRPPSGPTTTTRATSTSSSTPPTSSGVRFAGVNIAGFDFGCTTDGTCVTSKVYPPLKNFTGSNNYPDGIGQMQHFVNDDGMTIFRLPVGWQYLVNNNLGGNLDSTSISKYDQLVQGCLSLGAYCIVDIHNYARWNGGIIGQGGPTNAQFTSLWSQLASKYASQSRVWFGIMNEPHDVNINTWAATVQEVVTAIRNAGATSQFISLPGNDWQSAGAFISDGSAAALSQVTNPDGSTTNLIFDVHKYLDSDNSGTHAECTTNNIDGAFSPLATWLRQNNRQAILTETGGGNVQSCIQDMCQQIQYLNQNSDVYLGYVGWGAGSFDSTYVLTETPTGSGNSWTDTSLVSSCLARK

SEQ ID NO: 31 - protein, 335 aa, Thermoascus aurantiacus
MKLGSLVLALSAARLTLSAPLADRKQETKRAKVFQWFGSNESGAEFGSQNLPGVEGKDYIWPDPNTIDTLISKGMNIFRVPFMMERLVPNSMTGSPDPNYLADLIATVNAITQKGAYAVVDPHNYGRYYNSIISSPSDFQTFWKTVASQFASNPLVIFDTNNEYHDMDQTLVLNLNQAAIDGIRSAGATSQYIFVEGNSWTGAWTWTNVNDNMKSLTDPSDKIIYEMHQYLDSDGSGTSATCVSSTIGQERITSATQWLRANGKKGIIGEFAGGANDVCETAITGMLDYMAQNTDVWTGAIWWAAGPWWGDYIFSMEPDNGIAYQQILPILTPYL

SEQ ID NO: 32 - protein, 269 aa, Bacillus clausii
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 33 - protein, 274 aa, Bacillus licheniformis
AQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGTYSGIVSGIEWATTNGMDVINMSLGGPSGSTAMKQAVDNAYARGVVVVAAAGNSGSSGNTNTIGYPAKYDSVIAVGAVDSNSNRASFSSVGAELEVMAPGAGVYSTYPTSTYATLNGTSMASPHVAGAAALILSKHPNLSASQVRNRLSSTATYLGSSFYYGKGLINVEAAAQ

SEQ ID NO: 34 - protein, 521 aa, Bacillus amyloliquefaciens
MKKPLGKIVASTALLISVAFSSSIASAAENPQLKEDLTNFVPKHSLVQSELPSVSDKAIKQYLKQNGKVFKGNPSERLKLIDQTTDDLGYKHFRYVPVVNGVPVKDSQVIIHVDKSNNVYAINGELNNDVSAKTANSKKLSANQALDHAYKAIGKSPEAVSNGTVANKNKAELKAAATKDGKYRLAYDVTIRYIEPEPANWEVTVDAETGKILKKQNKVEHAATTGTGTTLKGKTVSLNISSESGKYVLRDLSKPTGTQIITYDLQNREYNLPGTLVSSTTNQFTTSSQRAAVDAHYNLGKVYDYFYQKFNRNSYDNKGGKIVSSVHYGSRYNNAAWIGDQMIYGDGDGSFFSPLSGSMDVTAHEMTHGVTQETANLNYENQPGALNESFSDVFGYFNDTEDWDIGEDITVSQPALRSLSNPTKYGQPDNFKNYKNLPNTDAGDYGGVHTNSGIPNKAAYNTITKIGVNKAEQIYYRALTVYLTPSSTFKDAKAALIQSARDLYGSQDAASVEAAWNAVGL

SEQ ID NO: 35 - protein, 274 aa, Thermomyces lanuginosus
EVSQDLFNQFNLFAQYSAAAYCGKNNDAPAGTNITCTGNACPEVEKADATFLYSFEDSGVGDVTGFLALDNTNKLIVLSFRGSRSIENWIANLNFWLKKINDICSGCRGHDGFTSSWRSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHSSPEYWIKSGTLVPVTRNDIVKIEGIDATGGNNQPNIPDIPAHLWYFQATDACNAGGFS

SEQ ID NO: 36 - protein, 564 aa, Meripilus giganteus
MVATSLLVASLFTLALGTPTGRNLKLHEAREDLPAGFSLRGAASPDTTLKLRIALVQNNFAELEDKLYDVSTPSSANYGNHLSKEEVEQYIAPAPESVKAVNAWLTENGLDAHTISPAGDWLAFEVPVSKANELFDADFSVFTHDESGLEAIRTLAYSIPAELQGHLDLVHPTVTFPNPNAHLPVVRSTQPIRNLTGRAIPASCASTITPACLQAIYGIPTTKATQSSNKLAVSGFIDQFANKADLKSFLAQFRKDISSSTTFSLQTLDGGENDQSPSEAGIEANLDIQYTVGLATGVPTTFISVGDDFQDGNLEGFLDIINFLLGESNPPQVLTTSYGQNENTISAKLANQLCNAYAQLGARGTSILFASGDGGVSGSQSAHCSNFVPTFPSGCPFMTSVGATQGVSPETAAAFSSGGFSNVFGIPSYQASAVSGYLSALGSTNSGKFNRSGRGFPDVSTQGVDFQIVSGGQTIGVDGTSCASPTFASVISLVNDRLIAAGKSPLGFLNPFLYSSAGKAALNDVTSGSNPGCSTNGFPAKAGWDPVTGLGTPNFAKLLTAVGL